(19) United States
(12) Patent Application Publication
(10) Pub. No.: US 2024/0319597 A1
(43) Pub. Date: Sep. 26, 2024

(54) RADIATION-SENSITIVE RESIN COMPOSITION, PATTERN FORMATION METHOD, METHOD FOR MANUFACTURING SUBSTRATE, AND COMPOUND

(71) Applicant: JSR CORPORATION, Tokyo (JP)

(72) Inventors: Ryuichi NEMOTO, Tokyo (JP); Michihiro MITA, Tokyo (JP); Masayuki MIYAKE, Tokyo (JP)

(73) Assignee: JSR CORPORATION, Tokyo (JP)

(21) Appl. No.: 18/678,331

(22) Filed: May 30, 2024

Related U.S. Application Data
(63) Continuation-in-part of application No. PCT/JP2022/040696, filed on Oct. 31, 2022.

(30) Foreign Application Priority Data
Dec. 1, 2021 (JP) 2021-195173

(51) Int. Cl. G03F 7/039; G03F 7/004; G03F 7/038; G03F 7/32

(52) U.S. Cl. CPC G03F 7/0397; G03F 7/0045; G03F 7/0382; G03F 7/322; G03F 7/325

(57) ABSTRACT

A radiation-sensitive resin composition includes: a compound A represented by formula (I); a resin B including a structural unit having an acid-dissociable group; a radiation-sensitive acid generator other than the compound A; and a solvent. R¹ is an (m+m′)-valent organic group and comprises a cyclopropane ring skeleton, a cyclobutane ring skeleton, or both; X¹ is a group represented by formula (1-1) or a group represented by formula (1-2); X² is a group represented by formula (2-1) or a group represented by formula (2-2); Y⁺ is a monovalent onium cation; m is an integer of 1 to 2, and m′ is an integer of 0 to 1. * represents a bond to another group.

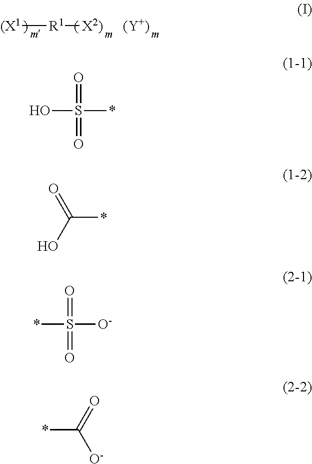

### CROSS-REFERENCE TO RELATED APPLICATIONS

The present application is a continuation-in-part application of International Patent Application No. PCT/JP2022/040696 filed Oct. 31, 2022, which claims priority to Japanese Patent Application No. 2021-195173 filed Dec. 1, 2021. The contents of these applications are incorporated herein by reference in their entirety.

### BACKGROUND OF THE DISCLOSURE

### Technical Field

The present disclosure relates to a radiation-sensitive resin composition, a pattern formation method, a method for manufacturing a substrate, and a compound.

### Background Art

A photolithography technology using a resist composition has been used for the formation of a fine circuit in a semiconductor device. As a representative procedure, for example, a resist pattern is formed on a substrate by generating an acid by irradiating a coating film of the resist composition with radiation through a mask pattern, and then reacting in the presence of the acid as a catalyst to generate a difference in the solubility of a resin into an alkaline or organic solvent-based developer between an exposed area and an unexposed area.

In the photolithography technology, pattern miniaturization is promoted by using short-wavelength radiation, such as ArF excimer laser or by combining such radiation with an immersion exposure method (liquid immersion lithography). As a next-generation technology, further short-wavelength radiation, such as an electron beam, an X-ray, and an extreme ultraviolet ray (EUV) is being utilized, and a resist material containing an acid generator with a benzene ring having enhanced radiation absorption efficiency is also being studied (JP-A-2014-2359).

### SUMMARY

According to an aspect of the present disclosure, a radiation-sensitive resin composition includes: a compound A represented by formula (I); a resin B including a structural unit having an acid-dissociable group; a radiation-sensitive acid generator other than the compound A; and a solvent.

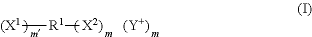

R¹ is an (m+m′)-valent organic group and comprises a cyclopropane ring skeleton, a cyclobutane ring skeleton, or both; X¹ is a group represented by formula (1-1) or a group represented by formula (1-2); X² is a group represented by formula (2-1) or a group represented by formula (2-2); Y⁺ is a monovalent onium cation; m is an integer of 1 to 2, and m′ is an integer of 0 to 1.

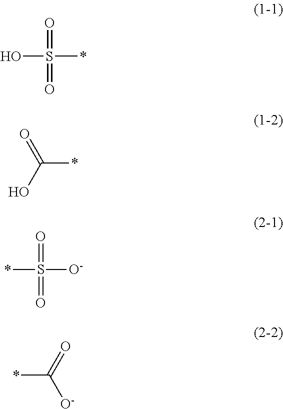

* represents a bond to another group.

According to another aspect of the present disclosure, a pattern formation method, includes: applying the above-described radiation-sensitive resin composition directly or indirectly to a substrate to form a resist film; exposing the resist film to light; and developing the exposed resist film to form a patterned resist film.

According to a further aspect of the present disclosure, a method for manufacturing a substrate includes forming a pattern on a substrate using the patterned resist film formed by the above-described pattern formation method as a mask.

According to a further aspect of the present disclosure, a compound is represented by formula (I):

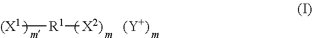

R¹ is an (m+m′)-valent organic group and comprises a cyclopropane ring skeleton, a cyclobutane ring skeleton, or both; X¹ is a group represented by formula (1-1) or a group represented by formula (1-2); X² is a group represented by formula (2-1) or a group represented by formula (2-2), Y⁺ is a monovalent onium cation, m is an integer of 1 to 2, and m′ is an integer of 0 to 1.

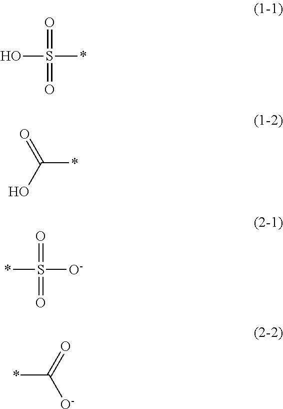

* represents a bond to another group.

### DESCRIPTION OF THE EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

Even in the above-described next generation technology, various resist performances equivalent to or higher than conventional performances are required in sensitivity and critical dimension uniformity (CDU) performance, which is an index of uniformity of a line width and a hole diameter, and the like.

The present disclosure relates, in one embodiment, to a radiation-sensitive resin composition comprising:
a compound A represented by the following formula (I),

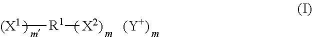

wherein
R¹ is an (m+m′)-valent organic group and contains a cyclopropane ring skeleton, a cyclobutane ring skeleton, or both,
X¹ is a group represented by the following formula (1-1) or a group represented by the following formula (1-2),
X² is a group represented by the following formula (2-1) or a group represented by the following formula (2-2),
Y⁺ is a monovalent onium cation,
m is an integer of 1 to 2, and
m′ is an integer of 0 to 1,

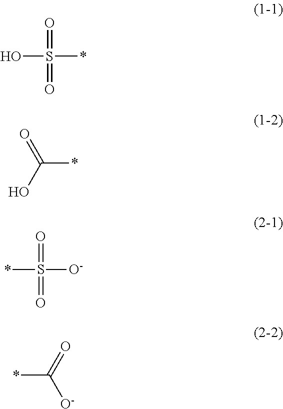

represents a bond to another group,
a resin B containing a structural unit having an acid-dissociable group;
a radiation-sensitive acid generator other than the compound A; and
a solvent.

The present disclosure relates, in another embodiment, to a pattern formation method, the method including the steps of:
a step (1) of applying the radiation-sensitive resin composition directly or indirectly to a substrate to form a resist film;
a step (2) of exposing the resist film to light; and
a step (3) of developing the exposed resist film.

In addition, the present disclosure relates, in another embodiment,
to a method for manufacturing a substrate, the method comprising a step (4) of forming a pattern on a substrate using a pattern formed by the above-described method as a mask.

In addition, the present disclosure relates, in another embodiment,
to a compound represented by the following formula (I),

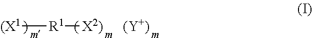

wherein
R¹ is an (m+m′)-valent organic group and contains a cyclopropane ring skeleton, a cyclobutane ring skeleton, or both,
X¹ is a group represented by the following formula (1-1) or a group represented by the following formula (1-2),
X² is a group represented by the following formula (2-1) or a group represented by the following formula (2-2),
Y⁺ is a monovalent onium cation,
m is an integer of 1 to 2, and
m′ is an integer of 0 to 1,

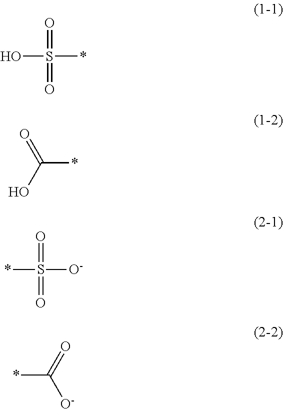

wherein * represents a bond to another group.

By the use of the radiation-sensitive resin composition of the present disclosure, a resist film satisfying sensitivity and CDU performance can be constructed.

In accordance with the pattern formation method of the present disclosure, by using the radiation-sensitive resin composition superior in sensitivity and CDU performance, a high-quality resist pattern can be efficiently formed.

In addition, by using the method for manufacturing a substrate of the present disclosure, a high-quality substrate can be efficiently formed.

By using the compound of the present disclosure, the radiation-sensitive resin composition can be obtained.

Hereinbelow, embodiments of the present invention will specifically be described, but the present invention is not limited to these embodiments.

### <Radiation-Sensitive Resin Composition>

The radiation-sensitive resin composition (hereinafter, also simply referred to as “composition”) according to the present embodiment includes a compound that is a prescribed onium salt (hereinafter, the compound is also referred to as “compound” or “compound A”), a resin containing a structural unit having an acid-dissociable group (hereinafter, the resin is also referred to as “resin B”), a radiation-sensitive acid generator other than the compound A, and a solvent. In addition, the composition contains another resin, as necessary. The composition may further contain other optional components as long as the effects of the present invention are not impaired. When the radiation-sensitive resin composition contains the prescribed compound, the radiation-sensitive resin composition can be provided with high levels of sensitivity and CDU performance.

In the present description, the organic group refers to a group containing at least one carbon atom.

### <Compound>

The compound (compound A) is represented by the formula (I).

Since the compound A has a small anion moiety with high transparency containing a cyclopropane ring skeleton, a cyclobutane ring skeleton, or both, it is presumed that the radiation-sensitive resin composition containing the compound A can construct a resist film satisfying sensitivity and CDU performance.

In the formula (I), R¹ is an (m+m′)-valent organic group and contains a cyclopropane ring skeleton, a cyclobutane ring skeleton, or both.

The cyclopropane ring skeleton may be any skeleton containing a cyclopropane ring structure.

The cyclobutane ring skeleton may be any skeleton containing a cyclobutane ring structure.

In the formula (I), X¹ is a group represented by the formula (1-1) or a group represented by the formula (1-2).

In the formula (I), X² is a group represented by the formula (2-1) or a group represented by the formula (2-2).

In the formula (I), m is an integer of 1 to 2. When m is 2, a plurality of X¹ is the same or different.

In the formula (I), m′ is an integer of 0 to 1.

In the formula (I), Y⁺ is a monovalent onium cation.

The Y⁺ is preferably a monovalent radiolytic onium cation.

Examples of the monovalent onium cation include radiation-sensitive onium cations containing such elements as S, I, O, N, P, Cl, Br, F, As, Se, Sn, Sb, Te, and Bi. Examples of the cation containing S (sulfur) as an element include a sulfonium cation and a tetrahydrothiophenium cation, and examples of the cation containing I (iodine) as an element include an iodonium cation.

Examples of the Y⁺ include a cation represented by the following formula (Q-1) (hereinafter, also referred to as “cation (Q-1)”), a cation represented by the following formula (Q-2) (hereinafter, also referred to as “cation (Q-2)”), and a cation represented by the following formula (Q-3) (hereinafter, also referred to as “cation (Q-3)”).

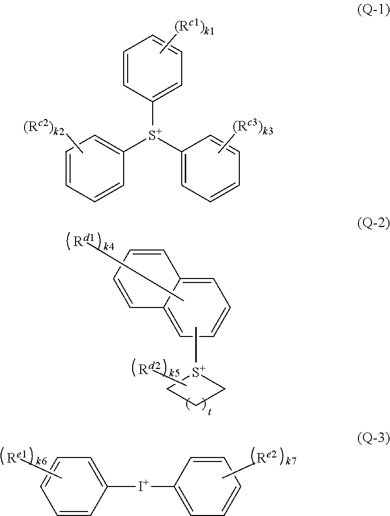

In the formula (Q-1), R^{c1}, R^{c2} and R^{c3} each independently represent a substituted or unsubstituted linear or branched alkyl group having 1 to 12 carbon atoms, a substituted or unsubstituted aromatic hydrocarbon group having 6 to 12 carbon atoms, —OSO₂—RP′ or —SO₂—RQ′, or a ring structure constituted by combining two or more of these groups with each other. RP′ and RQ′ are each independently a substituted or unsubstituted linear or branched alkyl group having 1 to 12 carbon atoms, a substituted or unsubstituted alicyclic hydrocarbon group having 5 to 25 carbon atoms, or a substituted or unsubstituted aromatic hydrocarbon group having 6 to 12 carbon atoms. k1, k2, and k3 each independently are an integer of 0 to 5. When there are pluralities of R^{c1}s to R^{c3}s, RP's and RQ's, the pluralities of R^{c1}s to R^{c3}s, RP's and RQ's each may be either identical or different.

In the above formula (Q-2), R^{d1} is a substituted or unsubstituted linear or branched alkyl group having 1 to 8 carbon atoms or a substituted or unsubstituted aromatic hydrocarbon group having 6 to 8 carbon atoms. k4 is an integer of 0 to 7. When there is a plurality of R^{d1}s, the plurality of R^{d1}s may be either identical or different, and the plurality of R^{d1}s may represent a ring structure constituted by combining the R^{d1}s with each other. R^{d2} is a substituted or unsubstituted linear or branched alkyl group having 1 to 7 carbon atoms or a substituted or unsubstituted aromatic hydrocarbon group having 6 or 7 carbon atoms. k5 is an integer of 0 to 6. When there is a plurality of R^{d2}s, the plurality of R^{d2}s may be either identical or different, and the plurality of R^{d2}s may represent a ring structure constituted by combining the R^{d2}s with each other. t is an integer of 0 to 3.

In the formula (Q-3), R^{e1} and R^{e2} each independently represent a substituted or unsubstituted linear or branched alkyl group having 1 to 12 carbon atoms, a substituted or unsubstituted aromatic hydrocarbon group having 6 to 12 carbon atoms, —OSO₂—R^{R} or —SO₂—R^{S}, or a ring structure constituted by combining two or more of these groups with each other. R^{R} and R^{S} are each independently a substituted or unsubstituted linear or branched alkyl group having 1 to 12 carbon atoms, a substituted or unsubstituted alicyclic hydrocarbon group having 5 to 25 carbon atoms, or a substituted or unsubstituted aromatic hydrocarbon group having 6 to 12 carbon atoms. k6 and k7 each independently are an integer of 0 to 5. When there are pluralities of R^{e1}s, R^{e2}s, R^{R}s, and R^{S}s, the pluralities of R^{e1}s, R^{e2}s, R^{R}s, and R^{S}s each may be either identical or different.

Examples of the unsubstituted linear alkyl groups represented by R^{c1} to R^{c3}, R^{d1}, R^{d2}, R^{e1} and R^{e2} include a methyl group, an ethyl group, a n-propyl group, and a n-butyl group.

Examples of the unsubstituted branched alkyl groups represented by R^{c1} to R^{c3}, R^{d1}, R^{d2}, R^{e1} and R^{e2} include an i-propyl group, an i-butyl group, a sec-butyl group, and a t-butyl group.

Examples of the unsubstituted aromatic hydrocarbon groups represented by R^{c1} to R^{c3}, R^{e1} and R^{e2} include aryl groups such as a phenyl group, a tolyl group, a xylyl group, a mesityl group, and a naphthyl group; and aralkyl groups such as a benzyl group and a phenethyl group.

Examples of the unsubstituted aromatic hydrocarbon groups represented by R^{d1} and R^{d2} include a phenyl group, a tolyl group, and a benzyl group.

Examples of the substituent that may substitute a hydrogen atom of the alkyl group or the aromatic hydrocarbon group include halogen atoms such as a fluorine atom, a chlorine atom, a bromine atom, and an iodine atom, a hydroxy group, a carboxy group, a cyano group, a nitro group, an alkoxy group, an alkoxycarbonyl group, an alkoxycarbonyloxy group, an acyl group, and an acyloxy group. Among them, halogen atoms are preferable, and a fluorine atom is more preferable.

As R^{c1} to R^{c3}, R^{d1}, R^{d2}, R^{e1} and R^{e2}, unsubstituted linear or branched alkyl groups, fluorinated alkyl groups, unsubstituted monovalent aromatic hydrocarbon groups, —OSO₂—R**, and —SO₂—R** are preferable, fluorinated alkyl groups and unsubstituted monovalent aromatic hydrocarbon groups are more preferable, and fluorinated alkyl groups are still more preferable. R** is an unsubstituted monovalent alicyclic hydrocarbon group or an unsubstituted monovalent aromatic hydrocarbon group.

As k1, k2, and k3 in the formula (Q-1), integers of 0 to 2 are preferable, 0 or 1 is more preferable, and 0 is still more preferable.

As k4 in the formula (Q-2), integers of 0 to 2 are preferable, 0 or 1 is more preferable, and 1 is still more preferable. As k5, integers of 0 to 2 are preferable, 0 or 1 is more preferable, and 0 is still more preferable.

As k6 and k7 in the formula (Q-3), integers of 0 to 2 are preferable, 0 or 1 is more preferable, and 0 is still more preferable.

Examples of the cation (Q-1) include cations represented by the following formulas (i-1) to (i-21).

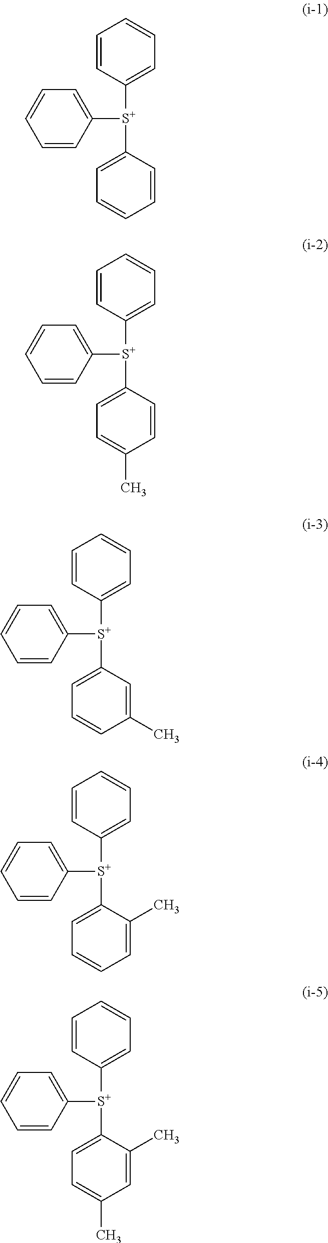 [SMILES: c1ccc([S+](c2ccccc2)c2ccccc2)cc1 ; Cc1ccc([S+](c2ccccc2)c2ccccc2)cc1 ; Cc1cccc([S+](c2ccccc2)c2ccccc2)c1 ; Cc1ccccc1[S+](c1ccccc1)c1ccccc1 ; Cc1ccc([S+](c2ccccc2)c2ccccc2)c(C)c1]

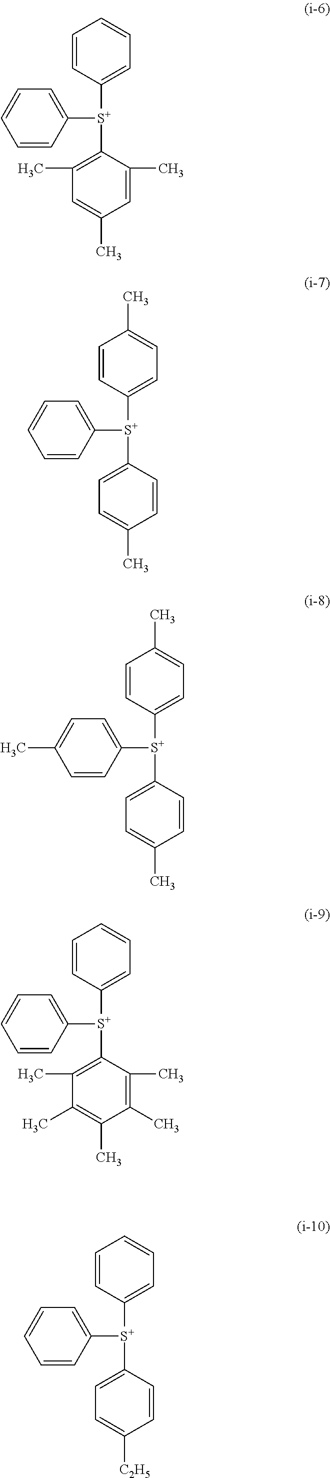

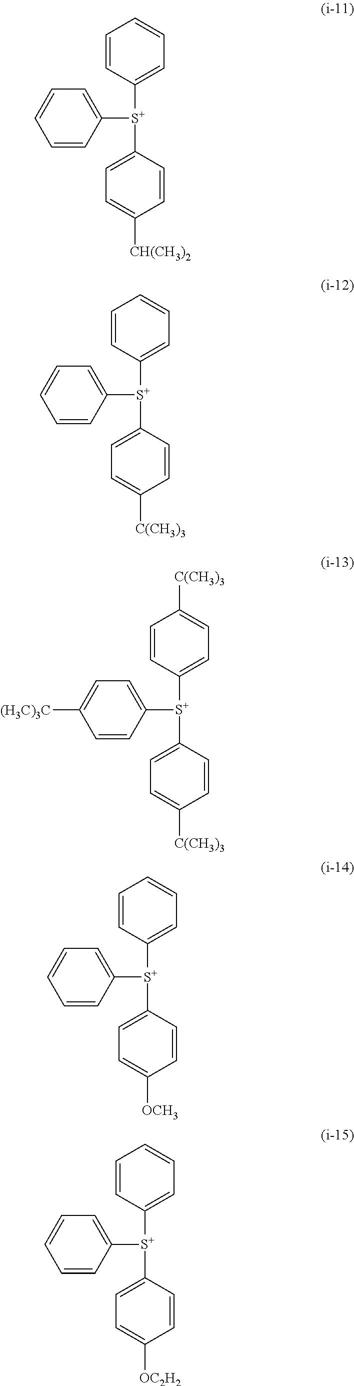

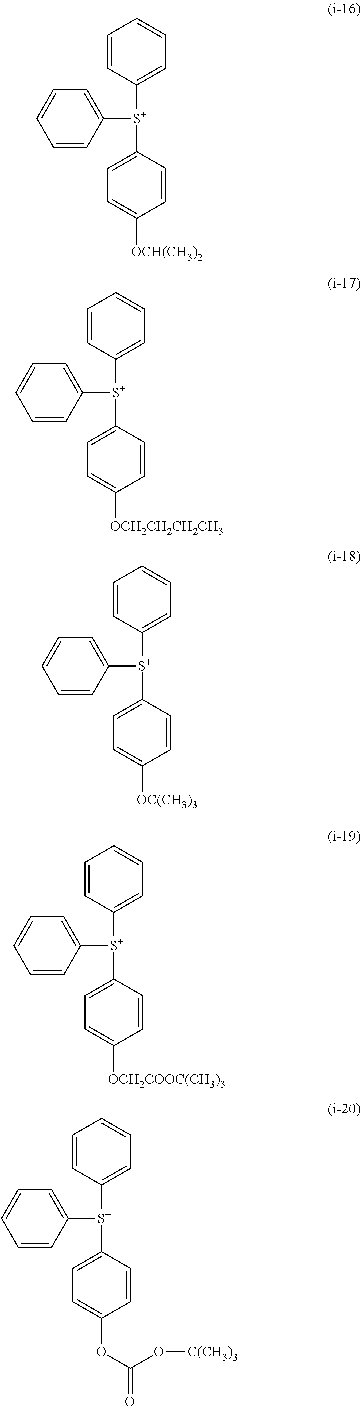

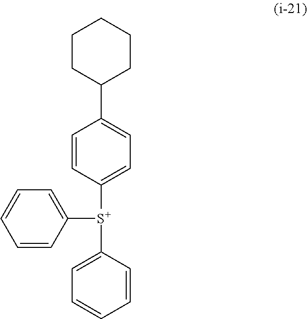 [SMILES: c1ccc([S+](c2ccccc2)c2ccc(C3CCCCC3)cc2)cc1]

Among them, the cation represented by the formula (i-1) and the cation represented by the formula (i-21) are preferable.

Examples of the cation (Q-2) include cations represented by the following formulas (i′-1) to (i′-4).

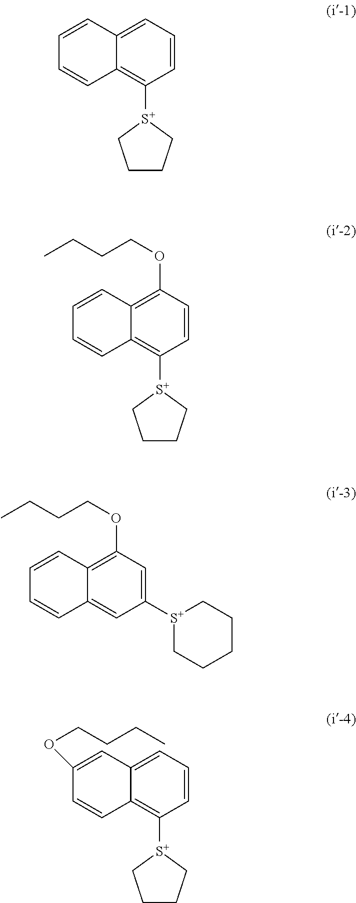 [SMILES: c1ccc2c([S+]3CCCC3)cccc2c1 ; CCCCOc1ccc([S+]2CCCC2)c2ccccc12 ; CCCCOc1cc([S+]2CCCCC2)cc2ccccc12 ; CCCCOc1ccc2c([S+]3CCCC3)cccc2c1]

Among them, for example, the cation represented by the formula (i′-2) is preferable.

Examples of the cation (Q-3) include cations represented by the following formulas (ii-1) to (ii-25).

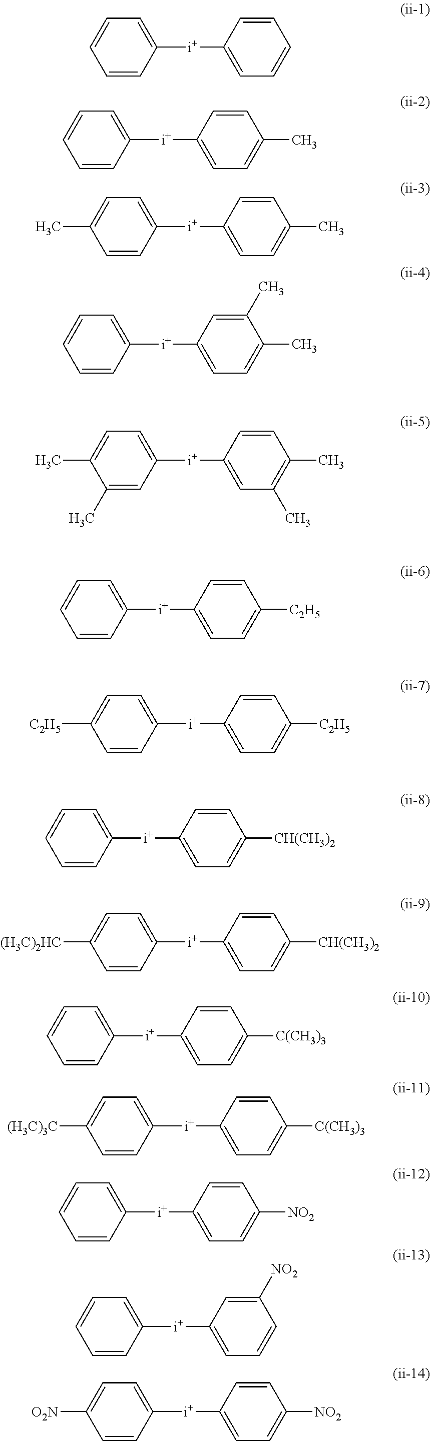

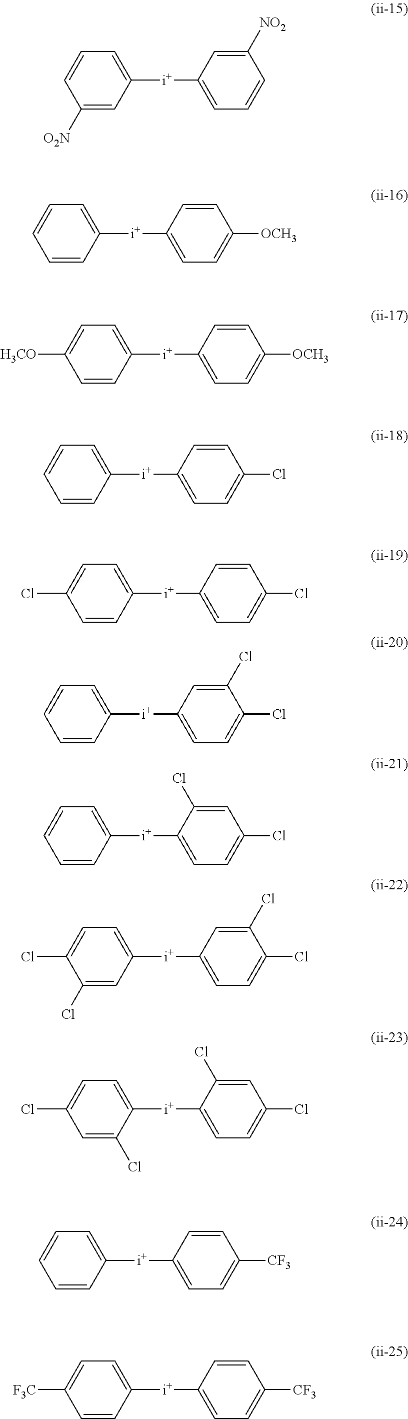

Among them, the cation represented by the formula (ii-11) is preferable.

In addition, the compound A is preferably, for example, a compound represented by the following formula (1) or (2).

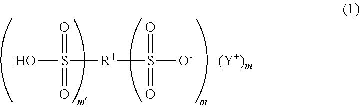

wherein R¹, Y⁺, m, and m′ are the same as those in the formula (I).

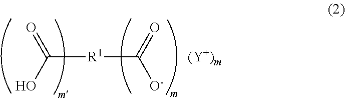

wherein R¹, Y⁺, m, and m′ are the same as those in the formula (I).

In addition, the compound A is preferably, for example, a compound represented by the following formula (3).

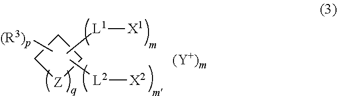

wherein
R³ is a monovalent organic group, a fluorine atom, or a hydroxy group,
L¹ and L² are each independently a single bond or a divalent organic group,
X¹, X², Y⁺, m, and m′ are the same as those in the formula (I),
Z is a divalent group represented by —C(R⁴)₂— or —CO—,
R⁴ is independently at each occurrence a hydrogen atom, a monovalent organic group, a fluorine atom, or a hydroxy group,
q is an integer of 0 to 1, and
p is an integer of 0 to (6−m−m′).

In the formula (3), R³ is a monovalent organic group, a fluorine atom, or a hydroxy group,

Examples of the monovalent organic group include a monovalent hydrocarbon group.

Examples of the monovalent hydrocarbon group include a chain hydrocarbon group, an alicyclic hydrocarbon group, and an aromatic hydrocarbon group.

Examples of the chain hydrocarbon group include:
alkyl groups such as a methyl group, an ethyl group, a n-propyl group, an i-propyl group, a n-butyl group, an i-butyl group, a sec-butyl group, and a t-butyl group;
alkenyl groups such as an ethenyl group, a propenyl group, and a butenyl group; and
alkynyl groups such as an ethynyl group, a propynyl group, and a butynyl group.

Examples of the alicyclic hydrocarbon group include:
monocyclic cycloalkyl groups such as a cyclopropyl group, a cyclobutyl group, a cyclopentyl group, a cyclohexyl group, and a cyclooctyl group;
monocyclic cycloalkenyl groups such as a cyclobutenyl group, a cyclopentenyl group, and a cyclohexenyl group;
polycyclic cycloalkyl groups such as a norbornyl group, an adamantyl group, a tricyclodecyl group, and a tetracyclododecyl group; and
polycyclic cycloalkenyl groups such as a norbornenyl group, a tricyclodecenyl group, and a tetracyclododecenyl group.

Examples of the monovalent aromatic hydrocarbon group include:
aryl groups such as a phenyl group, a tolyl group, a xylyl group, a naphthyl group, and an anthryl group; and
aralkyl groups such as a benzyl group, a phenethyl group, a phenylpropyl group, and a naphthylmethyl group.

In the formula (3), L¹ and L² are each independently a single bond or a divalent organic group.

Examples of the divalent organic group include a group obtained by removing one hydrogen atom from the monovalent hydrocarbon group as R³.

In the formula (3), Z is a divalent group represented by —C(R⁴)₂— or —CO—.

R⁴ in Z is independently at each occurrence a hydrogen atom, a monovalent organic group, a fluorine atom, or a hydroxy group.

Examples of the monovalent organic group include the monovalent hydrocarbon group as R³.

In the formula (3), q is an integer of 0 to 1.

In the formula (3), p is an integer of 0 to (6−m−m′).

In addition, the compound A is preferably, for example, a compound represented by the following formula (4-1), (4-2), or (4-3).

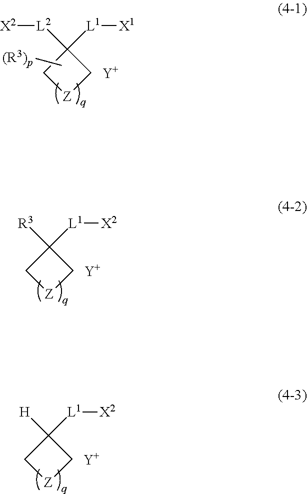

wherein
L¹, L², X¹, X², R³, Y⁺, Z, m, m′, p, and q are the same as those in the formula (3).

The compound A includes the anion moiety described above and the cation moiety described above. More specifically, the compound A may be, for example, a compound including any of the anion moieties described later and any of the cation moieties described later.

More specific examples of the anion moiety include the anions shown below.

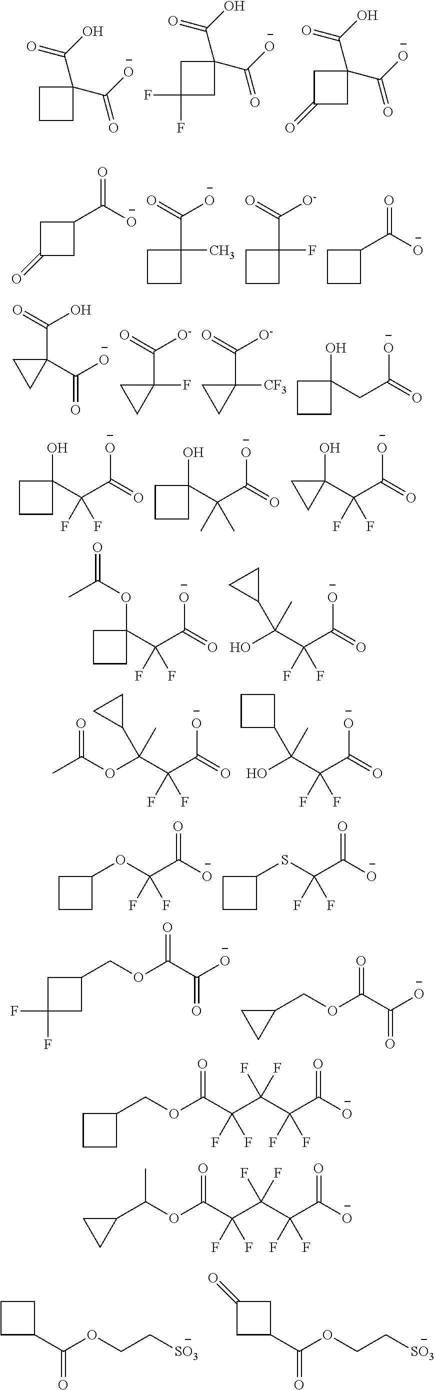

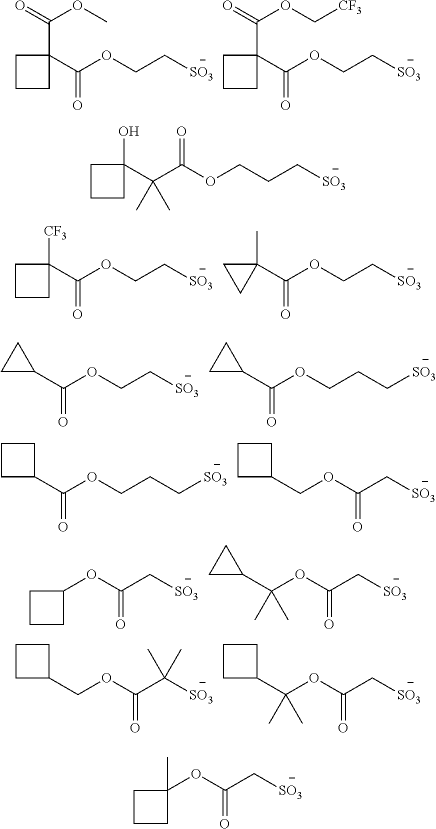

More specific examples of the cation moiety include the cations shown below.

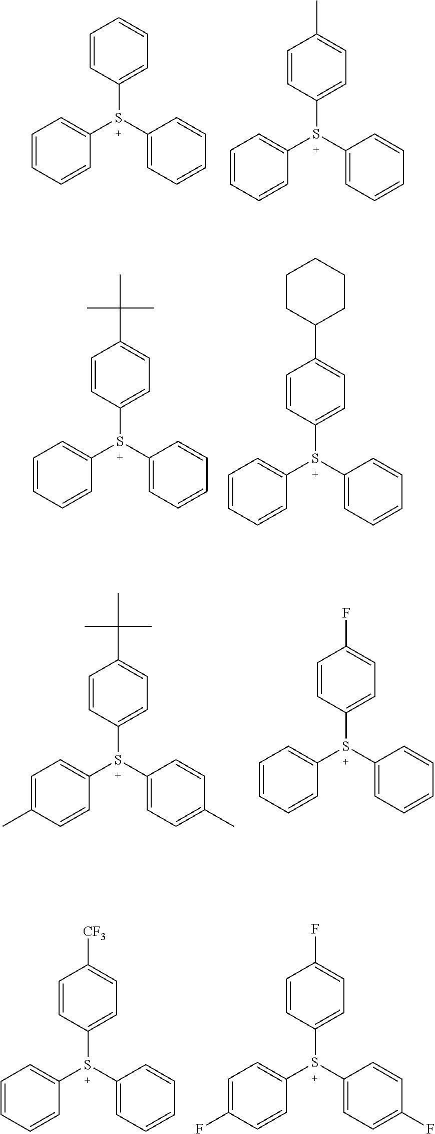

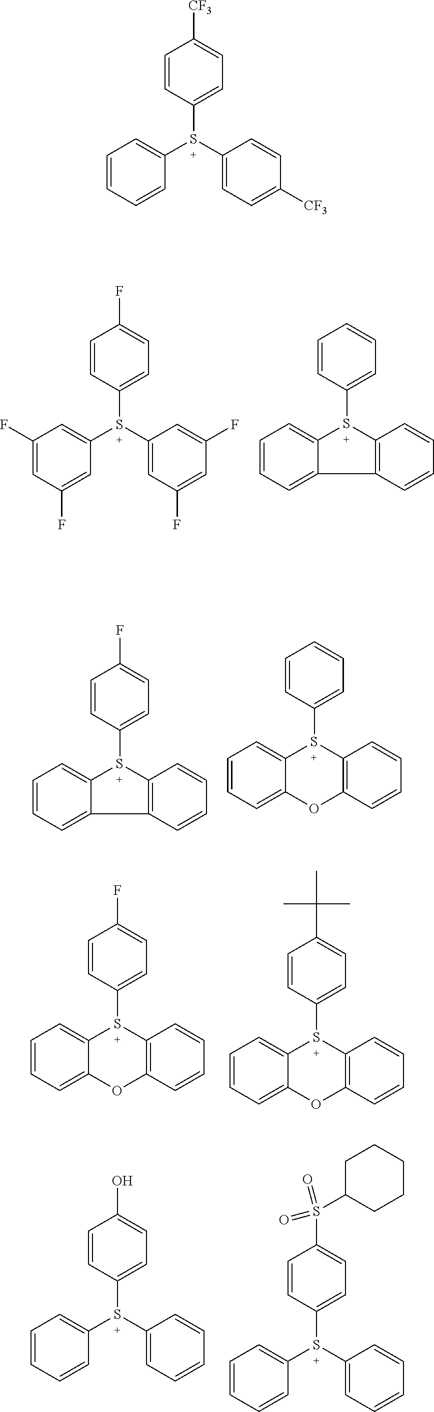

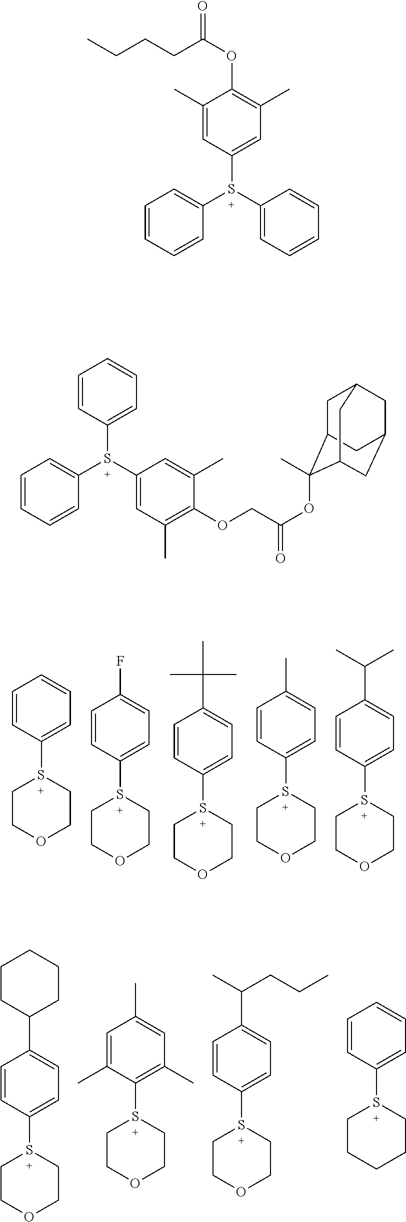

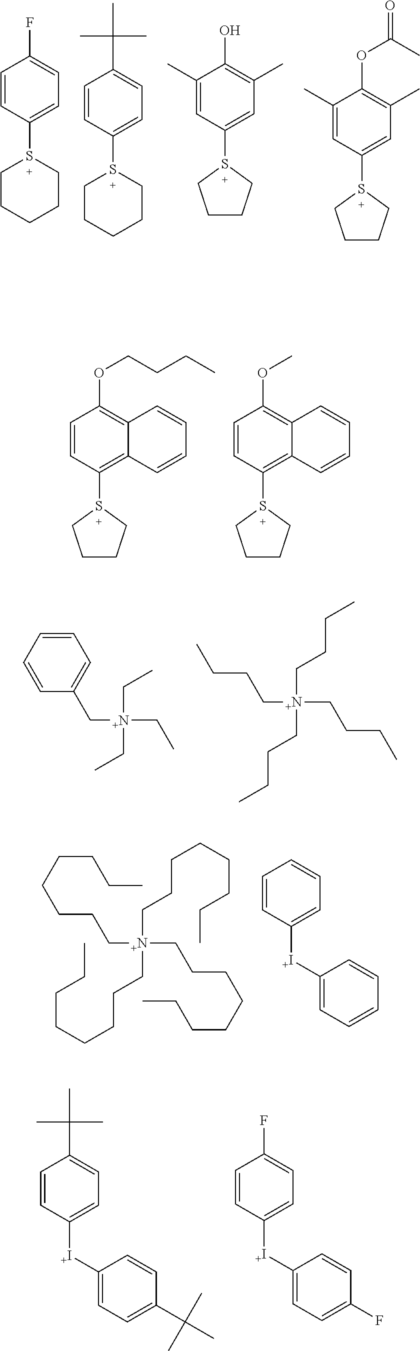

Examples of the compound A more specifically include compounds represented by the following formulas (A-1) to (A-21) (hereinafter, also referred to as “compounds (A-1) to (A-21)”).

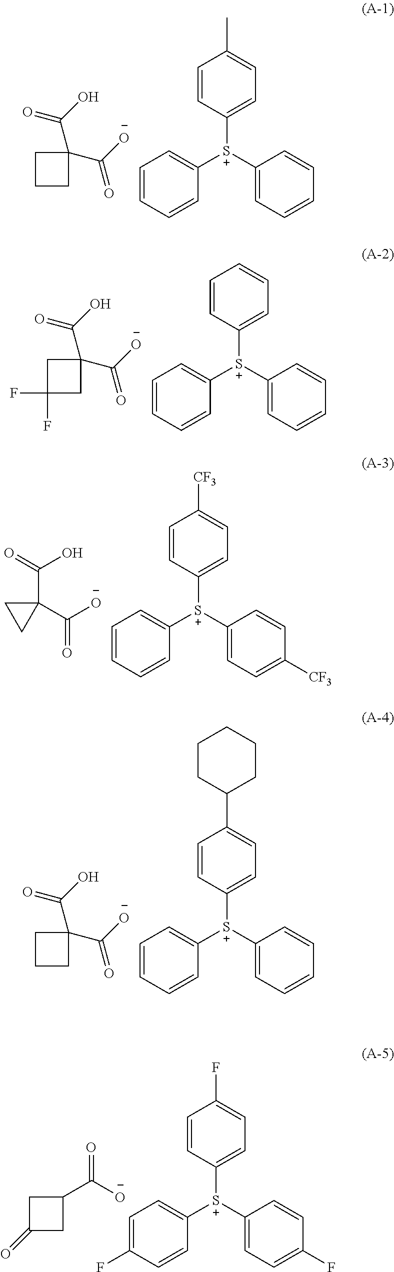

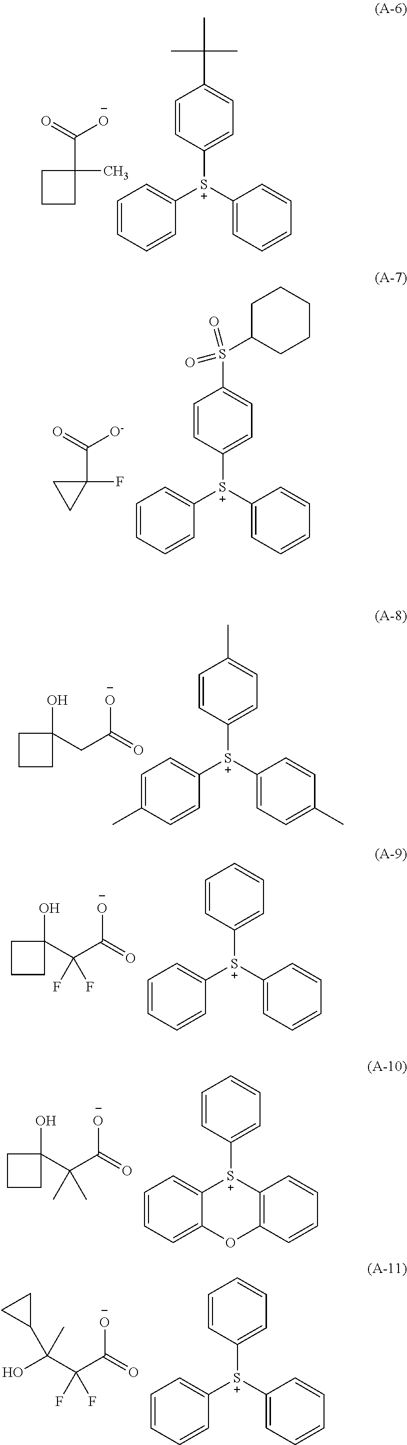

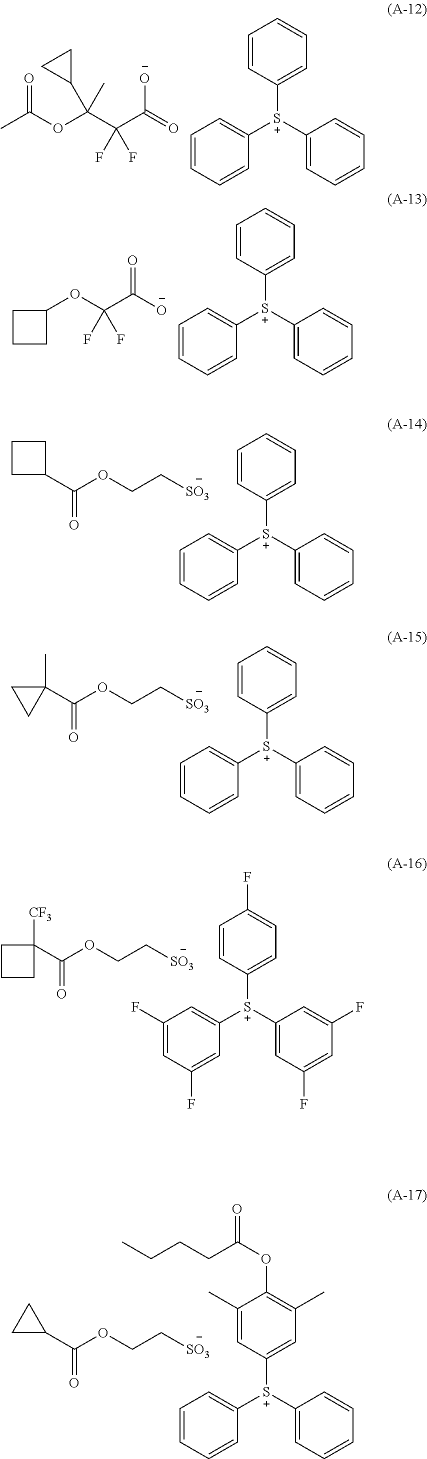

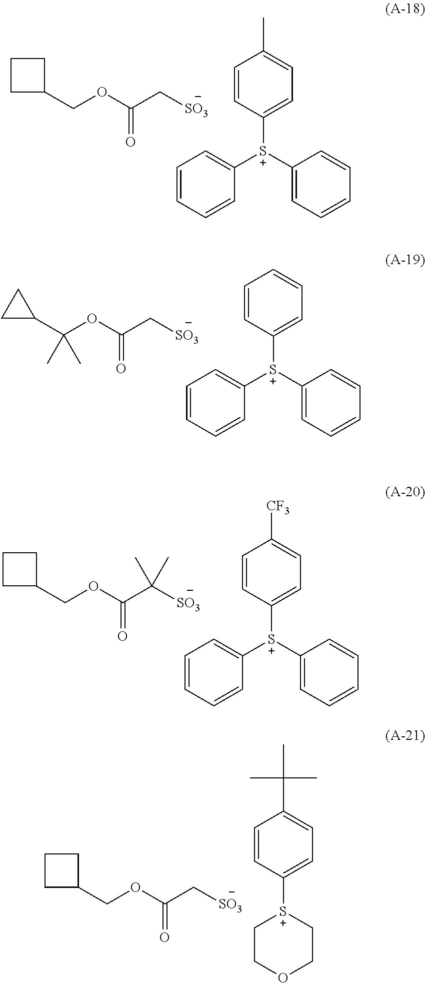

The lower limit of the content of the compound A is preferably 0.1% by mass, more preferably 0.5% by mass, still more preferably 1% by mass, and particularly preferably 3% by mass, based on the entire solid in the radiation-sensitive resin composition. The upper limit of the content is preferably 30% by mass, more preferably 20% by mass, still more preferably 15% by mass, and particularly preferably 10% by mass in the entire solid. When the content of the compound A is less than the above lower limit, lithographic performance such as resolution of the radiation-sensitive resin composition may deteriorate. When the content of the compound A exceeds the above upper limit, the sensitivity of the radiation-sensitive resin composition may deteriorate. Here, the “entire solid” refers to the components other than the solvent of the radiation-sensitive resin composition.

The lower limit of the content of the compound A is preferably 0.1 parts by mass, more preferably 0.5 parts by mass, still more preferably 1 part by mass, and particularly preferably 3 parts by mass per 100 parts by mass of the base resin B described later. The upper limit of the content of the compound A is preferably 30 parts by mass, more preferably 20 parts by mass, still more preferably 15 parts by mass, and particularly preferably 10 parts by mass per 100 parts by mass of the base resin B.

### <Resin>

The resin (resin B) is an assembly of polymers containing a structural unit having an acid-dissociable group (this structural unit is hereinafter also referred to as “structural unit (I)”) (this resin is hereinafter also referred to as “base resin”). The “acid-dissociable group” refers to a group that substitutes for a hydrogen atom of a carboxy group, a phenolic hydroxy group, an alcoholic hydroxy group, a sulfo group, or the like and is dissociated by the action of an acid. The radiation-sensitive resin composition is superior in patternability because the resin has the structural unit (I).

In addition to the structural unit (I), the base resin preferably contains a structural unit (II) containing at least one selected from the group consisting of a lactone structure, a cyclic carbonate structure, and a sultone structure, which are described later, and may contain other structural units than the structural units (I) and (II). Each of the structural units will be described below.

### [Structural Unit (I)]

The structural unit (I) is a structural unit containing an acid-dissociable group. The structural unit (I) is not particularly limited as long as it contains an acid-dissociable group, and examples thereof include a structural unit having a tertiary alkyl ester moiety, a structural unit having a structure in which a hydrogen atom of a phenolic hydroxy group is substituted with a tertiary alkyl group, and a structural unit having an acetal bond. From the viewpoint of improving the patternability of the radiation-sensitive resin composition, a structural unit represented by the following formula (6) (hereinafter also referred to as “structural unit (I-1)”) is preferable.

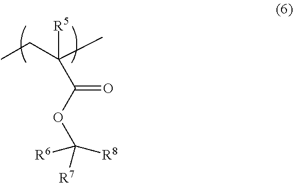

wherein
R⁵ is a hydrogen atom, a fluorine atom, a methyl group, or a trifluoromethyl group,
R⁶ is a monovalent hydrocarbon group having 1 to 20 carbon atoms, and
R⁷ and R⁶ each independently represent a monovalent chain hydrocarbon group having 1 to 10 carbon atoms or a monovalent alicyclic hydrocarbon group having 3 to 20 carbon atoms, or a divalent alicyclic group having 3 to 20 carbon atoms in which R⁷ and R⁸ are combined with each other and which is constituted by R⁷ and R⁸ together with the carbon atoms to which these groups are bonded.

From the viewpoint of the copolymerizability of a monomer that affords the structural unit (I-1), a hydrogen atom and a methyl group are preferable as R⁵, and a methyl group is more preferable.

Examples of the monovalent hydrocarbon group having 1 to 20 carbon atoms represented by R⁶ include a chain hydrocarbon group having 1 to 10 carbon atoms, a monovalent alicyclic hydrocarbon group having 3 to 20 carbon atoms, and a monovalent aromatic hydrocarbon group having 6 to 20 carbon atoms.

Examples of the chain hydrocarbon groups having 1 to 10 carbon atoms represented by R⁶ to R⁸ include a linear or branched saturated hydrocarbon group having 1 to 10 carbon atoms and a linear or branched unsaturated hydrocarbon group having 1 to 10 carbon atoms.

Examples of the alicyclic hydrocarbon groups having 3 to 20 carbon atoms represented by R⁶ to R⁸ include monocyclic or polycyclic saturated hydrocarbon groups having 3 to 20 carbon atoms and monocyclic or polycyclic unsaturated hydrocarbon groups having 3 to 20 carbon atoms. Preferred examples of the monocyclic saturated hydrocarbon groups include a cyclopentyl group, a cyclohexyl group, a cycloheptyl group, and a cyclooctyl group. Preferred examples of the polycyclic cycloalkyl group include bridged alicyclic hydrocarbon groups such as a norbornyl group, an adamantyl group, a tricyclodecyl group, and a tetracyclododecyl group. The bridged alicyclic hydrocarbon group refers to a polycyclic alicyclic hydrocarbon group in which two carbon atoms that constitute an alicyclic ring and not adjacent to each other are bonded by a bonding chain containing one or more carbon atoms.

Examples of the monovalent aromatic hydrocarbon groups having 6 to 20 carbon atoms represented by R⁶ include:
aryl groups such as a phenyl group, a tolyl group, a xylyl group, a naphthyl group, and an anthryl group; and
aralkyl groups such as a benzyl group, a phenethyl group, and a naphthylmethyl group.

R⁶ is preferably a linear or branched saturated hydrocarbon group having 1 to 10 carbon atoms or an alicyclic hydrocarbon group having 3 to 20 carbon atoms.

The divalent alicyclic group having 3 to 20 carbon atoms in which the chain hydrocarbon group or alicyclic hydrocarbon group represented by R⁷ and that represented by R⁸ are combined with each other and which is constituted by R⁷ and R⁸ and the carbon atoms to which these groups are bonded is not particularly limited as long as it is a group obtained by removing two hydrogen atoms from the same carbon atom contained in the carbon ring of the monocyclic or polycyclic alicyclic hydrocarbon having the aforementioned number of carbon atoms. The group may be either a monocyclic hydrocarbon group or a polycyclic hydrocarbon group, and the polycyclic hydrocarbon group may be either a bridged alicyclic hydrocarbon group or a fused alicyclic hydrocarbon group, and may be either a saturated hydrocarbon group or an unsaturated hydrocarbon group. It is to be noted that the condensed alicyclic hydrocarbon group refers to a polycyclic alicyclic hydrocarbon group in which two or more alicyclic rings share their sides (bond between two adjacent carbon atoms).

Among the monocyclic alicyclic hydrocarbon groups, a cyclopentanediyl group, a cyclohexanediyl group, a cycloheptanediyl group, a cyclooctanediyl group, and the like are preferable as the saturated hydrocarbon group, and a cyclopentenediyl group, a cyclohexenediyl group, a cycloheptenediyl group, a cyclooctenediyl group, a cyclodecenediyl group, and the like are preferable as the unsaturated hydrocarbon group. As the polycyclic alicyclic hydrocarbon group, bridged alicyclic saturated hydrocarbon groups are preferable, and for example, a bicyclo[2.2.1]heptane-2,2-diyl group (norbornane-2,2-diyl group), a bicyclo[2.2.2]octane-2,2-diyl group, and a tricyclo[3.3.1.1^{3,7}]decane-2,2-diyl group (adamantane-2,2-diyl group) are preferable.

Among them, it is preferable that R⁶ is an alkyl group having 1 to 4 carbon atoms and the alicyclic structure in which R⁷ and R⁸ are combined with each other and which is constituted by R⁷ and R⁶ together with the carbon atoms to which these groups are bonded is a polycyclic or monocyclic cycloalkane structure.

Examples of the structural unit (I-1) include structural units represented by the following formulas (6-1) to (6-6) (hereinafter also referred to as “structural units (I-1-1) to (I-1-6)”).

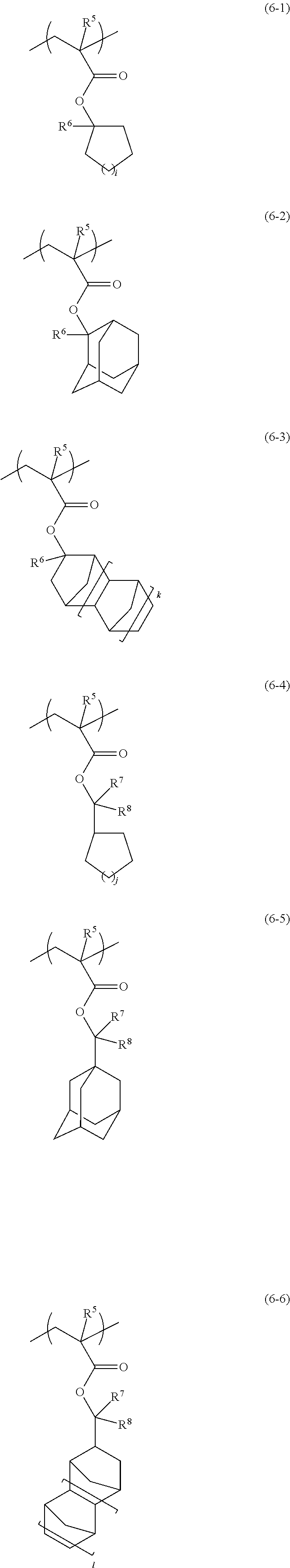

In the above formulas (6-1) to (6-6), R⁵ to R⁸ have the same definitions as those in the above formula (6). i and j are each independently an integer of 1 to 4. k and 1 are 0 or 1.

i and j are preferably 1. R⁶ is preferably a methyl group, an ethyl group, an isopropyl group, or a cyclopentyl group. R⁷ and R⁸ are preferably a methyl group or an ethyl group.

The base resin may contain one type of the structural unit (I) or two or more types of the structural unit (I) in combination.

The lower limit of the content ratio of the structural unit (I) (a total content ratio when a plurality of types are contained) is preferably 10 mol %, more preferably 20 mol %, still more preferably 30 mol %, and particularly preferably 35 mol % based on all structural units constituting the base resin. The upper limit of the content ratio is preferably 80 mol %, more preferably 75 mol %, still more preferably 70 mol %, and particularly preferably 65 mol %. When the content ratio of the structural unit (I) is adjusted to within the above range, the patternability of the radiation-sensitive resin composition can be further improved.

### [Structural Unit (II)]

The structural unit (II) is a structural unit containing at least one structure selected from the group consisting of a lactone structure, a cyclic carbonate structure, and a sultone structure. When the base resin further has the structural unit (II), the solubility of the base resin in a developer can be adjusted, and as a result, the lithographic performance, such as resolution, of the radiation-sensitive resin composition can be improved. In addition, the adhesion between a resist pattern formed from the base resin and a substrate can be improved.

Examples of the structural unit (II) include structural units represented by the following formulas (T-1) to (T-10).

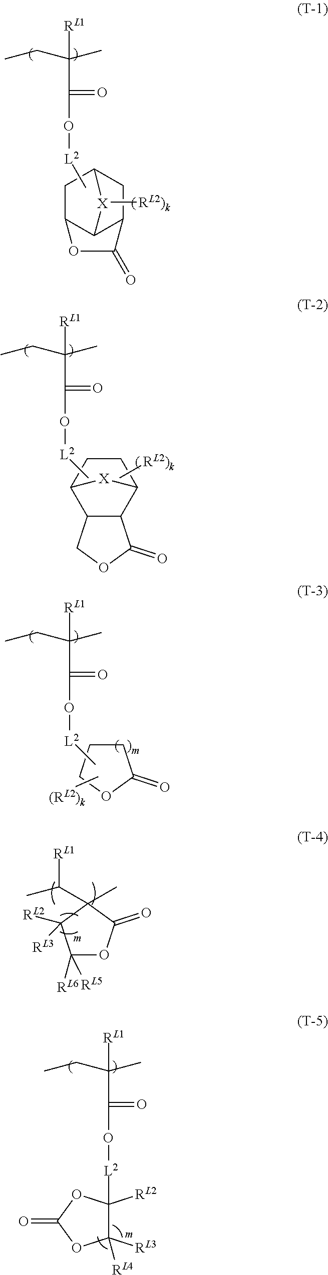

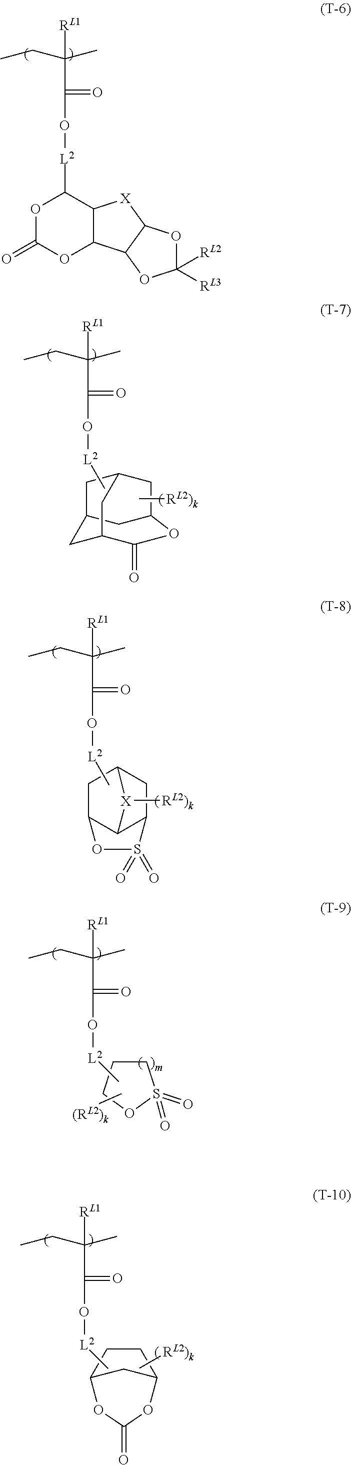

In the above formula, R^{L1} is a hydrogen atom, a fluorine atom, a methyl group, or a trifluoromethyl group. R^{L2} to R^{L5} are each independently a hydrogen atom, an alkyl group having 1 to 4 carbon atoms, a cyano group, a trifluoromethyl group, a methoxy group, a methoxycarbonyl group, a hydroxy group, a hydroxymethyl group, or a dimethylamino group. R^{L4} and R^{L5} may be combined with each other and constitute a divalent alicyclic group having 3 to 8 carbon atoms together with the carbon atom to which they are bonded. L² is a single bond or a divalent linking group. X is an oxygen atom or a methylene group. k is an integer of 0 to 3. m is an integer of 1 to 3.

Examples of the divalent alicyclic group having 3 to 8 carbon atoms in which R^{L4} and R^{L5} are combined with each other and which is constituted by R^{L4} and R^{L5} together with the carbon atoms to which these groups are bonded include groups having 3 to 8 carbon atoms among divalent alicyclic groups having 3 to 20 carbon atoms in which the chain hydrocarbon group or alicyclic hydrocarbon group represented by R¹⁹ in the above formula (3) and that represented by R²⁰ are combined with each other and which is constituted by R¹⁹ and R²⁰ together with the carbon atoms to which these groups are bonded. One or more hydrogen atoms on the alicyclic group may be replaced by a hydroxy group.

Examples of the divalent linking group represented by L² include a divalent linear or branched hydrocarbon group having 1 to 10 carbon atoms, a divalent alicyclic hydrocarbon group having 4 to 12 carbon atoms, and a group composed of one or more among these hydrocarbon groups and at least one group among —CO—, —O—, —NH—, and —S.

Among them, the structural unit (II) is preferably a structural unit containing a lactone structure, more preferably a structural unit containing a norbornane lactone structure, and still more preferably a structural unit derived from norbornane lactone-yl (meth)acrylate.

The lower limit of the content ratio of the structural unit (II) is preferably 20 mol %, more preferably 30 mol %, and still more preferably 35 mol % based on all structural units constituting the base resin. The upper limit of the content ratio is preferably 75 mol %, more preferably 70 mol %, and still more preferably 65 mol %. When the content ratio of the structural unit (II) is adjusted to within the above range, the lithographic performance, such as resolution, of the radiation-sensitive resin composition and the adhesion between a resist patter to be formed and a substrate can be further improved.

### [Structural Unit (III)]

The base resin optionally has other structural units in addition to the structural units (I) and (II). Examples of the other structural units include a structural unit (III) containing a polar group (excluding those corresponding to the structural unit (II)). When the base resin further has the structural unit (III), the solubility of the base resin in a developer can be adjusted, and as a result, the lithographic performance, such as resolution, of the radiation-sensitive resin composition can be improved. Examples of the polar group include a hydroxy group, a carboxy group, a cyano group, a nitro group, and a sulfonamide group. Among them, a hydroxy group and a carboxy group are preferable, and a hydroxy group is more preferable.

Examples of the structural unit (III) include structural units represented by the following formulas.

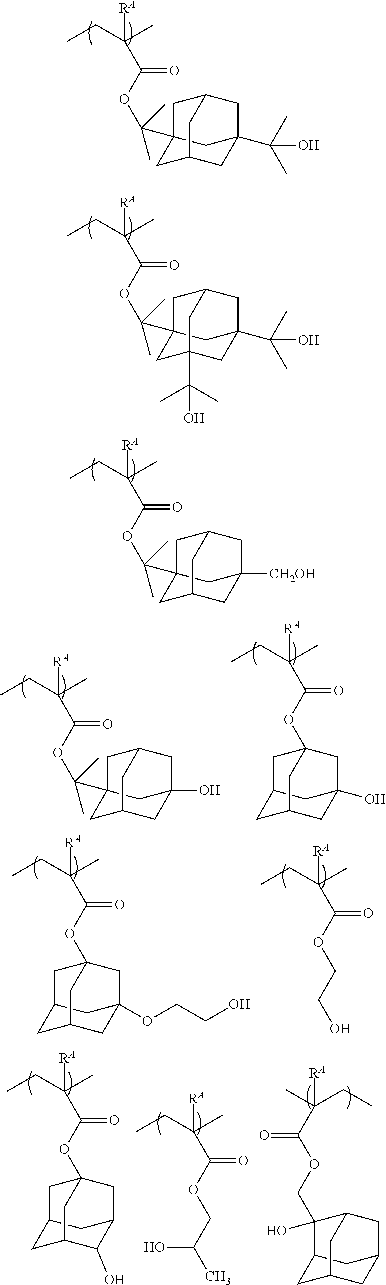

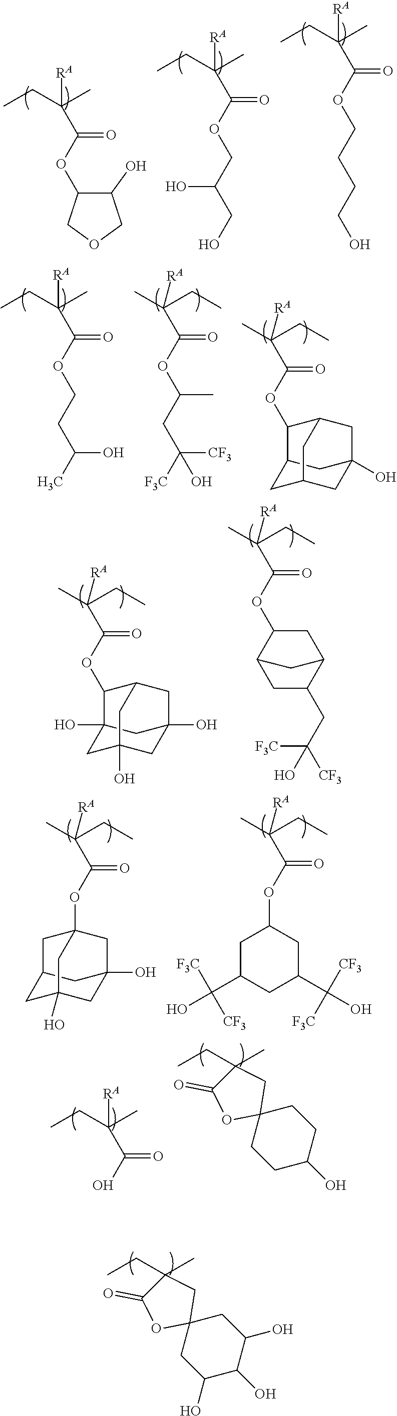

In the above formulas, R^{A} is a hydrogen atom, a fluorine atom, a methyl group, or a trifluoromethyl group.

When the base resin has the structural unit (III) having a polar group, the lower limit of the content ratio of the structural unit (III) is preferably 5 mol %, more preferably 8 mol %, and still more preferably 10 mol % based on all structural units constituting the base resin. The upper limit of the content ratio is preferably 40 mol %, more preferably 35 mol %, and still more preferably 30 mol %. When the content ratio of the structural unit (III) is adjusted to within the above range, the lithographic performance, such as resolution, of the radiation-sensitive resin composition can be further improved.

### [Structural Unit (IV)]

Besides the structural unit (III) having a polar group, the base resin optionally has, as other structural units, a structural unit derived from hydroxystyrene or a structural unit having a phenolic hydroxy group (hereinafter, both are also collectively referred to as “structural unit (IV)”). The structural unit (IV) contributes to improvement of etching resistance and improvement of a difference in solubility of a developer (dissolution contrast) between an exposed area and an unexposed area. In particular, the structural unit (IV) can be suitably applied to pattern formation using exposure with a radiation having a wavelength of 50 nm or less, such as an electron beam or EUV. In this case, the resin preferably has the structural unit (I) together with the structural unit (IV).

The structural unit derived from hydroxystyrene is represented by, for example, the following formulas (7-1) to (7-2), and the structural unit having a phenolic hydroxy group is represented by, for example, the following formulas (7-3) to (7-4).

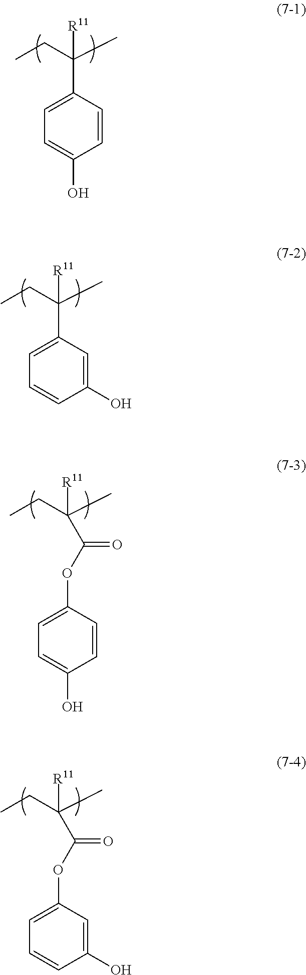

In the above formulas (7-1) to (7-4), R¹¹ is a hydrogen atom, a fluorine atom, a methyl group, or a trifluoromethyl group.

When the structural unit (IV) is obtained, it is preferable to obtain the structural unit (IV) by polymerizing the monomer in a state where the phenolic hydroxy group is protected by a protecting group such as an alkali-dissociable group (e.g., an acyl group) during polymerization, and then deprotecting the polymerized product by hydrolysis.

In the case of a resin for exposure to radiation having a wavelength of 50 nm or less, the lower limit of the content ratio of the structural unit (IV) is preferably 10 mol %, and more preferably 20 mol % based on all structural units constituting the resin. In addition, the upper limit of the content ratio is preferably 70 mol %, and more preferably 60 mol %.

### (Method for Synthesizing Base Resin)

The base resin can be synthesized by, for example, polymerizing monomers that will afford respective structural units in an appropriate solvent using a radical polymerization initiator or the like.

Examples of the radical polymerization initiator include azo radical initiators, such as azobisisobutyronitrile (AIBN), 2,2′-azobis(4-methoxy-2,4-dimethylvaleronitrile), 2,2′-azobis(2-cyclopropylpropionitrile), 2,2′-azobis(2,4-dimethylvaleronitrile) and dimethyl 2,2′-azobisisobutyrate; and peroxide radical initiators, such as benzoyl peroxide, t-butyl hydroperoxide and cumene hydroperoxide. Among them, AIBN and dimethyl 2,2′-azobisisobutyrate are preferable, and AIBN is more preferable. These radical initiators may be used singly, or two or more of them may be used in combination.

Examples of the solvent to be used in the polymerization include:
alkanes such as n-pentane, n-hexane, n-heptane, n-octane, n-nonane, and n-decane;
cycloalkanes such as cyclohexane, cycloheptane, cyclooctane, decalin, and norbornane;
aromatic hydrocarbons such as benzene, toluene, xylene, ethylbenzene, and cumene;
halogenated hydrocarbons such as chlorobutanes, bromohexanes, dichloroethanes, hexamethylene dibromide, and chlorobenzene;
saturated carboxylic acid esters such as ethyl acetate, n-butyl acetate, i-butyl acetate, and methyl propionate;
ketones such as acetone, methyl ethyl ketone, 2-butanone, 4-methyl-2-pentanone, and 2-heptanone;
ethers such as tetrahydrofuran, dimethoxyethanes, and diethoxyethanes; and
alcohols such as methanol, ethanol, 1-propanol, 2-propanol, and 4-methyl-2-pentanol. The solvents to be used for the polymerization may be used singly, or two or more solvents may be used in combination.

The reaction temperature in the polymerization is usually 40° C. to 150° C., and preferably 50° C. to 120° C. The reaction time is usually 1 hour to 48 hours, and preferably 1 hour to 24 hours.

The molecular weight of the base resin is not particularly limited, and the lower limit of the standard polystyrene weight average molecular weight (Mw) as determined by Gel Permeation Chromatography (GPC) is preferably 1,000, more preferably 2,000, still more preferably 3,000, and particularly preferably 4,000. The upper limit of the Mw is preferably 50,000, more preferably 30,000, still more preferably 15,000, and particularly preferably 12,000. When the Mw of the base resin is less than the lower limit, the heat resistance of the resulting resist film may be deteriorated. When the Mw of the base resin exceeds the above upper limit, the developability of the resist film may be deteriorated.

The ratio (Mw/Mn) of Mw to the number average molecular weight (Mn) of the base resin as determined by GPC relative to standard polystyrene is usually 1 or more and 5 or less, preferably 1 or more and 3 or less, and more preferably 1 or more and 2 or less.

The Mw and the Mn of a resin in the present description are values measured using gel permeation chromatography (GPC) under the following conditions.
GPC column: two G2000HXL, one G3000HXL, one G4000HXL (all manufactured by Tosoh Corporation)
Column temperature: 40° C.
Elution solvent: tetrahydrofuran
Flow rate: 1.0 mL/min
Sample concentration: 1.0% by mass
Amount of sample injected: 100 μL
Detector: differential refractometer
Standard substance: monodisperse polystyrene

The content ratio of the base resin is preferably 70% by mass or more, more preferably 80% by mass or more, and still more preferably 85% by mass or more based on the entire solid of the radiation-sensitive resin composition.

### (Other Resins)

The radiation-sensitive resin composition of the present embodiment may contain a resin having a higher content rate by mass of fluorine atoms than the base resin as described above (hereinafter also referred as “high fluorine-containing resin”) as other resin. When the radiation-sensitive resin composition contains the high fluorine-containing resin, the high fluorine-containing resin can be localized in the surface layer of a resist film compared to the base resin, and as a result, the water repellency of the surface of the resist film can be further enhanced in the case of immersion exposure.

The high fluorine-containing resin preferably has, for example, a structural unit represented by the following formula (8) (hereinafter also referred to as “structural unit (V)), and as necessary, may have the structural unit (I) or the structural unit (III) in the base resin.

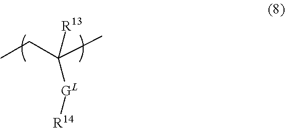

In the above formula (8), R¹³ is a hydrogen atom, a methyl group, or a trifluoromethyl group. G^{L} is a single bond, an oxygen atom, a sulfur atom, —COO—, —SO₂ONH—, —CONH—, or —OCONH—. R¹⁴ is a monovalent fluorinated chain hydrocarbon group having 1 to 20 carbon atoms or a monovalent fluorinated alicyclic hydrocarbon group having 3 to 20 carbon atoms.

As R¹³, a hydrogen atom and a methyl group are preferable from the viewpoint of the copolymerizability of a monomer that affords the structural unit (V), and a methyl group is more preferable.

As the G^{L}, a single bond and —COO— are preferable from the viewpoint of the copolymerizability of a monomer that affords the structural unit (V), and —COO— is more preferable.

Examples of the monovalent fluorinated chain hydrocarbon group having 1 to 20 carbon atoms represented by R¹⁴ include groups in which some or all of the hydrogen atoms in the linear or branched chain alkyl group having 1 to 20 carbon atoms are substituted with fluorine atoms.

Examples of the monovalent fluorinated alicyclic hydrocarbon group having 3 to 20 carbon atoms represented by R¹⁴ include monovalent fluorinated alicyclic hydrocarbon groups having 3 to 20 carbon atoms in which some or all of the hydrogen atoms of a mono- or polycyclic hydrocarbon group are substituted with fluorine atoms.

As the R¹⁴, fluorinated chain hydrocarbon groups are preferable, fluorinated alkyl groups are more preferable, and 2,2,2-trifluoroethyl group, 1,1,1,3,3,3-hexafluoropropyl group, and 5,5,5-trifluoro-1,1-diethylpentyl group is even more preferable.

When the high fluorine-containing resin contains the structural unit (V), the content ratio of the structural unit (V) is preferably 30 mol % or more, more preferably 40 mol % or more, still more preferably 45 mol % or more, and particularly preferably 50 mol % or more based on all structural units constituting the high fluorine-containing resin. The content ratio is preferably 90 mol % or less, more preferably 85 mol % or less, and still more preferably 80 mol % or less. When the content ratio of the structural unit (V) is adjusted to within the above range, the content rate by mass of fluorine atoms in the high fluorine-containing resin can more appropriately be adjusted and the localization in the surface layer of a resist film can be further promoted, and as a result, the water repellency of the resist film at the time of immersion exposure can be further enhanced.

The high fluorine-containing resin may have a fluorine atom-containing structural unit represented by the following formula (f-2) (hereinafter also referred to as structural unit (VI)) in addition to the structural unit (V) or instead of the structural unit (V). When the high fluorine-containing resin has the structural unit (f-2), solubility in an alkaline developer is improved, and the occurrence of development defects can be suppressed.

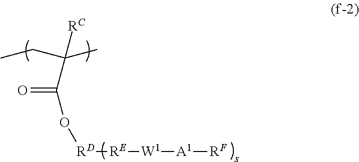

The structural unit (VI) is roughly divided into two cases: a case where it has (x) an alkali-soluble group, and a case where it has (y) a group that is dissociated by the action of an alkali to increase the solubility in an alkaline developer (hereinafter, also simply referred to as “alkali-dissociable group”). Commonly in (x) and (y), in the above formula (f-2), R^{C} is a hydrogen atom, a fluorine atom, a methyl group, or a trifluoromethyl group. R^{D} is a single bond, a hydrocarbon group having 1 to 20 carbon atoms with the valency of (s+1), a structure in which an oxygen atom, a sulfur atom, —NR^{dd}—, a carbonyl group, —COO— or —CONH— is connected to the terminal on R^{E} side of the hydrocarbon group, or a structure in which some of the hydrogen atoms in the hydrocarbon group are substituted with organic groups having a hetero atom. R^{dd} is a hydrogen atom or a monovalent hydrocarbon group having 1 to 10 carbon atoms. s is an integer of 1 to 3.

When the structural unit (VI) has (x) an alkali-soluble group, R^{F} is a hydrogen atom, and A¹ is an oxygen atom, —COO—* or —SO₂O—*. * indicates a site that bonds to R^{F}. W¹ is a single bond, a hydrocarbon group having 1 to 20 carbon atoms, or a divalent fluorinated hydrocarbon group. When A¹ is an oxygen atom, W¹ is a fluorinated hydrocarbon group having a fluorine atom or a fluoroalkyl group on the carbon atom to which A¹ is bonded. R^{E} is a single bond or a divalent organic group having 1 to 20 carbon atoms. When s is 2 or 3, a plurality of R^{E}'s, W¹'s, A¹'s, and R^{F}'s may be the same or different, respectively. When the structural unit (VI) has (x) an alkali-soluble group, affinity to an alkaline developer can be increased, and development defects can be suppressed. As the structural unit (VI) having (x) an alkali-soluble group, a case where A¹ is an oxygen atom and W¹ is a 1,1,1,3,3,3-hexafluoro-2,2-methanediyl group is particularly preferable.

When the structural unit (VI) has (y) an alkali-dissociable group, R^{F} is a monovalent organic group having 1 to 30 carbon atoms, and A¹ is an oxygen atom, —NR^{aa}, —COO—* or —SO₂O—*. R^{aa} is a hydrogen atom or a monovalent hydrocarbon group having 1 to 10 carbon atoms. * indicates a site that bonds to R^{F}. W¹ is a single bond or a divalent fluorinated hydrocarbon group having 1 to 20 carbon atoms. R^{E} is a single bond or a divalent organic group having 1 to 20 carbon atoms. When A¹ is —COO—* or —SO₂O—*, W¹ or R^{F} has a fluorine atom on a carbon atom bonded to A¹ or on a carbon atom adjacent thereto. When A¹ is an oxygen atom, W¹ and R^{E} are single bonds, R^{D} is a structure in which a carbonyl group is bonded to a terminal on the R^{E} side of a hydrocarbon group having 1 to 20 carbon atoms, and R^{F} is an organic group having a fluorine atom. When s is 2 or 3, a plurality of R^{E}'s, W¹'s, A¹'s, and R^{F}'s may be the same or different, respectively. When the structural unit (VI) has (y) an alkali-dissociable group, the surface of a resist film changes from hydrophobic to hydrophilic in an alkali development step. As a result, the affinity to a developer can be greatly increased, and development defects can be more efficiently suppressed. As the structural unit (VI) having (y) an alkali-dissociable group, a structural unit in which A¹ is —COO—*, and R^{F}, W¹, or both of them have a fluorine atom is particularly preferable.

As R^{C}, a hydrogen atom and a methyl group are preferable from the viewpoint of the copolymerizability of a monomer that affords the structural unit (VI), and a methyl group is more preferable.

When R^{E} is a divalent organic group, a group having a lactone structure is preferable, a group having a polycyclic lactone structure is more preferable, and a group having a norbornanelactone structure is still more preferable.

When the high fluorine-containing resin contains the structural unit (VI), the content ratio of the structural unit (VI) is preferably 40 mol % or more, more preferably 50 mol % or more, and still more preferably 60 mol % or more based on all structural units constituting the high fluorine-containing resin. The content ratio is preferably 95 mol % or less, more preferably 90 mol % or less, and still more preferably 85 mol % or less. When the content ratio of the structural unit (VI) is set to fall within the above range, water repellency of a resist film during immersion exposure can further be improved.

### [Other Structural Unit]

A high fluorine-content resin may contain a structural unit having an alicyclic structure represented by the following formula (9) as a structural unit other than the structural units listed above,

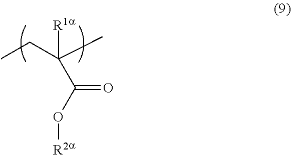

in the formula (9), R^{1α} represents a hydrogen atom, a fluorine atom, a methyl group, or a trifluoromethyl group, and R^{2α} is a monovalent alicyclic hydrocarbon group having 3 to 20 carbon atoms.)

In the formula (9), as the monovalent alicyclic hydrocarbon group having 3 to 20 carbon atoms represented by R^{2α}, alicyclic hydrocarbon groups having 3 to 20 carbon atoms represented by R⁶ to R⁸ in the formula (6) can be suitably employed.

When the high fluorine-containing resin contains the structural unit having an alicyclic structure, the content ratio of the structural unit having an alicyclic structure is preferably 10 mol % or more, more preferably 20 mol % or more, and still more preferably 30 mol % or more based on all structural units constituting the high fluorine-content resin. The content ratio is preferably 70 mol % or less, more preferably 60 mol % or less, and still more preferably 50 mol % or less.

The lower limit of the Mw of the high fluorine-containing resin is preferably 1,000, more preferably 2,000, still more preferably 3,000, and particularly preferably 5,000. The upper limit of Mw is preferably 50,000, more preferably 30,000, still more preferably 20,000, and particularly preferably 15,000.

The Mw/Mn of the high fluorine-containing resin is usually 1 or more, and more preferably 1.1 or more. The Mw/Mn of the high fluorine-containing resin is usually 5 or less, preferably 3 or less, more preferably 2 or less, and still more preferably 1.9 or less.

The content of the high fluorine-containing resin is preferably 0.1 parts by mass or more, more preferably 0.5 parts by mass or more, still more preferably 1 part by mass or more, and particularly preferably 1.5 parts by mass or more based on 100 parts by mass of the base resin. The content of the high fluorine-containing resin is preferably 15 parts by mass or less, more preferably 10 parts by mass or less, still more preferably 8 parts by mass or less, and particularly preferably 5 parts by mass or less.

When the content of the high fluorine-containing resin is adjusted to within the above range, the high fluorine-containing resin can be more effectively localized in the surface layer of a resist film, and as a result, the water repellency of the surface of the resist film during immersion exposure can be further enhanced. The radiation-sensitive resin composition may contain one high fluorine-containing resin or two or more high fluorine-content resins.

### (Method for Synthesizing High Fluorine-Containing Resin)

The high fluorine-containing resin can be synthesized by the same method as the method for synthesizing a base resin described above.

### <Radiation-Sensitive Acid Generator>

The radiation-sensitive acid generator other than the compound A (hereinafter, also referred to as “radiation-sensitive acid generator”) includes an onium salt compound. The radiation-sensitive acid generator is a substance that generates an acid by exposure to light. The acid-dissociable groups of the resin B and the like are dissociated by the generated acid to generate carboxy groups or the like and, as a result, the solubility of these polymers in a developer changes. Therefore, the inclusion form in the radiation-sensitive resin composition of the radiation-sensitive acid generator that can form a resist pattern from the radiation-sensitive resin composition may be a form of a low molecular weight compound as described later, a form in which the radiation-sensitive acid generator is incorporated as a part of a polymer, or both of these forms.

The radiation-sensitive acid generator may contain an N-sulfonyloxyimide compound, a halogen-containing compound, a diazoketone compound, or the like in addition to the onium salt compound as long as the effect of the present invention is not impaired.

Examples of the onium salt compound include:
a sulfonium salt, a tetrahydrothiophenium salt, an iodonium salt, a phosphonium salt, a diazonium salt, and a pyridinium salt.

Examples of the radiation-sensitive acid generator include the compounds described in paragraphs [0080] to [0113] of JP-A-2009-134088.

The radiation-sensitive acid generator is preferably a compound represented by the following formula (10). It is considered that when the radiation-sensitive acid generator is a compound represented by the following formula (10), the diffusion length of the acid generated by exposure to light in a resist film is more adequately shortened due to the interaction with the polar structure of the resin B. As a result, the lithography performance of the radiation-sensitive resin composition can be further improved.

R^{b1}—R^{b2}—SO₃⁻M⁺ (10)
wherein
R^{b1} is a monovalent group containing an alicyclic structure or a monovalent group containing an aliphatic heterocyclic structure,
R^{b2} is a fluorinated alkanediyl group having 1 to 10 carbon atoms, and
M⁺ is a monovalent radiolytic onium cation.

The number of ring members of the alicyclic structure and the aliphatic heterocyclic structure in R^{b1} is, for example, 3 or more and 20 or less. The “number of ring members” refers to the number of atoms constituting the rings of an alicyclic structure and an aliphatic heterocyclic structure, and in the case of a polycyclic alicyclic structure and a polycyclic aliphatic heterocyclic structure, refers to the number of atoms constituting these polycycles.

Examples of the monovalent group having an alicyclic structure represented by R^{b1} include:
monocyclic cycloalkyl groups such as a cyclopropyl group, a cyclobutyl group, a cyclopentyl group, a cyclohexyl group, a cyclooctyl group, a cyclononyl group, a cyclodecyl group, and a cyclododecyl group;
monocyclic cycloalkenyl groups such as a cyclopropenyl group, a cyclobutenyl group, a cyclopentenyl group, a cyclohexenyl group, a cyclooctenyl group, and a cyclodecenyl group;
polycyclic cycloalkyl groups such as a norbornyl group, an adamantyl group, a tricyclodecyl group, and a tetracyclododecyl group; and
polycyclic cycloalkenyl groups such as a norbornenyl group and a tricyclodecenyl group.

Examples of the monovalent group having an aliphatic heterocyclic structure represented by R^{b1} include:
Groups containing a lactone structure such as a butyrolactone-yl group and a norbornane lactone-yl group; Group comprising a sultone structure, such as a norbornane sulton-yl group;
oxygen atom-containing heterocyclic groups such as an oxacyclopropyl group, an oxacyclobutyl group, an oxacyclopentyl group, an oxacyclohexyl group, an oxacycloheptyl group, and an oxanorbornyl group;
Nitrogen atom-containing heterocyclic groups such as an azacyclopropyl group, an azacyclobutyl group, an azacyclopentyl group, an azacyclohexyl group, an azacycloheptyl group, and a diazabicyclooctan-yl group;
sulfur atom-containing heterocyclic groups such as a thiacyclopropyl group, a thiacyclobutyl group, a thiacyclopentyl group, a thiacyclohexyl group, a thiacycloheptyl group, and a thianorbornyl group.

The number of ring members of the alicyclic structure and the aliphatic heterocyclic structure in the group represented by R^{b1} is preferably 6 or more, more preferably 8 or more, still more preferably 9 to 15, and particularly preferably 10 to 13 from the viewpoint of further appropriately adjusting the diffusion length of the acid.

Among them, monovalent groups containing an alicyclic structure having 9 or more ring members and monovalent groups containing an aliphatic heterocyclic structure having 9 or more ring members are preferable as the R^{b1}, an adamantyl group, a hydroxyadamantyl group, a norbornane lacton-yl group, and a 5-oxo-4-oxatricyclo[4.3.1.13, 8]undecan-yl group are more preferable, and an adamantyl group is still more preferable.

Examples of the fluorinated alkanediyl group having 1 to 10 carbon atoms represented by R^{b2} include groups in which one or more of the hydrogen atoms of an alkanediyl group having 1 to 10 carbon atoms, such as a methanediyl group, an ethanediyl group, and a propanediyl group, are substituted with fluorine atoms.

Among them, fluorinated alkanediyl groups in which a fluorine atom is bonded to a carbon atom adjacent to an SO₃⁻ group are preferable, fluorinated alkanediyl groups in which two fluorine atoms are bonded to a carbon atom adjacent to an SO₃⁻ group are more preferable, and a 1,1-difluoromethanediyl group, a 1,1-difluoroethanediyl group, a 1,1,3,3,3-pentafluoro-1,2-propanediyl group, a 1,1,2,2-tetrafluoroethanediyl group, a 1,1,2,2-tetrafluorobutanediyl group, and a 1,1,2,2-tetrafluorohexanediyl group are still more preferable.

As the monovalent radiolytic onium cation represented by M⁺, those the same as the radiation-sensitive onium cations disclosed as examples of Y⁺ in the formula (1) of the compound A can be used, and examples thereof include a sulfonium cation and an iodonium cation.

Examples of the radiation-sensitive acid generator include compounds represented by the following formulas (vi-1) to (vi-17) (hereinafter, also referred to as “compounds (vi-1) to (vi-17)”).

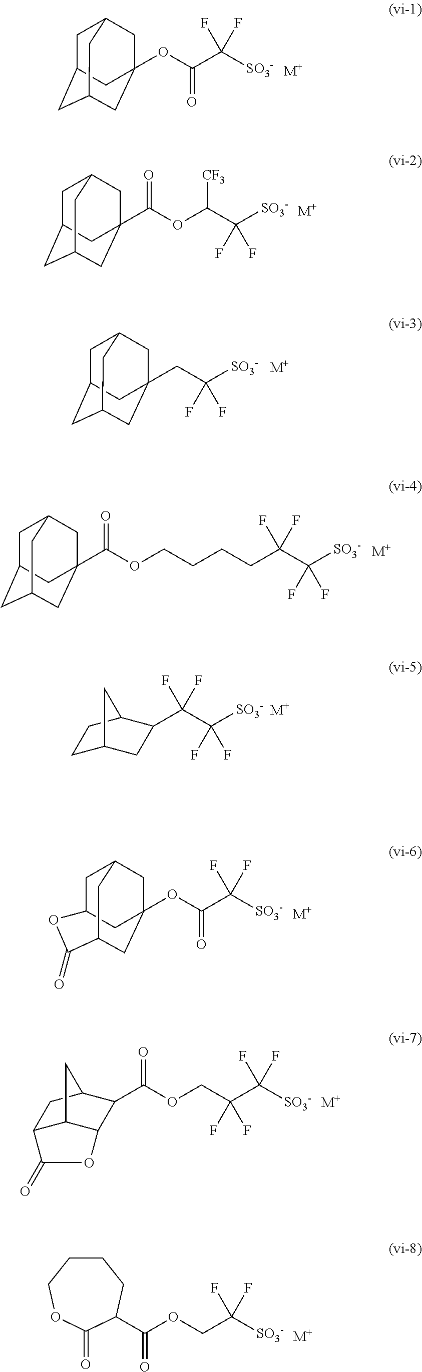

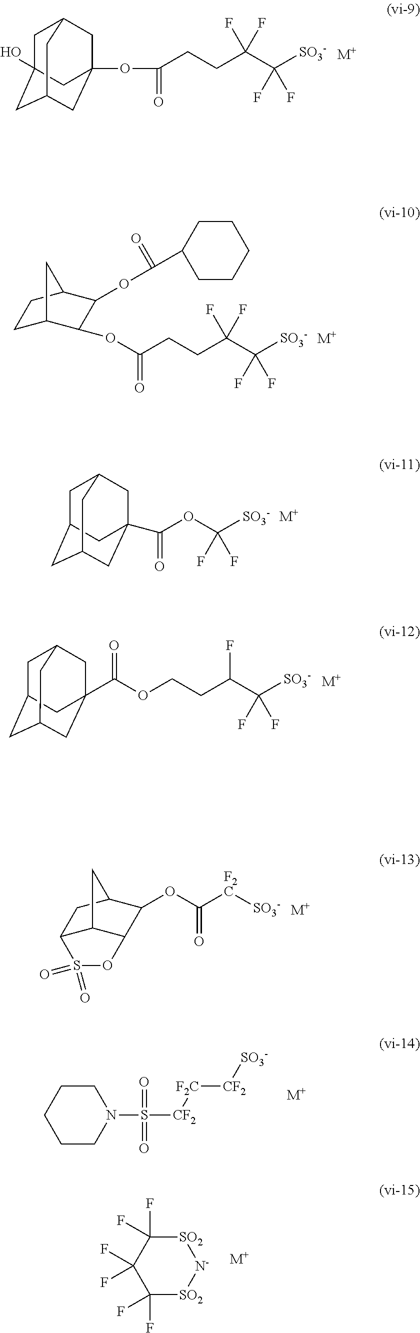

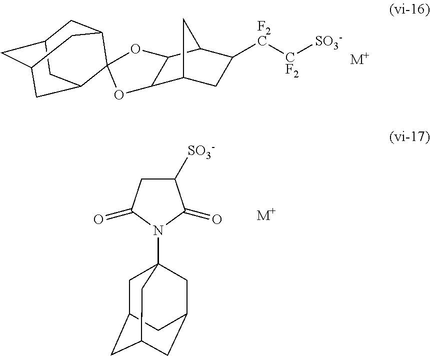

Among them, sulfonium salts are preferable as the radiation-sensitive acid generator, and compounds (vi-1) to (vi-3) and compounds (vi-13) to (vi-17) are more preferable.

The lower limit of the content of the radiation-sensitive acid generator is preferably 2 parts by mass, more preferably 5 parts by mass, and still more preferably 10 parts by mass per 100 parts by mass of the compound A, from the viewpoint of improving the sensitivity and developability of the radiation-sensitive resin composition. The upper limit of the content is preferably 100 parts by mass, more preferably 80 parts by mass, and still more preferably 50 parts by mass.

In addition, the lower limit of the content of the radiation-sensitive acid generator is preferably 0.1 parts by mass, more preferably 0.5 parts by mass, still more preferably 1 part by mass, and particularly preferably 3 parts by mass per 100 parts by mass of the resin B. The upper limit of the content is preferably 30 parts by mass, more preferably 20 parts by mass, and still more preferably 15 parts by mass. The radiation-sensitive acid generator may be used singly or two or more types thereof may be used.

### <Acid Diffusion Controlling Agent>

The radiation-sensitive resin composition may contain an acid diffusion controlling agent other than the compound A (hereinafter, the acid diffusion controlling agent is also referred to as “other acid diffusion controlling agent”), as necessary. Such other acid diffusion controlling agent has the effect of controlling a phenomenon in which an acid generated from the radiation-sensitive acid generator by exposure diffuses in a resist film to prevent an undesired chemical reaction in an unexposed area. In addition, the storage stability of the resulting radiation-sensitive resin composition is improved. The resolution of a resist pattern is further improved, the line width change of a resist pattern due to variation in post exposure delay time between exposure and development treatment can be prevented, and a radiation-sensitive resin composition excellent in process stability can be obtained.

Examples of the other acid diffusion controlling agent include a compound represented by the following formula (11) (hereinafter, also referred to as “nitrogen-containing compound (I)”), a compound having two nitrogen atoms in the same molecule (hereinafter, also referred to as “nitrogen-containing compound (II)”), a compound having three nitrogen atoms (hereinafter, also referred to as “nitrogen-containing compound (III)”), an amide group-containing compound, a urea compound, and a nitrogen-containing heterocyclic compound.

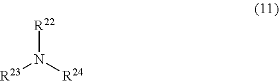

In the above formula (11), R²², R²³, and R²⁴ each independently represent a hydrogen atom, a substituted or unsubstituted alkyl group, a substituted or unsubstituted cycloalkyl group, a substituted or unsubstituted aryl group, or a substituted or unsubstituted aralkyl group.

Examples of the nitrogen-containing compound (I) include:
monoalkylamines such as n-hexylamine;
dialkylamines such as di-n-butylamine;
trialkylamines such as triethylamine; and
aromatic amines such as aniline.

Examples of the nitrogen-containing compound (II) include ethylenediamine and N,N,N′,N′-tetramethylethylenediamine.

Examples of the nitrogen-containing compound (III) include:
polyamine compounds such as polyethyleneimine and polyallylamine; and
polymers such as dimethylaminoethylacrylamide.

Examples of the amide group-containing compound include formamide, N-methylformamide, N,N-dimethylformamide, acetamide, N-methylacetamide, N,N-dimethylacetamide, propionamide, benzamide, pyrrolidone, and N-methylpyrrolidone.

Examples of the urea compound include urea, methylurea, 1,1-dimethylurea, 1,3-dimethylurea, 1,1,3,3-tetramethylurea, 1,3-diphenylurea, and tributylthiourea.

Examples of the nitrogen-containing heterocyclic compound include:
pyridines such as pyridine and 2-methylpyridine;
morpholines such as N-propylmorpholine and N-(undecylcarbonyloxyethyl) morpholine; and
pyrazines, and pyrazoles.

As the nitrogen-containing organic compound, a compound having an acid-dissociable group can also be used. Examples of such a nitrogen-containing organic compound having an acid-dissociable group include N-t-butoxycarbonylpiperidine, N-t-butoxycarbonylimidazole, N-t-butoxycarbonylbenzimidazole, N-t-butoxycarbonyl-2-phenylbenzimidazole, N-(t-butoxycarbonyl) di-n-octylamine, N-(t-butoxycarbonyl) diethanolamine, N-(t-butoxycarbonyl) dicyclohexylamine, N-(t-butoxycarbonyl) diphenylamine, N-t-butoxycarbonyl-4-hydroxypiperidine, N-t-butoxycarbonyl-4-acetoxypiperidine, and N-t-amyloxycarbonyl-4-hydroxypiperidine.

As the other acid diffusion controlling agent, a radiation-sensitive weak acid generator that generates a weak acid by exposure to light can be suitably used. The acid generated from the radiation-sensitive weak acid generator is a weak acid that does not induce dissociation of the acid-dissociable group under the condition of dissociating the acid-dissociable group in the resin B. In the present description, the “dissociation” of an acid-dissociable group refers to dissociating when post-exposure baking is performed at 110° C. for 60 seconds.

Examples of the radiation-sensitive weak acid generator include an onium salt compound that is decomposed by exposure to light to lose the acid diffusion controllability thereof. Examples of the onium salt compound include a sulfonium salt compound represented by the following formula (12-1) and an iodonium salt compound represented by the following formula (12-2).

J⁺E⁻ (12-1)

U⁺Q⁻ (12-2)

In the above formulas (12-1) and (12-2), J⁺ is a sulfonium cation, and U₊ is an iodonium cation.

Examples of the sulfonium cation represented by J⁺ include sulfonium cations represented by the above formulas (X-1) to (X-3), and examples of the iodonium cation represented by U⁺ include iodonium cations represented by the above formulas (X-4) to (X-5).

E⁻ and Q⁻ each independently are an anion represented by OH⁻, R^{α}—COO⁻, or R^{α}—SO₃⁻.

R^{α} is an alkyl group, an aryl group, or an aralkyl group. The hydrogen atom of the aromatic ring of the aryl group or the aralkyl group represented by R^{α} may be substituted with a hydroxy group, a fluorine atom-substituted or unsubstituted alkyl group having 1 to 12 carbon atoms, or an alkoxy group having 1 to 12 carbon atoms.

Examples of the radiation-sensitive weak acid generator include compounds represented by the following formulas.

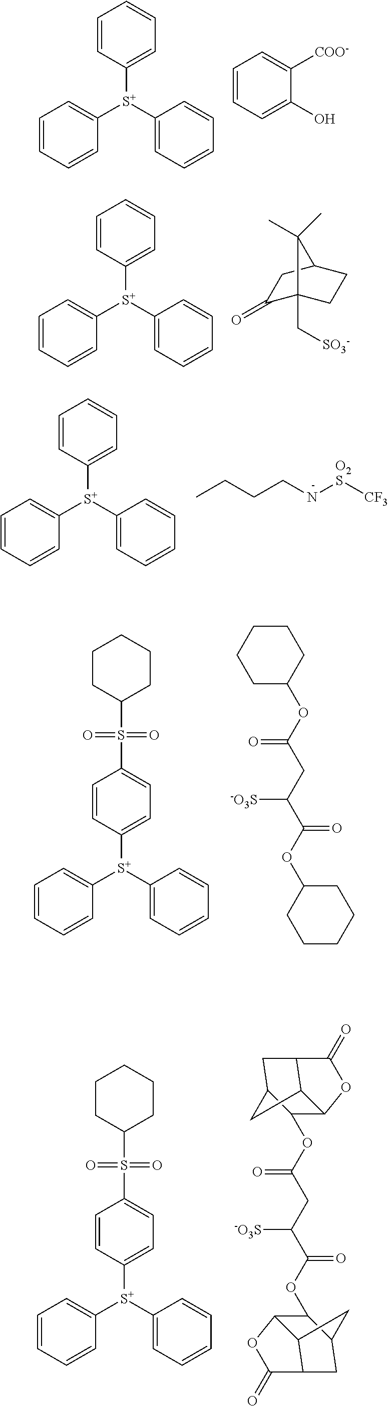

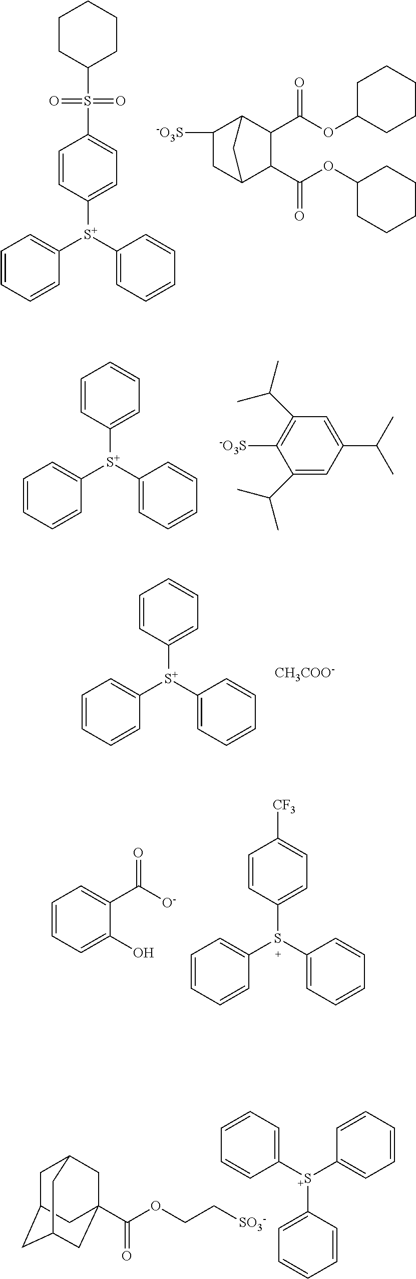

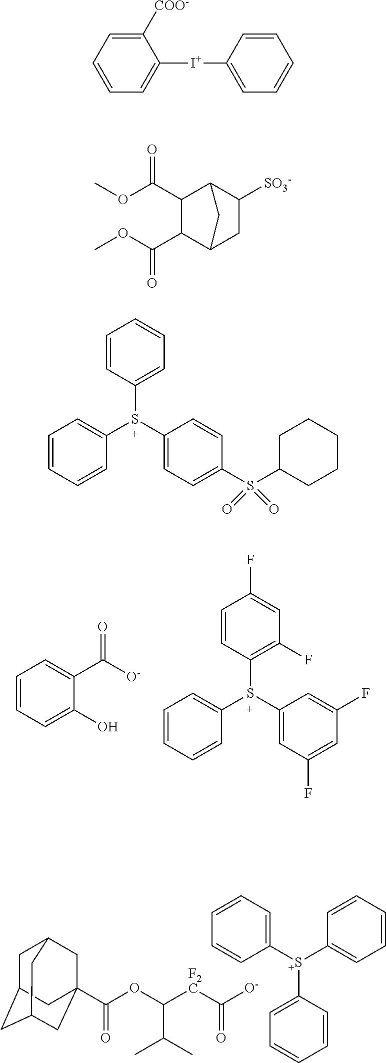

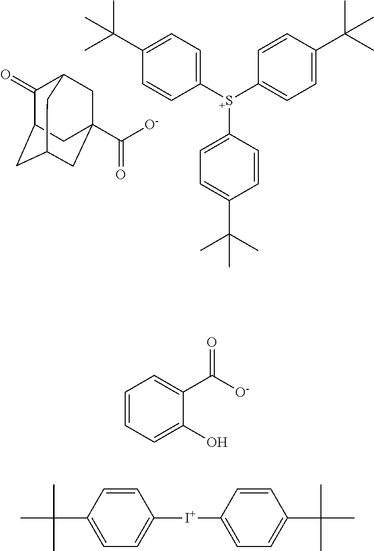

Among them, sulfonium salts are preferable as the radiation-sensitive weak acid generator, triarylsulfonium salts are more preferable, and triphenylsulfonium salicylate and triphenylsulfonium 10-camphorsulfonate are still more preferable.

The lower limit of the content of the other acid diffusion controlling agent is preferably 0.1 parts by mass, more preferably 0.5 parts by mass, still more preferably 1 part by mass, and particularly preferably 2 parts by mass per 100 parts by mass of the resin B. The upper limit of the content is preferably 20 parts by mass, more preferably 15 parts by mass, and still more preferably 10 parts by mass.

When the content of the other acid diffusion controlling agent is adjusted to within the above range, the lithographic performance of the radiation-sensitive resin composition can further be improved. The radiation-sensitive resin composition may contain one other acid diffusion controlling agent or two or more acid diffusion controlling agents.

### <Solvent>

The radiation-sensitive resin composition according to the present embodiment contains a solvent. The solvent is not particularly limited as long as it is a solvent capable of dissolving or dispersing at least a compound A, a base resin (a radiation-sensitive acid generating resin and at least one of the resins), a radiation-sensitive acid generator, and additives which are contained as desired.

Examples of the solvent include an alcohol-based solvent, an ether-based solvent, a ketone-based solvent, an amide-based solvent, an ester-based solvent, and a hydrocarbon-based solvent.

Examples of the alcohol-based solvent include:
monoalcohol-based solvents having 1 to 18 carbon atoms, such as iso-propanol, 4-methyl-2-pentanol, 3-methoxybutanol, n-hexanol, 2-ethylhexanol, furfuryl alcohol, cyclohexanol, 3,3,5-trimethylcyclohexanol, and diacetone alcohol;
polyhydric alcohol-based solvents having 2 to 18 carbon atoms, such as ethylene glycol, 1,2-propylene glycol, 2-methyl-2,4-pentanediol, 2,5-hexanediol, diethylene glycol, dipropylene glycol, triethylene glycol, and tripropylene glycol; and
partially etherized polyhydric alcohol-based solvents in which some of the hydroxy groups of the polyhydric alcohol-based solvent are etherized.

Examples of the ether-based solvent include:
dialkyl ether-based solvents, such as diethyl ether, dipropyl ether, and dibutyl ether;
cyclic ether-based solvents, such as tetrahydrofuran and tetrahydropyran;
aromatic ring-containing ether-based solvents, such as diphenyl ether and anisole (methyl phenyl ether); and
etherized polyhydric alcohol-based solvents in which the hydroxy groups of the polyhydric alcohol-based solvent are etherized.

Examples of the ketone-based solvent include chain ketone-based solvents, such as acetone, butanone, and methyl-iso-butyl ketone;
cyclic ketone-based solvents, such as cyclopentanone, cyclohexanone, and methylcyclohexanone; and
2,4-pentanedione, acetonylacetone, and acetophenone.

Examples of the amide-based solvent include cyclic amide-based solvents, such as N,N′-dimethylimidazolidinone and N-methylpyrrolidone; and
chain amide-based solvents, such as N-methylformamide, N,N-dimethylformamide, N,N-diethylformamide, acetamide, N-methylacetamide, N,N-dimethylacetamide, and N-methylpropionamide.

Examples of the ester-based solvent include:
monocarboxylic acid ester-based solvents, such as n-butyl acetate and ethyl lactate;
polyhydric alcohol partial ether acetate-based solvents, such as diethylene glycol mono-n-butyl ether acetate, propylene glycol monomethyl ether acetate, and dipropylene glycol monomethyl ether acetate;
lactone-based solvents, such as γ-butyrolactone and valerolactone;
carbonate-based solvents, such as diethyl carbonate, ethylene carbonate, and propylene carbonate; and
polyvalent carboxylic acid diester-based solvents, such as propylene glycol diacetate, methoxytriglycol acetate, diethyl oxalate, ethyl acetoacetate, ethyl lactate, and diethyl phthalate.

Examples of the hydrocarbon-based solvent include:
aliphatic hydrocarbon-based solvents, such as n-hexane, cyclohexane, and methylcyclohexane; and
aromatic hydrocarbon-based solvents, such as benzene, toluene, di-iso-propylbenzene, and n-amylnaphthalene.

Among them, ester-based solvents and ketone-based solvents are preferable, polyhydric alcohol partial ether acetate-based solvents, cyclic ketone-based solvents, and lactone-based solvents are more preferable, and propylene glycol monomethyl ether acetate, cyclohexanone, and γ-butyrolactone are still more preferable. The radiation-sensitive resin composition may contain one or two or more solvents.

### <Other Optional Components>

The radiation-sensitive resin composition may contain other optional components in addition to the components described above. Examples of the other optional components include a crosslinking agent, a localization enhancing agent, a surfactant, an alicyclic backbone-containing compound, and a sensitizer. Such other optional components may be used singly or two or more types thereof may be used in combination.

### <Method for Preparing Radiation-Sensitive Resin Composition>

The radiation-sensitive resin composition can be prepared, for example, by mixing a compound A, a base resin (at least one of a radiation-sensitive acid generating resin and a resin), a radiation-sensitive acid generator, and a solvent, and as necessary, other optional component at a prescribed ratio. The radiation-sensitive resin composition is preferably filtered through, for example, a filter having a pore size of approximately 0.05 μm to 0.2 μm after mixing. The solid concentration of the radiation-sensitive resin composition is usually from 0.1% by mass to 50% by mass, preferably from 0.5% by mass to 30% by mass, and more preferably from 1% by mass to 20% by mass.

### <Pattern Formation Method>

The pattern formation method according to the present disclosure comprises:
a step (1) of applying the radiation-sensitive resin composition directly or indirectly on a substrate to form a resist film (hereinafter also referred to as “resist film forming step”);
a step (2) of exposing the resist film to light (hereinafter also referred to as “exposure step”); and a step (3) of developing the exposed resist film with a developer (hereinafter also referred to as “development step”).

In accordance with this pattern formation method, a high-quality resist pattern can be formed because of the use of the radiation-sensitive resin composition superior in sensitivity and CDU performance in an exposure step. Each of the steps will be described below.

### [Resist Film Forming Step]

In this step (step (1)), a resist film is formed from the radiation-sensitive resin composition. Examples of the substrate on which the resist film is formed include conventionally known substrates such as a silicon wafer, silicon dioxide, and a wafer coated with aluminum. An organic or inorganic antireflective film disclosed in, for example, JP-B-6-12452 or JP-A-59-93448 may be formed on the substrate. Examples of a method for applying the composition include spin coating, cast coating, and roll coating. After the application, prebaking (PB) may be performed to volatilize the solvent in the coating film, as necessary. The PB temperature is usually 60° C. to 140° C., and preferably 80° C. to 120° C. The PB time is usually 5 seconds to 600 seconds, and preferably 10 seconds to 300 seconds. The thickness of the resist film to be formed is preferably 10 nm to 1,000 nm, and more preferably 10 nm to 500 nm.

In the case of performing immersion exposure, regardless of the presence or absence of a water repellent polymer additive such as the high fluorine-containing resin in the radiation-sensitive resin composition, a protective film for immersion insoluble in an immersion liquid may be provided on the formed resist film for the purpose of avoiding direct contact between the immersion liquid and the resist film. As the protective film for immersion, either a solvent-removable protective film that is to be removed by a solvent before the development step (see, for example, JP-A-2006-227632) or a developer-removable protective film that is to be removed simultaneously with the development in the development step (see, for example, WO 2005/069076 and WO 2006/035790) may be used. However, from the viewpoint of throughput, it is preferable to use a developer-removable protective film for immersion.

When the subsequent exposure step is performed with radiation having a wavelength of 50 nm or less, it is preferable to use a resin having the structural units (I) to (IV) and, as necessary, the structural unit (V) as the base resin in the composition.

### [Exposure Step]

In this step (the step (2)), the resist film formed in the resist film forming step, namely the step (1), is irradiated with radiation through a photomask (as the case may be, through an immersion medium such as water) to be exposed. Examples of the radiation to be used for the exposure include an electromagnetic wave including visible ray, ultraviolet ray, far ultraviolet ray, extreme ultraviolet ray (EUV), X ray, and γ ray; an electron beam; and a charged particle radiation such as α ray. Among them, far ultraviolet ray, electron beam, and EUV are preferable, ArF excimer laser light (wavelength: 193 nm), KrF excimer laser light (wavelength: 248 nm), electron beam, and EUV are more preferable, and an electron beam and EUV having a wavelength of 50 nm or less, which are positioned as next-generation exposure technology, are still more preferable.

When the exposure is performed by immersion exposure, examples of the immersion liquid to be used include water and a fluorine-based inert liquid. The immersion liquid is preferably a liquid that is transparent to an exposure wavelength and has a temperature coefficient of refractive index as small as possible to minimize the distortion of an optical image projected onto the film. Particularly, when an exposure light source is ArF excimer laser light (wavelength: 193 nm), water is preferably used from the viewpoint of availability and ease of handling in addition to the above-described viewpoints. When water is used, an additive that reduces the surface tension of water and increases the surface activity may be added in a small proportion. This additive is preferably one that does not dissolve the resist film on a wafer and has negligible influence on an optical coating at an under surface of a lens. The water to be used is preferably distilled water.

After the exposure, post exposure baking (PEB) is preferably carried out to promote the dissociation of the acid-dissociable group of the resin or the like due to the acid generated from the radiation-sensitive acid generator through the exposure in the exposed area of the resist film. As a result of the PEB, there is produced a difference in solubility in the developer between the exposed area and the unexposed area. The PEB temperature is usually 50° C. to 180° C., and preferably 80° C. to 130° C. The PEB time is usually 5 seconds to 600 seconds, and preferably 10 seconds to 300 seconds.

### [Development Step]

In this step (the step (3)), the resist film exposed in the exposure step, namely the step (2), is developed with a developer. Thus, a prescribed resist pattern can be formed. In a common procedure, after the development, the film is washed with a rinsing liquid such as water or alcohol and dried.

In the step (3), a negative-tone pattern can be formed by development with an organic solvent. In the step (3), a positive-tone pattern can be formed by development with an alkaline developer.

Examples of the developer to be used for the development include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, and 1,5-diazabicyclo-[4.3.0]-5-nonene. Among them, the aqueous TMAH solution is preferable, and a 2.38% by mass aqueous TMAH solution is more preferable.

In the case of organic solvent development, examples of the solvent include organic solvents such as hydrocarbon-based solvents, ether-based solvents, ester-based solvents, ketone-based solvents, and alcohol-based solvents, and solvents containing an organic solvent. Examples of the organic solvent include one or two or more solvents among the solvents listed as the solvent for the radiation-sensitive resin composition. Among them, ester-based solvents and ketone-based solvents are preferable. As the ester-based solvents, acetate-based solvents are preferable, and n-butyl acetate and amyl acetate are more preferable. As the ketone-based solvents, chain ketones are preferable, and 2-heptanone is more preferable. The content of the organic solvent in the developer is preferably 80% by mass or more, more preferably 90% by mass or more, still more preferably 95% by mass or more, and particularly preferably 99% by mass or more. Examples of the components other than the organic solvent in the developer include water and silicon oil. The radiation-sensitive resin composition of the present disclosure can be particularly suitably used when a step of forming a negative-tone pattern by development with an organic solvent is included.

Examples of a development method include a method in which a substrate is immersed in a bath filled with a developer for a certain period of time (dipping method), a method in which a developer is allowed to be present on a surface of a substrate due to surface tension and to stand for a certain period of time (puddle method), a method in which a developer is sprayed onto a surface of a substrate (spray method), and a method in which a developer is discharged onto a substrate that is rotated at a constant speed while a developer discharge nozzle is scanned at a constant speed (dynamic dispensing method).

### <Method for Manufacturing Substrate>

The method for manufacturing a substrate according to the present embodiment includes:
a step (4) of forming a pattern on a substrate using the pattern formed by the method described above as a mask.

In accordance with the method for manufacturing a substrate, a high-quality substrate can be efficiently formed because of the use of the pattern.

In the step (4), as a method of forming the pattern on the substrate using the pattern as a mask, a known method can be appropriately used.

### EXAMPLES

Hereinafter, the present invention will specifically be described with reference to synthesis examples, examples, and comparative examples, but is not limited to the following examples. Methods for measuring various physical property values are shown below.

### [Mw and Mn]

The Mw and the Mn of polymers were measured by gel permeation chromatography (GPC) using GPC columns manufactured by Tosoh Corporation (“G2000HXL”×2, “G3000HXL”×1, “G4000HXL”×1) under the following conditions.
Eluant: tetrahydrofuran (manufactured by Wako Pure Chemical Industries, Ltd.)
Flow rate: 1.0 mL/min
Sample concentration: 1.0% by mass
Amount of sample injected: 100 μL
Column temperature: 40° C.
Detector: differential refractometer
Standard substance: monodisperse polystyrene

### [¹³C-NMR Analysis]

¹³C-NMR analysis of polymers was performed using a nuclear magnetic resonance apparatus (“JNM-Delta 400” manufactured by JEOL Ltd.). Measurement was performed using deuterated chloroform as a measurement solvent.

### <Synthesis of Resin and High Fluorine-Containing Resin>

Monomers used for synthesis of resins and high fluorine-containing resins in Examples and Comparative Examples are shown below. In the following synthesis examples, unless otherwise specified, “parts by mass” means a value taken when the total mass of the monomers used is 100 parts by mass, and “mol %” means a value taken when the total number of moles of the monomers used is 100 mol %.

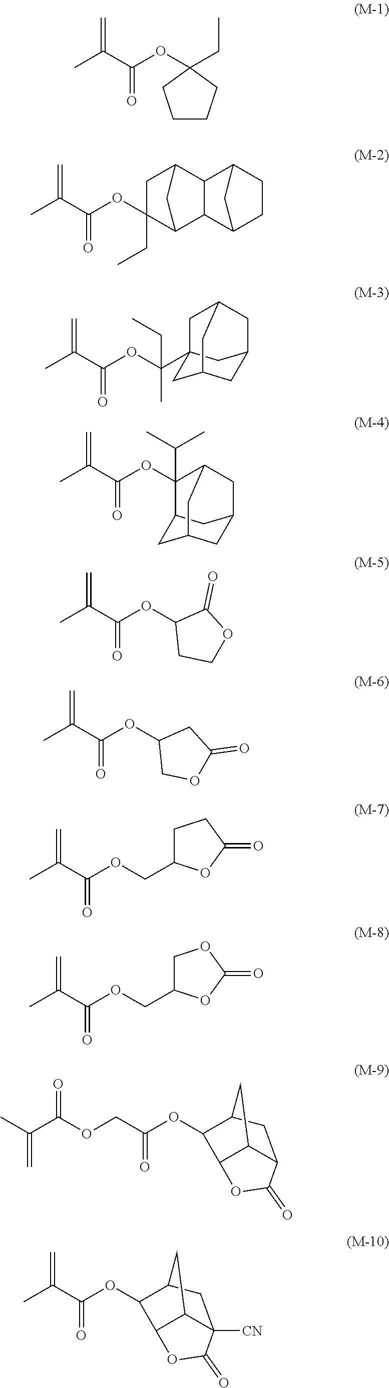

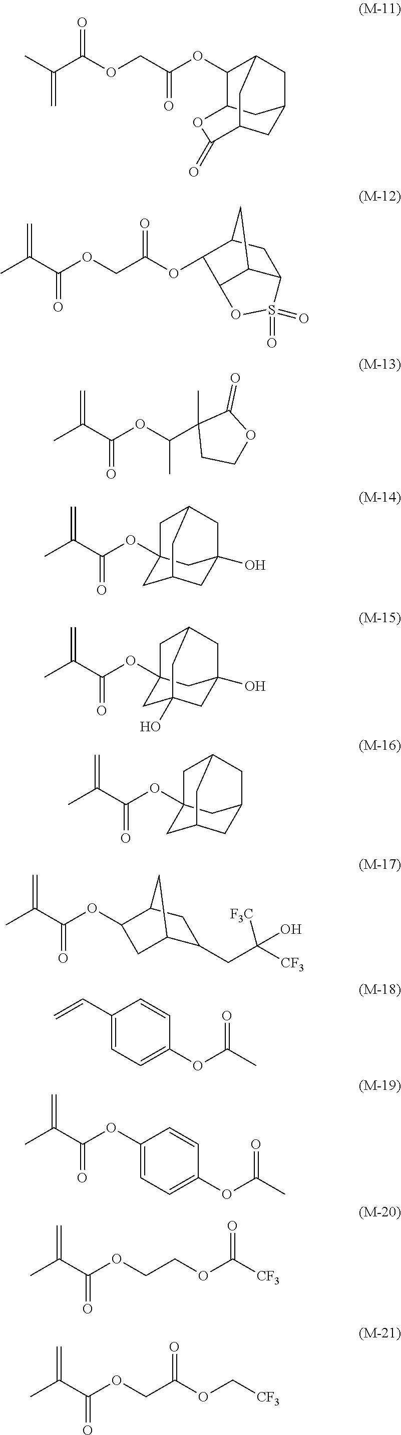

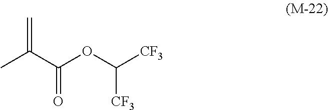

### Synthesis Example 1

### (Synthesis of Resin (A-1))

A monomer (M-1), a monomer (M-2), and a monomer (M-13) were dissolved at a molar ratio of 40/15/45 (mol %) in 2-butanone (200 parts by mass), and AIBN (azobisisobutyronitrile) (3 mol % based on 100 mol % in total of the monomers used) was added thereto as an initiator to prepare a monomer solution.

A reaction vessel was charged with 2-butanone (100 parts by mass), and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The polymerization solution cooled was poured into methanol (2,000 parts by mass), and a precipitated white powder was collected by filtration.

The white powder separated by filtration was washed with methanol twice, then separated by filtration, and dried at 50° C. for 24 hours to obtain a white powdery resin (A-1) (yield: 83%). The resin (A-1) had an Mw of 8,800 and an Mw/Mn of 1.50. As a result of ¹³C-NMR analysis, the content ratios of the structural units derived from (M-1), (M-2), and (M-13) were respectively 41.3 mol %, 13.8 mol %, and 44.9 mol %.

### Synthesis Examples 1 to 11

### (Synthesis of Resins (A-2) to (A-Li))

Resins (A-2) to (A-li) were synthesized in the same manner as in Synthesis Example 1 except that monomers of types and blending ratios shown in the following Table 1 were used. The content ratio (mol %), yield (%), and physical property values (Mw and Mw/Mn) of each of the structural units of the resulting resins are also shown in the following Table 1. In the following Table 1, “-” indicates that the corresponding monomer is not used (the same applies to the following Tables).

TABLE 1

| | | Monomer that gives | | | Monomer that gives | | | Monomer that gives | | | | |
|---|---|---|---|---|---|---|---|---|---|---|---|---|
| | | structural unit (I) | | | structural unit (II) | | | structural unit (III) | | | | |
| | | | | Content | | | Content | | | Content | | |
| | | | | ratio of | | | ratio of | | | ratio of | | |
| | | | Blending | structural | | Blending | structural | | Blending | structural | | |
| | Polymer | | ratio | unit | | ratio | unit | | ratio | unit | | |
| | [A] | Type | (mol %) | (mol %) | Type | (mol %) | (mol %) | Type | (mol %) | (mol %) | Mw | Mw/Mn |
| Synthesis | A-1 | M-1 | 40 | 41.3 | M-13 | 45 | 44.9 | — | — | — | 8800 | 1.50 |
| Example 1 | | M-2 | 15 | 13.8 | | | | | | | | |
| Synthesis | A-2 | M-1 | 30 | 31.4 | M-6 | 60 | 60.6 | — | — | — | 9000 | 1.44 |
| Example 2 | | M-2 | 10 | 8.0 | | | | | | | | |
| Synthesis | A-3 | M-1 | 30 | 31.9 | M-5 | 60 | 59.2 | — | — | — | 8600 | 1.51 |
| Example 3 | | M-3 | 10 | 8.9 | | | | | | | | |
| Synthesis | A-4 | M-1 | 35 | 34.8 | M-12 | 45 | 46.4 | — | — | — | 7700 | 1.56 |
| Example 4 | | M-3 | 20 | 18.8 | | | | | | | | |
| Synthesis | A-5 | M-1 | 40 | 41.1 | M-10 | 45 | 46.8 | — | — | — | 7900 | 1.44 |
| Example 5 | | M-4 | 15 | 12.1 | | | | | | | | |
| Synthesis | A-6 | M-1 | 40 | 40.7 | M-11 | 45 | 46.1 | — | — | — | 8100 | 1.45 |
| Example 6 | | M-4 | 15 | 13.2 | | | | | | | | |
| Synthesis | A-7 | M-1 | 40 | 42.4 | M-10 | 45 | 39.5 | M-14 | 15 | 18.1 | 7800 | 1.59 |
| Example 7 | | | | | | | | | | | | |
| Synthesis | A-8 | M-1 | 40 | 40.2 | M-7 | 40 | 41.1 | M-15 | 20 | 18.7 | 8500 | 1.61 |
| Example 8 | | | | | | | | | | | | |
| Synthesis | A-9 | M-1 | 50 | 51.0 | M-8 | 50 | 49.0 | — | — | — | 7800 | 1.55 |
| Example 9 | | | | | | | | | | | | |
| Synthesis | A-10 | M-1 | 40 | 41.3 | M-9 | 60 | 58.7 | — | — | — | 8200 | 1.55 |
| Example 10 | | | | | | | | | | | | |
| Synthesis | A-11 | M-1 | 40 | 42.8 | M-6 | 60 | 57.2 | — | — | — | 8000 | 1.43 |
| Example 11 | | | | | | | | | | | | |

### Synthesis Example 12

### (Synthesis of Resin (A-12))

Monomers (M-1) and (M-18) were dissolved at a molar ratio of 50/50 (mol %) in 1-methoxy-2 propanol (200 parts by mass), and AIBN (5 mol %) was added thereto as an initiator to prepare a monomer solution.

A reaction vessel was charged with 1-methoxy-2-propanol (100 parts by mass), and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The cooled polymerization solution was poured into hexane (2,000 parts by mass), and a precipitated white powder was collected by filtration.

The white powder separated by filtration was washed with hexane twice, then separated by filtration, and dissolved in 1-methoxy-2-propanol (300 parts by mass). Next, methanol (500 parts by mass), triethylamine (50 parts by mass) and ultrapure water (10 parts by mass) were added, and a hydrolysis reaction was performed at 70° C. for 6 hours with stirring.

After the completion of the reaction, the remaining solvent was distilled off. The resulting solid was dissolved in acetone (100 parts by mass), and the solution was added dropwise to water (500 parts by mass) to solidify a resin. The resulting solid was separated by filtration, and dried at 50° C. for 13 hours to obtain a white powdery resin (A-12) (yield: 79%). The resin (A-12) had an Mw of 5,200 and an Mw/Mn of 1.60. As a result of ¹³C-NMR analysis, the content ratios of the structural units derived from (M-1) and (M-18) were respectively 51.3 mol % and 48.7 mol %.

### [Synthesis Examples 13 to 15]

### (Synthesis of Resins (A-13) to (A-15))

Resins (A-13) to (A-15) were synthesized in the same manner as in Synthesis Example 12 except that monomers of types and blending ratios shown in the following Table 2 were used. The content ratio (mol %), yield (%), and physical property values (Mw and Mw/Mn) of each of the structural units of the resulting resins are also shown in the following Table 2.

TABLE 2

| | | Monomer that gives | | | Monomer that gives | | | Monomer that gives | | | | |
|---|---|---|---|---|---|---|---|---|---|---|---|---|
| | | structural unit (I) | | | structural unit (II) | | | structural unit (III) | | | | |
| | | | | Content | | | Content | | | Content | | |
| | | | | ratio of | | | ratio of | | | ratio of | | |
| | | | Blending | structural | | Blending | structural | | Blending | structural | | |
| | Polymer | | ratio | unit | | ratio | unit | | ratio | unit | | |
| | [A] | Type | (mol %) | (mol %) | Type | (mol %) | (mol %) | Type | (mol %) | (mol %) | Mw | Mw/Mn |
| Synthesis | A-12 | M-1 | 50 | 51.3 | — | — | — | M-18 | 50 | 48.7 | 5200 | 1.60 |
| Example 12 | | | | | | | | | | | | |
| Synthesis | A-13 | M-3 | 50 | 47.9 | M-14 | 10 | 10.3 | M-19 | 40 | 41.8 | 5500 | 1.53 |
| Example 13 | | | | | | | | | | | | |
| Synthesis | A-14 | M-2 | 50 | 48.1 | M-17 | 20 | 21.3 | M-18 | 30 | 30.6 | 5100 | 1.59 |
| Example 14 | | | | | | | | | | | | |
| Synthesis | A-15 | M-1 | 55 | 55.7 | M-17 | 15 | 15.1 | M-19 | 30 | 29.2 | 6100 | 1.50 |
| Example 15 | | | | | | | | | | | | |

### Synthesis Example 16

### (Synthesis of High Fluorine-Containing Resin (E-1))

Monomers (M-1) and (M-20) were dissolved at a molar ratio of 20/80 (mol %) in 2-butanone (200 parts by mass), and AIBN (4 mol %) was added thereto as an initiator to prepare a monomer solution.

A reaction vessel was charged with 2-butanone (100 parts by mass), and the reaction vessel was purged with nitrogen for 30 minutes. Then, the temperature inside the reaction vessel was adjusted to 80° C., and the monomer solution was added dropwise thereto over 3 hours with stirring. A polymerization reaction was performed for 6 hours with the start of the dropwise addition regarded as the start time of the polymerization reaction. After the completion of the polymerization reaction, the polymerization solution was cooled with water to 30° C. or lower. The solvent was replaced with acetonitrile (400 parts by mass). Hexane (100 parts by mass) was then added, followed by stirring, and an acetonitrile layer was collected. The operation was repeated three times.

By replacing the solvent with propylene glycol monomethyl ether acetate, a solution of a high fluorine-containing resin (E-1) was obtained (yield: 69%). The high fluorine-containing resin (E-1) had an Mw of 6,000 and an Mw/Mn of 1.62. As a result of ¹³C-NMR analysis, the content ratios of the structural units derived from (M-1) and (M-20) were respectively 19.9 mol % and 80.1 mol %.

### Synthesis Examples 17 to 20

### (Synthesis of High Fluorine-Containing Resins (E-2) to (E-5))

High fluorine-containing resins (E-2) to (E-5) were synthesized in the same manner as in Synthesis Example 16 except that monomers of types and blending ratios shown in the following Table 3 were used. The content ratio (mol %), yield (%), and physical property values (Mw and Mw/Mn) of each of the structural units of the resulting high fluorine-containing resins are also shown in the following Table 3.

TABLE 3

| | | Monomer that gives | | | Monomer that gives | | | Monomer that gives | | | Monomer that gives other | | | | |
|---|---|---|---|---|---|---|---|---|---|---|---|---|---|---|---|
| | | structural unit (F) | | | structural unit (I) | | | structural unit (II) | | | structural unit | | | | |
| | | | | Content | | | Content | | | Content | | | Content | | |
| | | | | ratio of | | | ratio of | | | ratio of | | | ratio of | | |
| | | | Blend- | struc- | | Blend- | struc- | | Blend- | struc- | | Blend- | struc- | | |
| | | | ing | tural | | ing | tural | | ing | tural | | ing | tural | | |
| | Polymer | | ratio | unit | | ratio | unit | | ratio | unit | | ratio | unit | | |
| | [E] | Type | (mol %) | (mol %) | Type | (mol %) | (mol %) | Type | (mol %) | (mol %) | Type | (mol %) | (mol %) | Mw | Mw/Mn |
| Synthesis | E-1 | M-20 | 80 | 80.1 | M-1 | 20 | 19.9 | — | — | — | — | — | — | 6000 | 1.62 |
| Example 16 | | | | | | | | | | | | | | | |
| Synthesis | E-2 | M-21 | 80 | 81.9 | M-1 | 20 | 18.1 | — | — | — | — | — | — | 7200 | 1.77 |
| Example 17 | | | | | | | | | | | | | | | |
| Synthesis | E-3 | M-22 | 60 | 62.3 | — | — | — | — | — | — | M-16 | 40 | 38.7 | 6300 | 1.82 |
| Example 18 | | | | | | | | | | | | | | | |
| Synthesis | E-4 | M-22 | 70 | 68.7 | — | — | — | M-14 | 30 | 31.3 | — | — | — | 6500 | 1.81 |
| Example 19 | | | | | | | | | | | | | | | |
| Synthesis | E-5 | M-20 | 60 | 59.2 | M-2 | 10 | 10.3 | M-17 | 30 | 30.5 | — | — | — | 6100 | 1.86 |
| Example 20 | | | | | | | | | | | | | | | |

### <Synthesis of Acid Diffusion Controlling Agent C>

### Synthesis Example 21

### (Synthesis of Compound (C-1))

A compound (C-1) was synthesized in accordance with the following synthesis scheme.

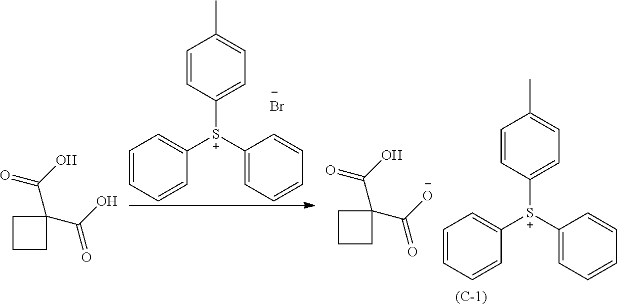

A reaction vessel was charged with 20.0 mmol of 1,1-cyclobutanedicarboxylic acid, 20.0 mmol of lithium hydroxide, and 20.0 mmol of diphenyl(p-tolyl)sulfonium bromide, and a mixed liquid of water and dichloromethane (1:3 (mass ratio)) was added thereto, forming a 0.5 M solution. The solution was vigorously stirred at room temperature for 3 hours. Thereafter, dichloromethane was added thereto, followed by extraction, and then the organic layer was separated. After the organic layer obtained was dried over sodium sulfate, the solvent was distilled off, and the residue was purified by recrystallization, affording a compound (C-1) represented by the above formula (C-1) in a good yield.

### Synthesis Examples 22 to 29

### (Synthesis of Compounds (C-2) to (C-9))

Onium salts represented by the following formulas (C-2) to (C-9) were synthesized in the same manner as in Synthesis Example 21 except that the raw materials and the precursor were appropriately changed.

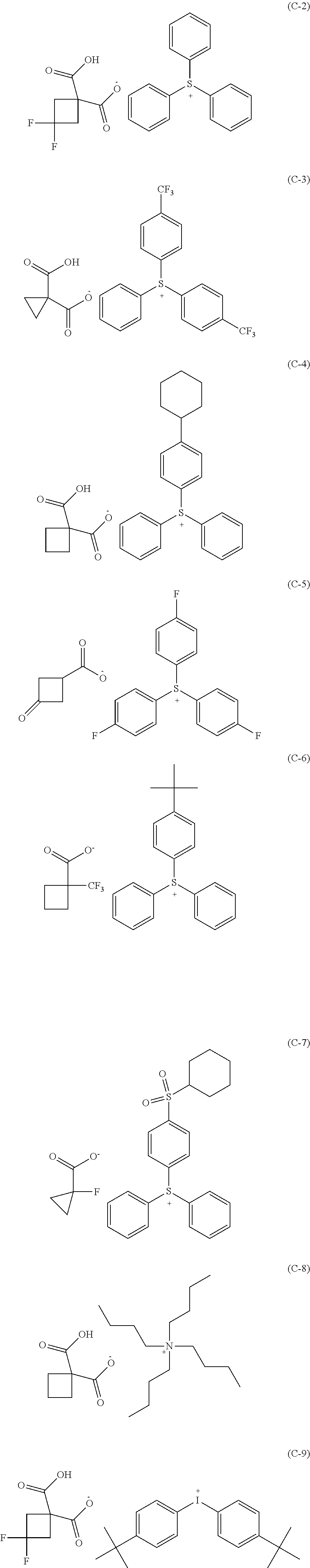

### Synthesis Example 30

### (Synthesis of Compound (C-10))

A compound (C-10) was synthesized in accordance with the following synthesis scheme.

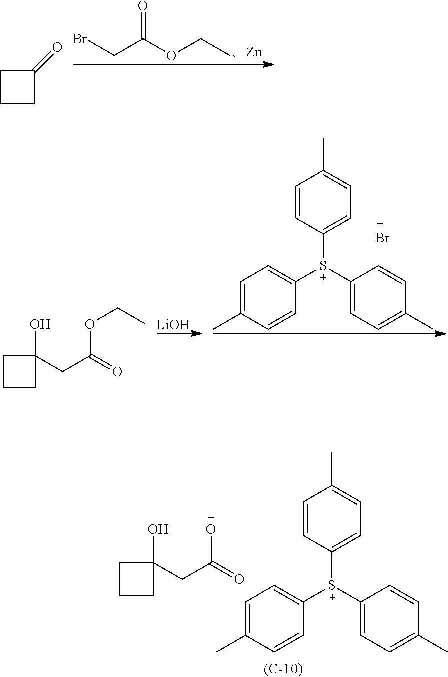

A reaction vessel was charged with 20.0 mmol of ethyl bromoacetate, 25.0 mmol of zinc powder, 2.00 mmol of chlorotrimethylsilane, and 50 g of tetrahydrofuran, and the mixture was stirred at room temperature for 1 hour. Thereafter, 20.0 mmol of cyclobutanone was added to the reaction solution, and the resulting mixture was further stirred at room temperature for 8 hours. Thereafter, a saturated aqueous solution of ammonium chloride was added to the reaction solution to terminate the reaction, and ethyl acetate was then added thereto to perform extraction, thereby separating an organic layer. The resulting organic layer was washed with a saturated aqueous solution of sodium chloride and then with water. After the organic layer obtained was dried over sodium sulfate, the solvent was distilled off, and the residue was purified by column chromatography, affording an alcohol form in good yield.

A mixed liquid of ethanol and water (1:1 (mass ratio)) was added to the alcohol form to form a 1 M solution. Then, 20.0 mmol of lithium hydroxide was added, and the resulting mixture was reacted at 50° C. for 2 hours. The mixture was extracted with acetonitrile, and the solvent was distilled off, affording a lithium salt derivative. To the lithium salt derivative was added 20.0 mmol of tri-p-tolylsulfonium bromide, and a mixed liquid of water and dichloromethane (1:3 (mass ratio)) was added thereto. The solution was vigorously stirred at room temperature for 3 hours. Thereafter, dichloromethane was added thereto, followed by extraction, and then the organic layer was separated. After the organic layer obtained was dried over sodium sulfate, the solvent was distilled off, and the residue was purified by recrystallization, affording a compound (C-10) represented by the above formula (C-10) in a good yield.

### Synthesis Examples 31 to 33

### (Synthesis of Compounds (C-11) to (C-13))

Onium salts represented by the following formulas (C-11) to (C-13) were synthesized in the same manner as in Synthesis Example 30 except that the raw materials and the precursor were appropriately changed.

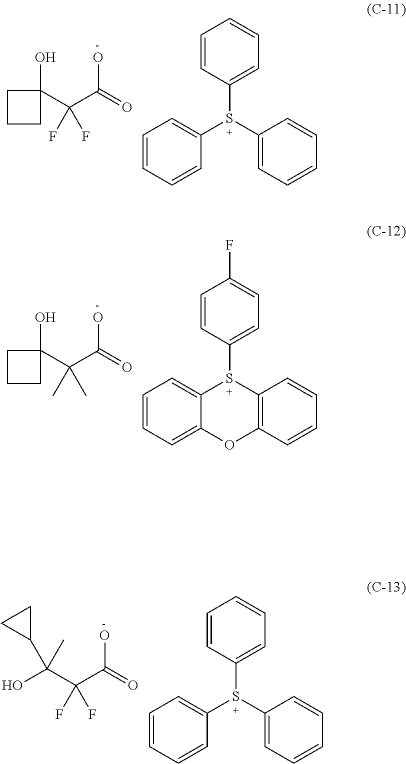

### Synthesis Example 34

### (Synthesis of Compound (C-14))

A compound (C-14) was synthesized in accordance with the following synthesis scheme.

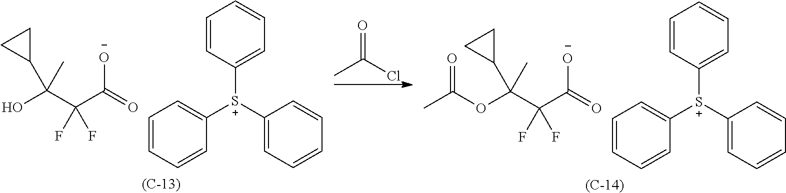

A reaction vessel was charged with 20.0 mmol of the compound (C-13), 25.0 mmol of acetyl chloride, 25.0 mmol of triethylamine, and 50 g of dichloromethane, and the mixture was stirred at room temperature for 10 hours. Thereafter, a saturated aqueous solution of ammonium chloride was added to the reaction solution to terminate the reaction, and dichloromethane was then added thereto to perform extraction, thereby separating an organic layer. The resulting organic layer was washed with a saturated aqueous solution of sodium chloride and then with water. After the organic layer obtained was dried over sodium sulfate, the solvent was distilled off, and the residue was purified by recrystallization, affording a compound (C-14) represented by the above formula (C-14) in a good yield.

### Synthesis Example 35

### (Synthesis of Compound (C-15))

A compound (C-15) was synthesized in accordance with the following synthesis scheme.

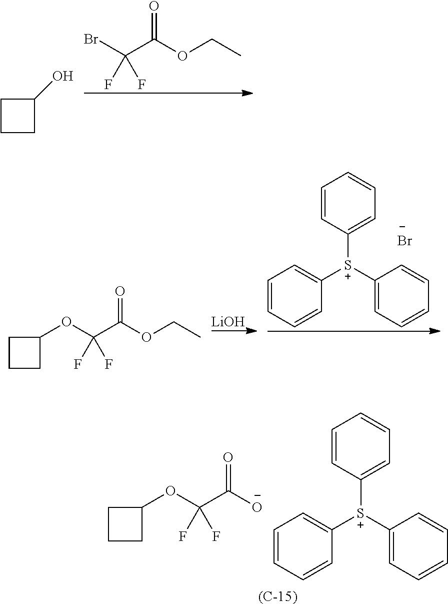

A reaction vessel was charged with 20.0 mmol of ethyl bromodifluoroacetate, 25.0 mmol of cyclobutanol, 30.0 mmol of 1,8-diazabicyclo[5,4,0]-7-undecene, and 50 g of dimethylformamide, and the mixture was stirred at 50° C. for 4 hours. Thereafter, a saturated aqueous solution of ammonium chloride was added to the reaction solution to terminate the reaction, and ethyl acetate was then added thereto to perform extraction, thereby separating an organic layer. The resulting organic layer was washed with a saturated aqueous solution of sodium chloride and then with water. After the organic layer obtained was dried over sodium sulfate, the solvent was distilled off, and the residue was purified by column chromatography, affording an ester form in good yield.

A mixed liquid of ethanol and water (1:1 (mass ratio)) was added to the ester form to form a 1 M solution. Then, 20.0 mmol of lithium hydroxide was added, and the resulting mixture was reacted at room temperature for 7 hours. The mixture was extracted with acetonitrile, and the solvent was distilled off, affording a lithium salt derivative. To the lithium salt derivative was added 20.0 mmol of triphenylsulfonium bromide, and a mixed liquid of water and dichloromethane (1:3 (mass ratio)) was added thereto. The solution was vigorously stirred at room temperature for 3 hours. Thereafter, dichloromethane was added thereto, followed by extraction, and then the organic layer was separated. After the organic layer obtained was dried over sodium sulfate, the solvent was distilled off, and the residue was purified by recrystallization, affording a compound (C-15) represented by the above formula (C-15) in a good yield.

### Synthesis Example 36

### (Synthesis of Compound (C-16))

A compound (C-16) was synthesized in accordance with the following synthesis scheme.

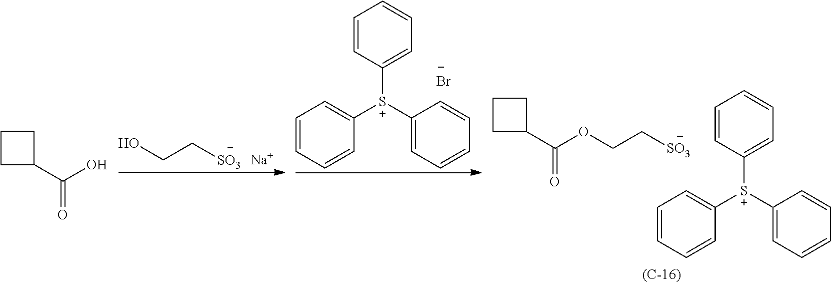

A reaction vessel was charged with 20.0 mmol of cyclobutanecarboxylic acid, 20.0 mmol of sodium isethionate, 30.0 mmol of 1-(3-dimethylaminopropyl)-3-ethylcarbodiimide hydrochloride, and 50 g of chloroform, and the mixture was stirred at 50° C. for 8 hours. Thereafter, water was added to the reaction solution to dilute the solution. Then, the resulting mixture was extracted with acetonitrile, and the solvent was distilled off, affording a sodium salt derivative. To the sodium salt derivative was added 20.0 mmol of triphenylsulfonium bromide, and a mixed liquid of water and dichloromethane (1:3 (mass ratio)) was added thereto. The solution was vigorously stirred at room temperature for 3 hours. Thereafter, dichloromethane was added thereto, followed by extraction, and then the organic layer was separated. After the organic layer obtained was dried over sodium sulfate, the solvent was distilled off, and the residue was purified by recrystallization, affording a compound (C-16) represented by the above formula (C-16) in a good yield.

### Synthesis Examples 37 to 39

### (Synthesis of Compounds (C-17) to (C-19))

Onium salts represented by the following formulas (C-17) to (C-19) were synthesized in the same manner as in Synthesis Example 34 except that the raw materials and the precursor were appropriately changed.

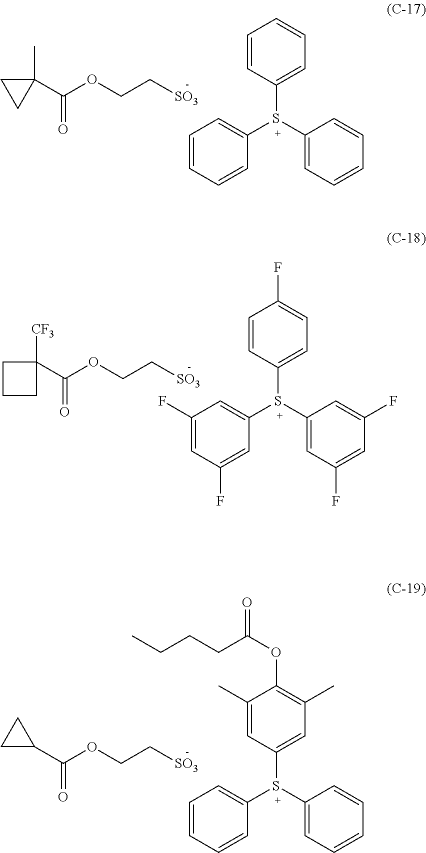

### Synthesis Example 40

### (Synthesis of Compound (C-20))

A compound (C-20) was synthesized in accordance with the following synthesis scheme.

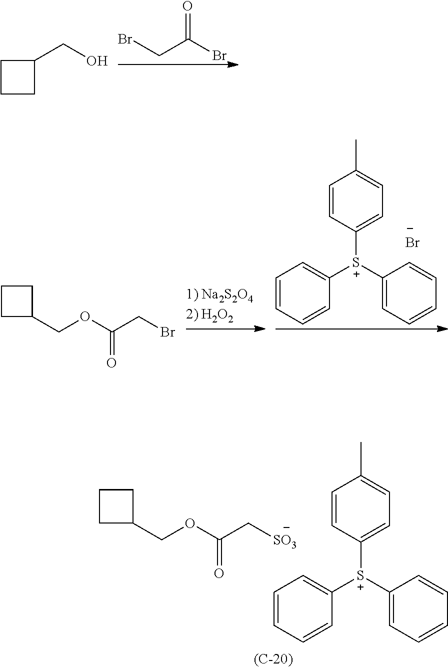

A reaction vessel was charged with 20.0 mmol of cyclobutane methanol, 20.0 mmol of bromoacetyl bromide, 30.0 mmol of triethylamine, and 50 g of tetrahydrofuran, and the mixture was stirred at room temperature for 4 hours. Thereafter, a saturated aqueous solution of ammonium chloride was added to the reaction solution to terminate the reaction, and ethyl acetate was then added thereto to perform extraction, thereby separating an organic layer. The resulting organic layer was washed with a saturated aqueous solution of sodium chloride and then with water. After the organic layer obtained was dried over sodium sulfate, the solvent was distilled off, and the residue was purified by column chromatography, affording a bromo body in a good yield.

A mixed liquid of acetonitrile and water (1:1 (mass ratio)) was added to the bromo body to form a 1 M solution. Then, 40.0 mmol of sodium dithionite and 60.0 mmol of sodium hydrogen carbonate were added, and the resulting mixture was reacted at 70° C. for 5 hours. After extraction with acetonitrile and subsequent distillation of the solvent, a mixed liquid of acetonitrile and water (3:1 (mass ratio)) was added to form a 0.5 M solution. 60.0 mmol of hydrogen peroxide water and 2.00 mmol of sodium tungstate were added, and the mixture was heated and stirred at 50° C. for 12 hours. The mixture was extracted with acetonitrile, and the solvent was distilled off, affording a sodium sulfonate salt compound. To the sodium sulfonate salt compound was added 20.0 mmol of diphenyl(p-tolyl)sulfonium bromide, and a mixed liquid of water and dichloromethane (1:3 (mass ratio)) was added thereto, forming a 0.5 M solution. The solution was vigorously stirred at room temperature for 3 hours. Thereafter, dichloromethane was added thereto, followed by extraction, and then the organic layer was separated. After the organic layer obtained was dried over sodium sulfate, the solvent was distilled off, and the residue was purified by recrystallization, affording a compound (C-20) represented by the above formula (C-20) in a good yield.

### Synthesis Examples 41 to 43

### (Synthesis of Compounds (C-21) to (C-23))

Onium salts represented by the following formulas (C-21) to (C-23) were synthesized in the same manner as in Synthesis Example 38 except that the raw materials and the precursor were appropriately changed.

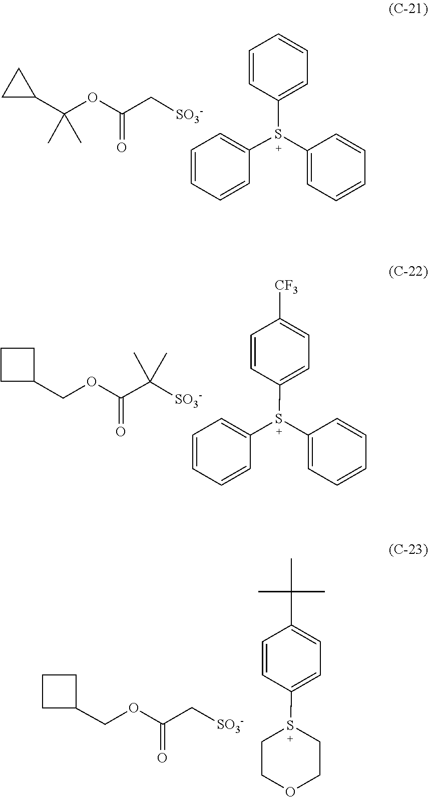

[Onium Salts Other than Compounds (C-1) to (C-21)]

cc-1 to cc-10: Compounds represented by the following formulas (cc-1) to (cc-10) (Hereinafter, the compounds represented by the formulas (cc-1) to (cc-10) may be described as “compound (cc-1)” to “compound (cc-10)”, respectively.)

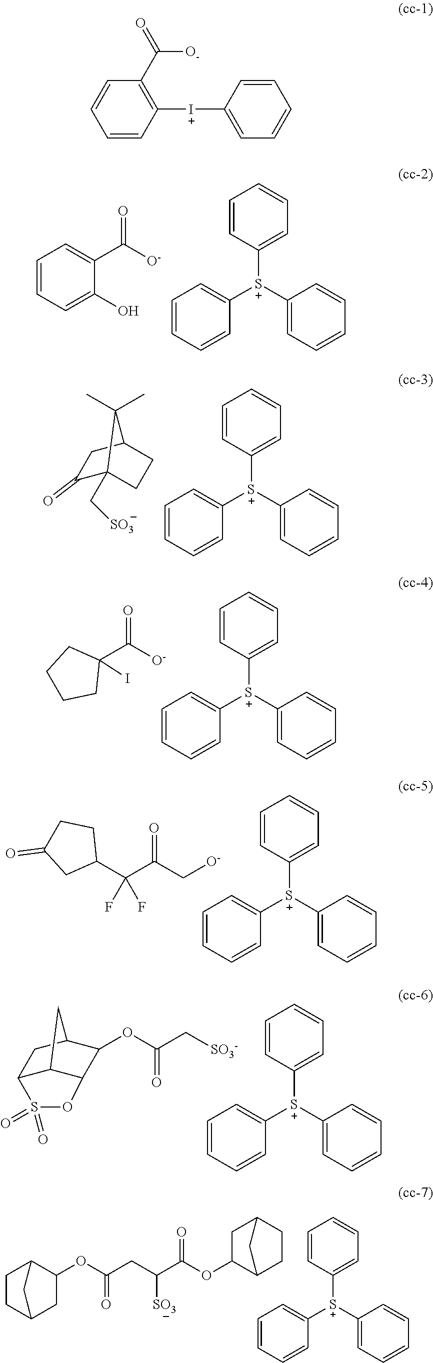

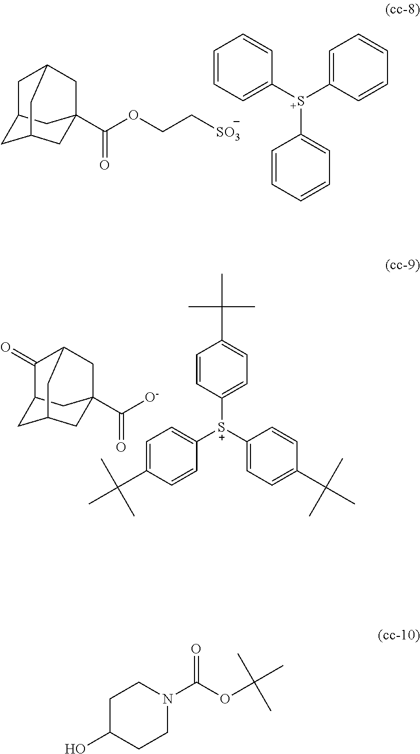

### [Radiation-Sensitive Acid Generator [B]]

B-1 to B-8: Compounds represented by the following formulas (B-1) to (B-8) (Hereinafter, the compounds represented by the formulas (B-1) to (B-8) may be described as “compound (B-1)” to “compound (B-8)”, respectively.)

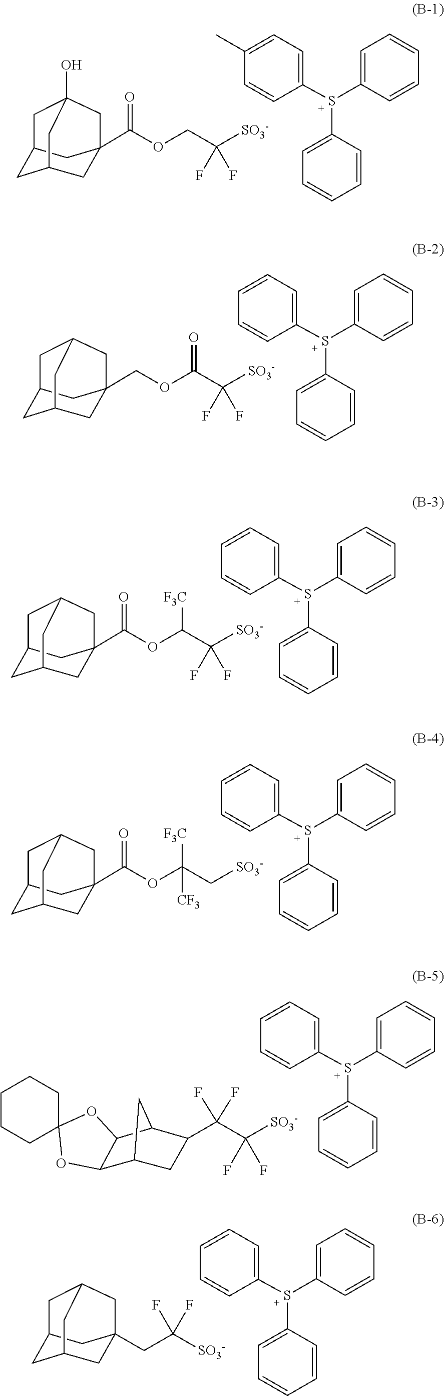

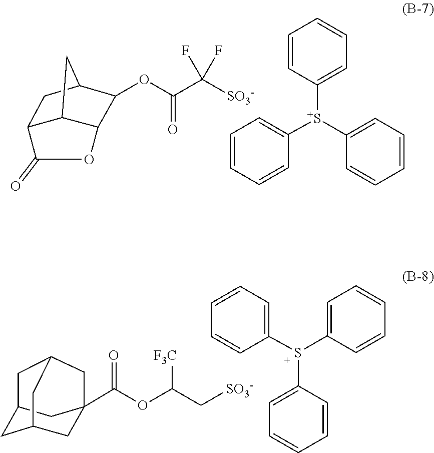

### [Solvent [D]]

D-1: Propylene glycol monomethyl ether acetate
D-2: Propylene glycol monomethyl ether
D-3: y-Butyrolactone
D-4: Ethyl lactate

### [Preparation of Negative Radiation-Sensitive Resin Composition for ArF Exposure]

### Example 1

100 parts by mass of (A-1) as the resin [A], 10.0 parts by mass of (B-1) as the radiation-sensitive acid generator [B], 5.0 parts by mass of (C-1) as the acid diffusion controlling agent [C], 3.0 parts by mass (solid content) of (E-1) as the high fluorine-containing resin [E], and 3,230 parts by mass of a mixed solvent of (D-1)/(D-2)/(D-3) as the solvent [D] were mixed, and the mixture was filtered through a membrane filter having a pore size of 0.2 μm to prepare a radiation-sensitive resin composition (J-1) C

### Examples 2 to 53 and Comparative Examples 1 to 10

Radiation-sensitive resin compositions (J-2) to (J-53) and (CJ-1) to (CJ-10) were prepared in the same manner as in Example 1 except that the components of the types and contents shown in the following Table 4 were used.

TABLE 4

| | | | | | | Acid diffusion | | | | | |
|---|---|---|---|---|---|---|---|---|---|---|---|
| | | Polymer [A] | | Acid generator [B] | | controlling agent [C] | | Polymer [E] | | Organic solvent [D] | |
| | Radiation- | | Content | | Content | | Content | | Content | | Content |
| | sensitive resin | | (parts | | (parts | | (parts | | (parts | | (parts |
| | composition | Type | by mass) | Type | by mass) | Type | by mass) | Type | by mass) | Type | by mass) |
| Example 1 | J-1 | A-1 | 100 | B-1 | 10.0 | C-1 | 5.0 | E-1 | 3.0 | D-1/D-2/D-3 | 2240/960/30 |
| Example 2 | J-2 | A-1 | 100 | B-1 | 10.0 | C-2 | 5.0 | E-1 | 3.0 | D-1/D-2/D-3 | 2240/960/30 |
| Example 3 | J-3 | A-1 | 100 | B-1 | 10.0 | C-3 | 5.0 | E-1 | 3.0 | D-1/D-2/D-3 | 2240/960/30 |
| Example 4 | J-4 | A-1 | 100 | B-1 | 10.0 | C-4 | 5.0 | E-1 | 3.0 | D-1/D-2/D-3 | 2240/960/30 |
| Example 5 | J-5 | A-1 | 100 | B-1 | 10.0 | C-5 | 5.0 | E-1 | 3.0 | D-1/D-2/D-3 | 2240/960/30 |
| Example 6 | J-6 | A-1 | 100 | B-1 | 10.0 | C-6 | 5.0 | E-1 | 3.0 | D-1/D-2/D-3 | 2240/960/30 |
| Example 7 | J-7 | A-1 | 100 | B-1 | 10.0 | C-7 | 5.0 | E-1 | 3.0 | D-1/D-2/D-3 | 2240/960/30 |
| Example 8 | J-8 | A-1 | 100 | B-1 | 10.0 | C-8 | 5.0 | E-1 | 3.0 | D-1/D-2/D-3 | 2240/960/30 |
| Example 9 | J-9 | A-1 | 100 | B-1 | 10.0 | C-9 | 5.0 | E-1 | 3.0 | D-1/D-2/D-3 | 2240/960/30 |
| Example 10 | J-10 | A-1 | 100 | B-1 | 10.0 | C-10 | 5.0 | E-1 | 3.0 | D-1/D-2/D-3 | 2240/960/30 |
| Example 11 | J-11 | A-1 | 100 | B-1 | 10.0 | C-11 | 5.0 | E-1 | 3.0 | D-1/D-2/D-3 | 2240/960/30 |
| Example 12 | J-12 | A-1 | 100 | B-1 | 10.0 | C-12 | 5.0 | E-1 | 3.0 | D-1/D-2/D-3 | 2240/960/30 |
| Example 13 | J-13 | A-1 | 100 | B-1 | 10.0 | C-13 | 5.0 | E-1 | 3.0 | D-1/D-2/D-3 | 2240/960/30 |
| Example 14 | J-14 | A-1 | 100 | B-1 | 10.0 | C-14 | 5.0 | E-1 | 3.0 | D-1/D-2/D-3 | 2240/960/30 |
| Example 15 | J-15 | A-1 | 100 | B-1 | 10.0 | C-15 | 5.0 | E-1 | 3.0 | D-1/D-2/D-3 | 2240/960/30 |
| Example 16 | J-16 | A-1 | 100 | B-1 | 10.0 | C-16 | 5.0 | E-1 | 3.0 | D-1/D-2/D-3 | 2240/960/30 |
| Example 17 | J-17 | A-1 | 100 | B-1 | 10.0 | C-17 | 5.0 | E-1 | 3.0 | D-1/D-2/D-3 | 2240/960/30 |
| Example 18 | J-18 | A-1 | 100 | B-1 | 10.0 | C-18 | 5.0 | E-1 | 3.0 | D-1/D-2/D-3 | 2240/960/30 |
| Example 19 | J-19 | A-1 | 100 | B-1 | 10.0 | C-19 | 5.0 | E-1 | 3.0 | D-1/D-2/D-3 | 2240/960/30 |
| Example 20 | J-20 | A-1 | 100 | B-1 | 10.0 | C-20 | 5.0 | E-1 | 3.0 | D-1/D-2/D-3 | 2240/960/30 |
| Example 21 | J-21 | A-1 | 100 | B-1 | 10.0 | C-21 | 5.0 | E-1 | 3.0 | D-1/D-2/D-3 | 2240/960/30 |
| Example 22 | J-22 | A-1 | 100 | B-1 | 10.0 | C-22 | 5.0 | E-1 | 3.0 | D-1/D-2/D-3 | 2240/960/30 |
| Example 23 | J-23 | A-1 | 100 | B-1 | 10.0 | C-23 | 5.0 | E-1 | 3.0 | D-1/D-2/D-3 | 2240/960/30 |
| Example 24 | J-24 | A-2 | 100 | B-1 | 10.0 | C-1 | 5.0 | E-1 | 3.0 | D-1/D-2/D-3 | 2240/960/30 |
| Example 25 | J-25 | A-3 | 100 | B-1 | 10.0 | C-1 | 5.0 | E-1 | 3.0 | D-1/D-2/D-3 | 2240/960/30 |
| Example 26 | J-26 | A-4 | 100 | B-1 | 10.0 | C-1 | 5.0 | E-1 | 3.0 | D-1/D-2/D-3 | 2240/960/30 |
| Example 27 | J-27 | A-5 | 100 | B-1 | 10.0 | C-1 | 5.0 | E-1 | 3.0 | D-1/D-2/D-3 | 2240/960/30 |
| Example 28 | J-28 | A-6 | 100 | B-1 | 10.0 | C-1 | 5.0 | E-1 | 3.0 | D-1/D-2/D-3 | 2240/960/30 |
| Example 29 | J-29 | A-7 | 100 | B-1 | 10.0 | C-1 | 5.0 | E-1 | 3.0 | D-1/D-2/D-3 | 2240/960/30 |
| Example 30 | J-30 | A-8 | 100 | B-1 | 10.0 | C-1 | 5.0 | E-1 | 3.0 | D-1/D-2/D-3 | 2240/960/30 |
| Example 31 | J-31 | A-9 | 100 | B-1 | 10.0 | C-1 | 5.0 | E-1 | 3.0 | D-1/D-2/D-3 | 2240/960/30 |
| Example 32 | J-32 | A-10 | 100 | B-1 | 10.0 | C-1 | 5.0 | E-1 | 3.0 | D-1/D-2/D-3 | 2240/960/30 |
| Example 33 | J-33 | A-11 | 100 | B-1 | 10.0 | C-1 | 5.0 | E-1 | 3.0 | D-1/D-2/D-3 | 2240/960/30 |
| Example 34 | J-34 | A-1 | 100 | B-2 | 10.0 | C-1 | 5.0 | E-1 | 3.0 | D-1/D-2/D-3 | 2240/960/30 |
| Example 35 | J-35 | A-1 | 100 | B-3 | 10.0 | C-1 | 5.0 | E-1 | 3.0 | D-1/D-2/D-3 | 2240/960/30 |
| Example 36 | J-36 | A-1 | 100 | B-4 | 10.0 | C-1 | 5.0 | E-1 | 3.0 | D-1/D-2/D-3 | 2240/960/30 |
| Example 37 | J-37 | A-1 | 100 | B-5 | 10.0 | C-1 | 5.0 | E-1 | 3.0 | D-1/D-2/D-3 | 2240/960/30 |
| Example 38 | J-38 | A-1 | 100 | B-6 | 10.0 | C-1 | 5.0 | E-1 | 3.0 | D-1/D-2/D-3 | 2240/960/30 |
| Example 39 | J-39 | A-1 | 100 | B-7 | 10.0 | C-1 | 5.0 | E-1 | 3.0 | D-1/D-2/D-3 | 2240/960/30 |
| Example 40 | J-40 | A-1 | 100 | B-8 | 10.0 | C-1 | 5.0 | E-1 | 3.0 | D-1/D-2/D-3 | 2240/960/30 |
| Example 41 | J-41 | A-1 | 100 | B-1 | 10.0 | C-1 | 5.0 | E-2 | 3.0 | D-1/D-2/D-3 | 2240/960/30 |
| Example 42 | J-42 | A-1 | 100 | B-1 | 10.0 | C-1 | 5.0 | E-3 | 3.0 | D-1/D-2/D-3 | 2240/960/30 |
| Example 43 | J-43 | A-1 | 100 | B-1 | 10.0 | C-1 | 5.0 | E-4 | 3.0 | D-1/D-2/D-3 | 2240/960/30 |
| Example 44 | J-44 | A-1 | 100 | B-1 | 10.0 | C-1 | 0.5 | E-1 | 3.0 | D-1/D-2/D-3 | 2240/960/30 |
| Example 45 | J-45 | A-1 | 100 | B-1 | 10.0 | C-1 | 2.0 | E-1 | 3.0 | D-1/D-2/D-3 | 2240/960/30 |
| Example 46 | J-46 | A-1 | 100 | B-1 | 10.0 | C-1 | 15.0 | E-1 | 3.0 | D-1/D-2/D-3 | 2240/960/30 |
| Example 47 | J-47 | A-1 | 100 | B-1 | 10.0 | C-1/C-11 | 2.5/2.5 | E-1 | 3.0 | D-1/D-2/D-3 | 2240/960/30 |
| Example 48 | J-48 | A-1 | 100 | B-1 | 10.0 | C-1/C-16 | 2.5/2.5 | E-1 | 3.0 | D-1/D-2/D-3 | 2240/960/30 |
| Example 49 | J-49 | A-1 | 100 | B-1 | 10.0 | C-4/C-23 | 2.5/2.5 | E-1 | 3.0 | D-1/D-2/D-3 | 2240/960/30 |
| Example 50 | J-50 | A-1 | 100 | B-1 | 10.0 | C-1/cc-2 | 2.5/2.5 | E-1 | 3.0 | D-1/D-2/D-3 | 2240/960/30 |
| Example 51 | J-51 | A-1 | 100 | B-1/B-4 | 5.0/5.0 | C-1 | 5.0 | E-1 | 3.0 | D-1/D-2/D-3 | 2240/960/30 |
| Example 52 | J-52 | A-1 | 100 | B-1/B-6 | 5.0/5.0 | C-1 | 5.0 | E-1 | 3.0 | D-1/D-2/D-3 | 2240/960/30 |
| Example 53 | J-53 | A-1 | 100 | B-1/B-8 | 5.0/5.0 | C-1 | 5.0 | E-1 | 3.0 | D-1/D-2/D-3 | 2240/960/30 |
| Comparative | CJ-1 | A-1 | 100 | B-1 | 10.0 | cc-1 | 5.0 | E-1 | 3.0 | D-1/D-2/D-3 | 2240/960/30 |
| Example 1 | | | | | | | | | | | |
| Comparative | CJ-2 | A-1 | 100 | B-1 | 10.0 | cc-2 | 5.0 | E-1 | 3.0 | D-1/D-2/D-3 | 2240/960/30 |
| Example 2 | | | | | | | | | | | |
| Comparative | CJ-3 | A-1 | 100 | B-1 | 10.0 | cc-3 | 5.0 | E-1 | 3.0 | D-1/D-2/D-3 | 2240/960/30 |
| Example 3 | | | | | | | | | | | |
| Comparative | CJ-4 | A-1 | 100 | B-1 | 10.0 | cc-4 | 5.0 | E-1 | 3.0 | D-1/D-2/D-3 | 2240/960/30 |
| Example 4 | | | | | | | | | | | |
| Comparative | CJ-5 | A-1 | 100 | B-1 | 10.0 | cc-5 | 5.0 | E-1 | 3.0 | D-1/D-2/D-3 | 2240/960/30 |
| Example 5 | | | | | | | | | | | |
| Comparative | CJ-6 | A-1 | 100 | B-1 | 10.0 | cc-6 | 5.0 | E-1 | 3.0 | D-1/D-2/D-3 | 2240/960/30 |
| Example 6 | | | | | | | | | | | |
| Comparative | CJ-7 | A-1 | 100 | B-1 | 10.0 | cc-7 | 5.0 | E-1 | 3.0 | D-1/D-2/D-3 | 2240/960/30 |
| Example 7 | | | | | | | | | | | |
| Comparative | CJ-8 | A-1 | 100 | B-1 | 10.0 | cc-8 | 5.0 | E-1 | 3.0 | D-1/D-2/D-3 | 2240/960/30 |
| Example 8 | | | | | | | | | | | |
| Comparative | CJ-9 | A-1 | 100 | B-1 | 10.0 | cc-9 | 5.0 | E-1 | 3.0 | D-1/D-2/D-3 | 2240/960/30 |
| Example 9 | | | | | | | | | | | |
| Comparative | CJ-10 | A-1 | 100 | B-1 | 10.0 | cc-10 | 5.0 | E-1 | 3.0 | D-1/D-2/D-3 | 2240/960/30 |
| Example 10 | | | | | | | | | | | |

### <Formation of Resist Pattern Using Negative Radiation-Sensitive Resin Composition for ArF Exposure>

Onto the surface of a 12-inch silicon wafer, an underlayer antireflection film forming composition (“ARC66” manufactured by Brewer Science Incorporated.) was applied with use of a spin coater (“CLEAN TRACK ACT12” manufactured by Tokyo Electron Limited.). The wafer was then heated at 205° C. for 60 seconds to form an underlayer antireflection film having an average thickness of 100 nm.

The negative radiation-sensitive resin composition for ArF exposure prepared above was applied onto the underlayer antireflection film with use of the spin coater, followed by performing PB (pre-baking) at 100° C. for 60 seconds. Thereafter, cooling was performed at 23° C. for 30 seconds to form a resist film having an average thickness of 90 nm. Next, this resist film was exposed through a mask pattern having a 40 nm hole and a 105 nm pitch using an ArF excimer laser immersion exposure apparatus (“TWINSCAN XT-1900i” manufactured by ASML) with NA of 1.35 under an optical condition of Dipole (σ=0.9/0.7).

After the exposure, PEB (post exposure baking) was performed at 100° C. for 60 seconds. Thereafter, the resist film was developed with an organic solvent using n-butyl acetate as an organic solvent developer, and dried to form a negative resist pattern (40 nm hole, 105 nm pitch).

### <Evaluation>

The resist pattern formed using the negative radiation-sensitive resin composition for ArF exposure was evaluated on sensitivity, LWR performance, and pattern rectangularity in accordance with the following methods. The results are shown in the following Table 5. It is to be noted that a scanning electron microscope (“CG-5000” manufactured by Hitachi High-Tech Corporation) was used for measurement of the resist pattern.

### [Sensitivity]

An exposure dose at which a 40 nm hole pattern was formed in formation of a resist pattern using the negative radiation-sensitive resin composition for ArF exposure was defined as an optimum exposure dose, and this optimum exposure dose was defined as sensitivity (mJ/cm²). The sensitivity was evaluated to be “good” in a case of being 30 mJ/cm² or less, and “poor” in a case of exceeding 30 mJ/cm².

### [Cdu Performance]

A resist pattern with 40 nm holes and 105 nm pitches was measured using the scanning electron microscope, and measurement was performed at any 1,800 points in total from above the pattern. The dimensional variation (30) was determined and taken as the CDU performance (nm). A smaller value of CDU indicates smaller variation in the hole diameter in the long period and better performance. When the value was 2.5 nm or less, the CDU performance was evaluated as “good”, and when the value exceeded 2.5 nm, the CDU performance was evaluated as “poor”.

### [Depth of Focus]

In a resist pattern to be resolved with the optimum exposure amount determined in the above-described evaluation of sensitivity, the dimension when the focus was changed in the depth direction was observed, and the margin in the depth direction in which the pattern dimension fell within 90% to 110% of the reference without any bridge or residue was measured. The measured value was taken as the depth of focus (nm). The larger the measured value, the better the depth of focus. When the measured value is 70 nm or more, the depth of focus can be evaluated as “good”, and when the measured value is less than 70 nm, the depth of focus can be evaluated as “poor”.

### [Pattern Rectangularity]

The 40 nm hole-and-space resist pattern formed by irradiation with the optimum exposure amount obtained in the evaluation of the sensitivity was observed using the scanning electron microscope, and the cross-sectional shape of the hole pattern was evaluated. The rectangularity of the resist pattern was evaluated as “A” (extremely good) when the ratio of the length of the upper side to the length of the upper side in the cross-sectional shape was 1 or more and 1.05 or less, “B” (good) when the ratio was more than 1.05 and 1.10 or less, and “C” (poor) when the ratio was more than 1.10.

TABLE 5

| | Radiation- | | | | |
|---|---|---|---|---|---|
| | sensitive | | | Depth | |
| | resin | Sensitivity | CDU | of focus | Pattern |
| | composition | (mJ/cm²) | (nm) | (nm) | rectangularity |
| Example 1 | J-1 | 25 | 2.0 | 90 | A |
| Example 2 | J-2 | 24 | 2.2 | 90 | A |
| Example 3 | J-3 | 23 | 2.2 | 80 | A |
| Example 4 | J-4 | 23 | 1.9 | 100 | A |
| Example 5 | J-5 | 26 | 2.1 | 110 | A |
| Example 6 | J-6 | 25 | 2.0 | 90 | A |
| Example 7 | J-7 | 27 | 2.3 | 80 | A |
| Example 8 | J-8 | 29 | 2.4 | 110 | A |
| Example 9 | J-9 | 28 | 2.4 | 100 | A |
| Example 10 | J-10 | 28 | 1.9 | 100 | A |
| Example 11 | J-11 | 23 | 1.8 | 100 | A |
| Example 12 | J-12 | 28 | 2.2 | 90 | A |
| Example 13 | J-13 | 24 | 2.3 | 90 | A |
| Example 14 | J-14 | 26 | 2.0 | 80 | A |
| Example 15 | J-15 | 26 | 2.1 | 110 | A |
| Example 16 | J-16 | 23 | 2.3 | 100 | A |
| Example 17 | J-17 | 22 | 2.2 | 80 | A |
| Example 18 | J-18 | 26 | 2.0 | 100 | A |
| Example 19 | J-19 | 28 | 2.1 | 90 | A |
| Example 20 | J-20 | 27 | 1.9 | 90 | A |
| Example 21 | J-21 | 27 | 2.3 | 80 | A |
| Example 22 | J-22 | 26 | 2.1 | 110 | A |
| Example 23 | J-23 | 25 | 2.1 | 100 | A |
| Example 24 | J-24 | 26 | 1.9 | 90 | A |
| Example 25 | J-25 | 25 | 2.0 | 80 | A |
| Example 26 | J-26 | 24 | 2.1 | 90 | A |
| Example 27 | J-27 | 25 | 2.1 | 90 | A |
| Example 28 | J-28 | 26 | 2.2 | 100 | A |
| Example 29 | J-29 | 27 | 1.9 | 80 | A |
| Example 30 | J-30 | 22 | 2.3 | 90 | A |
| Example 31 | J-31 | 26 | 2.0 | 80 | A |
| Example 32 | J-32 | 25 | 2.1 | 90 | A |
| Example 33 | J-33 | 24 | 2.4 | 90 | A |
| Example 34 | J-34 | 23 | 2.3 | 90 | A |
| Example 35 | J-35 | 23 | 2.2 | 90 | A |
| Example 36 | J-36 | 26 | 2.0 | 100 | A |
| Example 37 | J-37 | 27 | 1.9 | 90 | A |
| Example 38 | J-38 | 26 | 1.9 | 80 | A |
| Example 39 | J-39 | 23 | 2.2 | 90 | A |
| Example 40 | J-40 | 28 | 2.3 | 90 | A |
| Example 41 | J-41 | 25 | 2.0 | 90 | A |
| Example 42 | J-42 | 25 | 2.1 | 90 | A |
| Example 43 | J-43 | 26 | 2.0 | 90 | A |
| Example 44 | J-44 | 20 | 2.4 | 90 | A |
| Example 45 | J-45 | 23 | 2.2 | 90 | A |
| Example 46 | J-46 | 28 | 1.9 | 90 | A |
| Example 47 | J-47 | 25 | 2.0 | 80 | A |
| Example 48 | J-48 | 23 | 2.2 | 90 | A |
| Example 49 | J-49 | 23 | 2.0 | 100 | A |
| Example 50 | J-50 | 27 | 2.3 | 80 | A |
| Example 51 | J-51 | 26 | 2.4 | 80 | A |
| Example 52 | J-52 | 26 | 2.1 | 100 | A |
| Example 53 | J-53 | 28 | 1.8 | 80 | A |
| Comparative | CJ-1 | 33 | 2.7 | 50 | B |
| Example 1 | | | | | |
| Comparative | CJ-2 | 35 | 2.7 | 60 | B |
| Example 2 | | | | | |
| Comparative | CJ-3 | 32 | 2.8 | 50 | C |
| Example 3 | | | | | |
| Comparative | CJ-4 | 35 | 3.0 | 50 | C |
| Example 4 | | | | | |
| Comparative | CJ-5 | 33 | 3.1 | 60 | B |
| Example 5 | | | | | |
| Comparative | CJ-6 | 32 | 2.8 | 50 | C |
| Example 6 | | | | | |
| Comparative | CJ-7 | 32 | 2.9 | 50 | C |
| Example 7 | | | | | |
| Comparative | CJ-8 | 33 | 3.0 | 60 | C |
| Example 8 | | | | | |
| Comparative | CJ-9 | 36 | 3.1 | 40 | B |
| Example 9 | | | | | |
| Comparative | CJ-10 | 40 | 3.3 | 40 | C |
| Example 10 | | | | | |

As is apparent from the results in Table 5, the radiation-sensitive resin compositions of Examples were good in sensitivity, CDU performance, depth of focus, and pattern rectangularity when used for ArF exposure, whereas the radiation-sensitive resin compositions of Comparative Examples were poorer in the characteristics than those of Examples. Therefore, when the radiation-sensitive resin compositions of Examples are used for ArF exposure, resist patterns high in sensitivity, good in CDU performance and depth of focus, and superior in rectangularity can be formed.

### [Preparation of Radiation-Sensitive Resin Composition for Extreme Ultraviolet Ray (EUV) Exposure]

### Example 54

100 parts by mass of (A-12) as the resin [A], 15.0 parts by mass of (B-7) as the radiation-sensitive acid generator [B], 8.0 parts by mass of (C-1) as the acid diffusion controlling agent [C], 3.0 parts by mass (solid content) of (E-5) as the high fluorine-content resin [E], and 6,110 parts by mass of a mixed solvent of (D-1)/(D-4) as the solvent [D] were mixed, and the mixture was filtered through a membrane filter having a pore size of 0.2 μm to prepare a radiation-sensitive resin composition (J-54).

### Examples 55 to 66 and Comparative Examples 11 to 14

Radiation-sensitive resin compositions (J-55) to (J-66) and (CJ-11) to (CJ-14) were prepared in the same manner as in Example 54 except that the components of the types and contents shown in the following Table 6 were used.

TABLE 6

| | | | | | | Acid diffusion | | | | | |
|---|---|---|---|---|---|---|---|---|---|---|---|
| | Radiation- | Polymer [A] | | Acid generator [B] | | controlling agent [C] | | Polymer [E] | | Organic solvent [D] | |
| | sensitive | | Content | | Content | | Content | | Content | | Content |
| | resin | | (parts | | (parts | | (parts | | (parts | | (parts |
| | composition | Type | by mass) | Type | by mass) | Type | by mass) | Type | by mass) | Type | by mass) |
| Example 54 | J-54 | A-12 | 100 | B-7 | 15.0 | C-1 | 8.0 | E-5 | 3.0 | D-1/D-4 | 4280/1830 |
| Example 55 | J-55 | A-12 | 100 | B-7 | 15.0 | C-4 | 8.0 | E-5 | 3.0 | D-1/D-4 | 4280/1830 |
| Example 56 | J-56 | A-12 | 100 | B-7 | 15.0 | C-5 | 8.0 | E-5 | 3.0 | D-1/D-4 | 4280/1830 |
| Example 57 | J-57 | A-12 | 100 | B-7 | 15.0 | C-11 | 8.0 | E-5 | 3.0 | D-1/D-4 | 4280/1830 |
| Example 58 | J-58 | A-12 | 100 | B-7 | 15.0 | C-15 | 8.0 | E-5 | 3.0 | D-1/D-4 | 4280/1830 |
| Example 59 | J-59 | A-12 | 100 | B-7 | 15.0 | C-18 | 8.0 | E-5 | 3.0 | D-1/D-4 | 4280/1830 |
| Example 60 | J-60 | A-12 | 100 | B-7 | 15.0 | C-23 | 8.0 | E-5 | 3.0 | D-1/D-4 | 4280/1830 |
| Example 61 | J-61 | A-13 | 100 | B-7 | 15.0 | C-1 | 8.0 | E-5 | 3.0 | D-1/D-4 | 4280/1830 |
| Example 62 | J-62 | A-14 | 100 | B-7 | 15.0 | C-1 | 8.0 | E-5 | 3.0 | D-1/D-4 | 4280/1830 |
| Example 63 | J-63 | A-15 | 100 | B-7 | 15.0 | C-1 | 8.0 | E-5 | 3.0 | D-1/D-4 | 4280/1830 |
| Example 64 | J-64 | A-12 | 100 | B-1 | 15.0 | C-1 | 8.0 | E-5 | 3.0 | D-1/D-4 | 4280/1830 |
| Example 65 | J-65 | A-12 | 100 | B-3 | 15.0 | C-1 | 8.0 | E-5 | 3.0 | D-1/D-4 | 4280/1830 |
| Example 66 | J-66 | A-12 | 100 | B-5/B-8 | 7.5/7.5 | C-1 | 8.0 | E-5 | 3.0 | D-1/D-4 | 4280/1830 |
| Comparative | CJ-11 | A-12 | 100 | B-7 | 15.0 | cc-1 | 8.0 | E-5 | 3.0 | D-1/D-4 | 4280/1830 |
| Example 11 | | | | | | | | | | | |
| Comparative | CJ-12 | A-12 | 100 | B-7 | 15.0 | cc-2 | 8.0 | E-5 | 3.0 | D-1/D-4 | 4280/1830 |
| Example 12 | | | | | | | | | | | |
| Comparative | CJ-13 | A-12 | 100 | B-7 | 15.0 | cc-4 | 8.0 | E-5 | 3.0 | D-1/D-4 | 4280/1830 |
| Example 13 | | | | | | | | | | | |
| Comparative | CJ-14 | A-12 | 100 | B-7 | 15.0 | cc-9 | 8.0 | E-5 | 3.0 | D-1/D-4 | 4280/1830 |
| Example 14 | | | | | | | | | | | |

### <Formation of Resist Pattern Using Radiation-Sensitive Resin Composition for EUV Exposure>

Onto the surface of a 12-inch silicon wafer, an underlayer antireflection film forming composition (“ARC66” manufactured by Brewer Science Incorporated.) was applied with use of a spin coater (“CLEAN TRACK ACT12” manufactured by Tokyo Electron Limited.). The wafer was then heated at 205° C. for 60 seconds to form an underlayer antireflection film having an average thickness of 105 nm.

The radiation-sensitive resin composition for EUV exposure prepared above was applied onto the underlayer antireflection film with use of the spin coater, followed by performing PB at 130° C. for 60 seconds. Thereafter, cooling was performed at 23° C. for 30 seconds to form a resist film having an average thickness of 55 nm. Next, the resist film was exposed by an EUV exposure apparatus (“NXE3300”, manufactured by ASML) with NA of 0.33 under a lighting condition of Conventional s=0.89 and with a mask of imecDEFECT32FFR02.

After exposing, PEB was performed at 120° C. for 60 seconds. Thereafter, the resist film was developed with an alkali with use of a 2.38% by mass aqueous TMAH solution as an alkaline developer, followed by washing with water and further drying to form a positive resist pattern (32 nm line-and-space pattern).

### <Evaluation>

The resist patterns formed using the radiation-sensitive resin compositions for EUV exposure were evaluated on sensitivity and LWR performance according to the following methods. The results are shown in the following Table 7. It is to be noted that a scanning electron microscope (“CG-5000” manufactured by Hitachi High-Tech Corporation) was used for measurement of the resist pattern.

### [Sensitivity]

An exposure dose at which a 32 nm line-and-space pattern was formed in the aforementioned resist pattern formation using the radiation-sensitive resin composition for EUV exposure was defined as an optimum exposure dose, and this optimum exposure dose was defined as sensitivity (mJ/cm²). The sensitivity was evaluated to be “good” in a case of being 25 mJ/cm² or less, and “poor” in a case of exceeding 25 mJ/cm².

### [LWR Performance]

A resist pattern was formed by adjusting a mask size so as to form a 32 nm line-and-space pattern by irradiation with the optimum exposure dose obtained in the evaluation of the sensitivity. The formed resist pattern was observed from above the pattern with use of the scanning electron microscope. The variation in line width was measured at 500 points in total, the value of 3σ was obtained from the distribution of the measured values, and the value of 3σ was defined as LWR (nm). The smaller the value of the LWR is, the smaller the wobble of the line is, which is better. The LWR performance was evaluated as “good” when the LWR was 2.5 nm or less, and was evaluated as “poor” when the LWR exceeded 2.5 nm.

TABLE 7

| | Radiation-sensitive | Sensitivity | LWR |
|---|---|---|---|
| | resin composition | (mJ/cm²) | (nm) |
| Example 54 | J-54 | 21 | 2.1 |
| Example 55 | J-55 | 23 | 1.8 |
| Example 56 | J-56 | 19 | 2.0 |
| Example 57 | J-57 | 22 | 2.0 |
| Example 58 | J-58 | 22 | 2.1 |
| Example 59 | J-59 | 23 | 2.2 |
| Example 60 | J-60 | 19 | 2.0 |
| Example 61 | J-61 | 20 | 2.0 |
| Example 62 | J-62 | 20 | 2.1 |
| Example 63 | J-63 | 21 | 2.1 |
| Example 64 | J-64 | 22 | 2.2 |
| Example 65 | J-65 | 23 | 2.0 |
| Example 66 | J-66 | 20 | 2.1 |
| Comparative | CJ-11 | 30 | 2.8 |
| Example 11 | | | |
| Comparative | CJ-12 | 31 | 3.0 |
| Example 12 | | | |
| Comparative | CJ-13 | 28 | 3.1 |
| Example 13 | | | |
| Comparative | CJ-14 | 29 | 3.0 |
| Example 14 | | | |

As is apparent from the results in Table 7, the radiation-sensitive resin compositions of Examples were good in sensitivity and LWR performance when used for EUV exposure, whereas the radiation-sensitive resin compositions of Comparative Examples were poorer in the characteristics than those of Examples.

### [Preparation of Positive Radiation-Sensitive Resin Composition for ArF Exposure]

### Example 67

100 parts by mass of (A-6) as the resin [A], 10.0 parts by mass of (B-2) as the radiation-sensitive acid generator [B], 8.0 parts by mass of (C-1) as the acid diffusion controlling agent [C], 5.0 parts by mass (solid content) of (E-2) as the high fluorine-content resin [E], and 3,230 parts by mass of a mixed solvent of (D-1)/(D-2)/(D-3) as the solvent [D] were mixed, and the mixture was filtered through a membrane filter having a pore size of 0.2 μm to prepare a radiation-sensitive resin composition (J-67).

### Examples 68 to 79 and Comparative Examples 15 to 18

Radiation-sensitive resin compositions (J-68) to (J-79) and (CJ-15) to (CJ-18) were prepared in the same manner as in Example 66 except that the components of the types and contents shown in the following Table 8 were used.

TABLE 8

| | | | | | | Acid diffusion | | | | | |
|---|---|---|---|---|---|---|---|---|---|---|---|
| | Radiation- | Polymer [A] | | Acid generator [B] | | controlling agent [C] | | Polymer [E] | | Organic solvent [D] | |
| | sensitive | | Content | | Content | | Content | | Content | | Content |
| | resin | | (parts | | (parts | | (parts | | (parts | | (parts |
| | composition | Type | by mass) | Type | by mass) | Type | by mass) | Type | by mass) | Type | by mass) |
| Example 67 | J-67 | A-6 | 100 | B-2 | 10.0 | C-1 | 8.0 | E-2 | 5.0 | D-1/D-2/D-3 | 2240/960/30 |
| Example 68 | J-68 | A-6 | 100 | B-2 | 10.0 | C-2 | 8.0 | E-2 | 5.0 | D-1/D-2/D-3 | 2240/960/30 |
| Example 69 | J-69 | A-6 | 100 | B-2 | 10.0 | C-4 | 8.0 | E-2 | 5.0 | D-1/D-2/D-3 | 2240/960/30 |
| Example 70 | J-70 | A-6 | 100 | B-2 | 10.0 | C-11 | 8.0 | E-2 | 5.0 | D-1/D-2/D-3 | 2240/960/30 |
| Example 71 | J-71 | A-6 | 100 | B-2 | 10.0 | C-17 | 8.0 | E-2 | 5.0 | D-1/D-2/D-3 | 2240/960/30 |
| Example 72 | J-72 | A-6 | 100 | B-2 | 10.0 | C-20 | 8.0 | E-2 | 5.0 | D-1/D-2/D-3 | 2240/960/30 |
| Example 73 | J-73 | A-6 | 100 | B-2 | 10.0 | C-23 | 8.0 | E-2 | 5.0 | D-1/D-2/D-3 | 2240/960/30 |
| Example 74 | J-74 | A-3 | 100 | B-2 | 10.0 | C-1 | 8.0 | E-2 | 5.0 | D-1/D-2/D-3 | 2240/960/30 |
| Example 75 | J-75 | A-4 | 100 | B-2 | 10.0 | C-1 | 8.0 | E-2 | 5.0 | D-1/D-2/D-3 | 2240/960/30 |
| Example 76 | J-76 | A-7 | 100 | B-2 | 10.0 | C-1 | 8.0 | E-2 | 5.0 | D-1/D-2/D-3 | 2240/960/30 |
| Example 77 | J-77 | A-6 | 100 | B-6 | 10.0 | C-1 | 8.0 | E-2 | 5.0 | D-1/D-2/D-3 | 2240/960/30 |
| Example 78 | J-78 | A-6 | 100 | B-7 | 10.0 | C-1 | 8.0 | E-2 | 5.0 | D-1/D-2/D-3 | 2240/960/30 |
| Example 79 | J-79 | A-6 | 100 | B-5/B-8 | 5.0/5.0 | C-1 | 8.0 | E-2 | 5.0 | D-1/D-2/D-3 | 2240/960/30 |
| Comparative | CJ-15 | A-6 | 100 | B-2 | 10.0 | cc-1 | 8.0 | E-2 | 5.0 | D-1/D-2/D-3 | 2240/960/30 |
| Example 15 | | | | | | | | | | | |
| Comparative | CJ-16 | A-6 | 100 | B-2 | 10.0 | cc-2 | 8.0 | E-2 | 5.0 | D-1/D-2/D-3 | 2240/960/30 |
| Example 16 | | | | | | | | | | | |
| Comparative | CJ-17 | A-6 | 100 | B-2 | 10.0 | cc-8 | 8.0 | E-2 | 5.0 | D-1/D-2/D-3 | 2240/960/30 |
| Example 17 | | | | | | | | | | | |
| Comparative | CJ-18 | A-6 | 100 | B-2 | 10.0 | cc-9 | 8.0 | E-2 | 5.0 | D-1/D-2/D-3 | 2240/960/30 |
| Example 18 | | | | | | | | | | | |

Onto the surface of a 12-inch silicon wafer, an underlayer antireflection film forming composition (“ARC66” manufactured by Brewer Science Incorporated.) was applied with use of a spin coater (“CLEAN TRACK ACT12” manufactured by Tokyo Electron Limited.). The wafer was then heated at 205° C. for 60 seconds to form an underlayer antireflection film having an average thickness of 100 nm.

The positive radiation-sensitive resin composition for ArF exposure prepared above was applied onto the underlayer antireflection film with use of the spin coater, followed by performing PB (pre-baking) at 100° C. for 60 seconds. Thereafter, cooling was performed at 23° C. for 30 seconds to form a resist film having an average thickness of 90 nm. Next, this resist film was exposed through a 50 nm line-and-space mask pattern using an ArF excimer laser immersion exposure apparatus (“TWINSCAN XT-1900i” manufactured by ASML) with NA of 1.35 under an optical condition of Annular (σ=0.8/0.6).

After the exposure, PEB (post exposure baking) was performed at 100° C. for 60 seconds. Thereafter, the resist film was developed with an alkali with use of a 2.38% by mass aqueous TMAH solution as an alkaline developer, followed by washing with water and further drying to form a positive resist pattern (50 nm line-and-space pattern).

### <Evaluation>

The resist patterns formed using the radiation-sensitive resin compositions for ArF exposure were evaluated on sensitivity and LWR performance according to the following methods. The results are shown in the following Table 9. It is to be noted that a scanning electron microscope (“CG-5000” manufactured by Hitachi High-Tech Corporation) was used for measurement of the resist pattern.

### [Sensitivity]

An exposure dose at which a 50 nm line-and-space pattern was formed in the aforementioned resist pattern formation using each of the radiation-sensitive resin compositions for ArF exposure was defined as an optimum exposure dose, and this optimum exposure dose was defined as sensitivity (mJ/cm²). The sensitivity was evaluated to be “good” in a case of being 30 mJ/cm² or less, and “poor” in a case of exceeding 30 mJ/cm².

### [Lwr Performance]

A resist pattern was formed by adjusting a mask size so as to form a 50 nm line-and-space pattern by irradiation with the optimum exposure dose obtained in the evaluation of the sensitivity. The formed resist pattern was observed from above the pattern with use of the scanning electron microscope. The variation in line width was measured at 500 points in total, the value of 3σ was obtained from the distribution of the measured values, and the value of 3σ was defined as LWR (nm). The smaller the value of the LWR is, the smaller the wobble of the line is, which is better. The LWR performance was evaluated as “good” when the LWR was 2.0 nm or less, and was evaluated as “poor” when the LWR exceeded 2.0 nm.

TABLE 9

| | Radiation-sensitive | Sensitivity | LWR |
|---|---|---|---|
| | resin composition | (mJ/cm²) | (nm) |
| Example 67 | J-67 | 27 | 1.8 |
| Example 68 | J-68 | 26 | 1.8 |
| Example 69 | J-69 | 27 | 1.7 |
| Example 70 | J-70 | 25 | 1.8 |
| Example 71 | J-71 | 28 | 1.7 |
| Example 72 | J-72 | 27 | 1.8 |
| Example 73 | J-73 | 26 | 1.9 |
| Example 74 | J-74 | 29 | 1.6 |
| Example 75 | J-75 | 26 | 1.7 |
| Example 76 | J-76 | 27 | 1.8 |
| Example 77 | J-77 | 28 | 1.8 |
| Example 78 | J-78 | 26 | 1.8 |
| Example 79 | J-79 | 27 | 1.7 |
| Comparative | CJ-15 | 32 | 2.9 |
| Example 15 | | | |
| Comparative | CJ-16 | 35 | 2.5 |
| Example 16 | | | |
| Comparative | CJ-17 | 32 | 3.0 |
| Example 17 | | | |
| Comparative | CJ-18 | 34 | 2.8 |
| Example 18 | | | |

As is apparent from the results in Table 9, the radiation-sensitive resin compositions of Examples were good in sensitivity and LWR performance when used for ArF exposure, whereas the radiation-sensitive resin compositions of Comparative Examples were poorer in the characteristics than those of Examples.

[Preparation of Negative Radiation-Sensitive Resin Composition for EUV Exposure, and Formation and Evaluation of Resist Pattern Using this Composition]

### Example 80

100 parts by mass of (A-13) as the resin [A], 18.0 parts by mass of (B-1) as the radiation-sensitive acid generator [B], 10.0 parts by mass of (C-11) as the acid diffusion controlling agent [C], 1.0 parts by mass (solid content) of (E-5) as the high fluorine-content resin [E], and 6,110 parts by mass of a mixed solvent of (D-1)/(D-4) as the solvent [D] were mixed, and the mixture was filtered through a membrane filter having a pore size of 0.2 μm to prepare a radiation-sensitive resin composition (J-80).

Onto the surface of a 12-inch silicon wafer, an underlayer antireflection film forming composition (“ARC66” manufactured by Brewer Science Incorporated.) was applied with use of a spin coater (“CLEAN TRACK ACT12” manufactured by Tokyo Electron Limited.). The wafer was then heated at 205° C. for 60 seconds to form an underlayer antireflection film having an average thickness of 105 nm.

The radiation-sensitive resin composition for EUV exposure (J-80) prepared above was applied onto the underlayer antireflection film with use of the spin coater, followed by performing PB at 130° C. for 60 seconds. Thereafter, cooling was performed at 23° C. for 30 seconds to form a resist film having an average thickness of 55 nm. Next, the resist film was exposed by an EUV exposure apparatus (“NXE3300”, manufactured by ASML) with NA of 0.33 under a lighting condition of Conventional s=0.89 and with a mask of imecDEFECT32FFR02.

After exposing, PEB was performed at 120° C. for 60 seconds. Thereafter, the resist film was developed with an organic solvent using n-butyl acetate as an organic solvent developer, and dried to form a negative resist pattern (40 nm hole, 105 nm pitch).

The resist pattern using the negative radiation-sensitive resin composition for EUV exposure was evaluated in the same manner as in the evaluation of the resist pattern using the negative radiation-sensitive resin composition for ArF exposure. As a result, the radiation-sensitive resin composition of Example 80 had good sensitivity and CDU performance even when a negative resist pattern was formed by EUV exposure.

According to the radiation-sensitive resin composition, the resist pattern formation method described above, and so on, a resist pattern having good sensitivity to exposure light and superior CDU performance can be formed. Therefore, these can be suitably used for a machining process and the like of a semiconductor device in which micronization is expected to further progress in the future.

Obviously, numerous modifications and variations of the present invention(s) are possible in light of the above teachings. It is therefore to be understood that within the scope of the appended claims, the invention(s) may be practiced otherwise than as specifically described herein.

1. A radiation-sensitive resin composition comprising:
a compound A represented by formula (I);
a resin B comprising a structural unit having an acid-dissociable group;
a radiation-sensitive acid generator other than the compound A; and
a solvent: 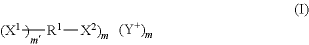
wherein
R¹ is an (m+m′)-valent organic group and comprises a cyclopropane ring skeleton, a cyclobutane ring skeleton, or both,
X¹ is a group represented by formula (1-1) or a group represented by formula (1-2),
X² is a group represented by formula (2-1) or a group represented by formula (2-2),
Y⁺ is a monovalent onium cation,
m is an integer of 1 to 2, and
m′ is an integer of 0 to 1, 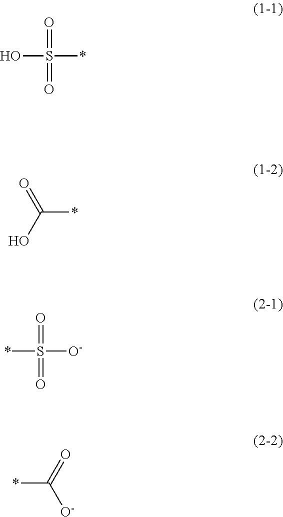
wherein
represents a bond to another group.

2. The radiation-sensitive resin composition according to claim 1, wherein the compound A is a compound represented by formula (1) or formula (2), 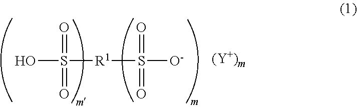
wherein R¹, Y⁺, m, and m′ are each as defined in the formula (I), 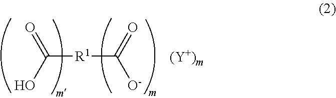
wherein R¹, Y⁺, m, and m′ are each as defined in the formula (I).

3. The radiation-sensitive resin composition according to claim 1, wherein the compound A is represented by formula (3), 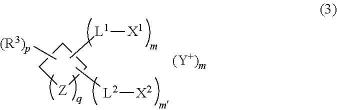
wherein
R³ is a monovalent organic group, a fluorine atom, or a hydroxy group,
L¹ and L² are each independently a single bond or a divalent organic group,
X¹, X², Y⁺, m, and m′ are each as defined in the formula (I),
Z is a divalent group represented by —C(R⁴)₂— or —CO—,
each R⁴ is independently at each occurrence a hydrogen atom, a monovalent organic group, a fluorine atom, or a hydroxy group,
q is an integer of 0 to 1, and
p is an integer of 0 to (6−m−m′).

4. The radiation-sensitive resin composition according to claim 3, wherein the compound A is represented by formula (4-1), formula (4-2), or formula (4-3), 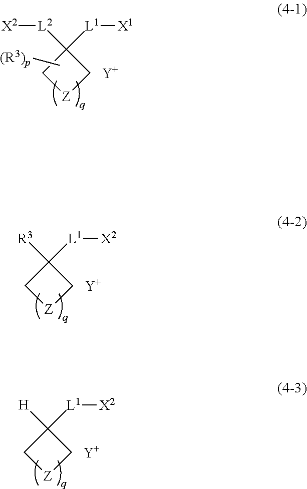
wherein L¹, L², X¹, X², R³, Y⁺, Z, m, m′, p, and q are each as defined in the formula (3).

5. The radiation-sensitive resin composition according to claim 1, wherein Y⁺ is a monovalent radiolytic onium cation.

6. The radiation-sensitive resin composition according to claim 1, wherein the radiation-sensitive acid generator comprises a compound represented by formula (10),
R^{b1}—R^{b2}-SO₃⁻M⁺ (10)
wherein
R^{b1} is a monovalent group comprising an alicyclic structure or a monovalent group comprising an aliphatic heterocyclic structure,
R^{b2} is a fluorinated alkanediyl group having 1 to 10 carbon atoms, and
M⁺ is a monovalent radiolytic onium cation.

7. The radiation-sensitive resin composition according to claim 6, wherein the radiation-sensitive onium cation in the formula (10) is a sulfonium cation or an iodonium cation.

8. The radiation-sensitive resin composition according to claim 1, wherein the structural unit having an acid-dissociable group in the resin B is represented formula (6), 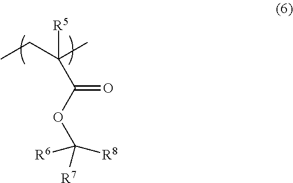
wherein
R⁵ is a hydrogen atom, a fluorine atom, a methyl group, or a trifluoromethyl group,
R⁶ is a monovalent hydrocarbon group having 1 to 20 carbon atoms, and
R⁷ and R⁸ each independently represent a monovalent chain hydrocarbon group having 1 to 10 carbon atoms or a monovalent alicyclic hydrocarbon group having 3 to 20 carbon atoms, or R⁷ and R⁸ taken together represent a divalent alicyclic group having 3 to 20 carbon atoms together with the carbon atoms to which R⁷ and R⁸ are bonded.

9. The radiation-sensitive resin composition according to claim 1, wherein the resin B further comprises a structural unit comprising at least one structure selected from the group consisting of a lactone structure, a cyclic carbonate structure, and a sultone structure.

10. A pattern formation method, comprising:
applying the radiation-sensitive resin composition according to claim 1 directly or indirectly to a substrate to form a resist film;
exposing the resist film to light; and
developing the exposed resist film to form a patterned resist film.

11. The pattern formation method according to claim 10, wherein exposing comprises exposing the resist film to ArF excimer laser light, an extreme ultraviolet ray (EUV), an X-ray, or an electron beam (EB).

12. The pattern formation method according to claim 10, wherein developing comprises developing the exposed resist film with an organic solvent such that a negative-tone pattern is formed.

13. The pattern formation method according to claim 10, wherein developing comprises developing the exposed resist film with an alkaline developer such that a positive-tone pattern is formed.

14. A method for manufacturing a substrate, the method comprising:
forming a pattern on a substrate using the patterned resist film formed by the pattern formation method according to claim 10 as a mask.

15. A compound represented by formula (I): 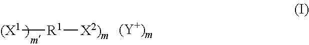
wherein
R¹ is an (m+m′)-valent organic group and comprises a cyclopropane ring skeleton, a cyclobutane ring skeleton, or both,
X¹ is a group represented by formula (1-1) or a group represented by formula (1-2),
X² is a group represented by formula (2-1) or a group represented by formula (2-2),
Y⁺ is a monovalent onium cation,
m is an integer of 1 to 2, and
m′ is an integer of 0 to 1, 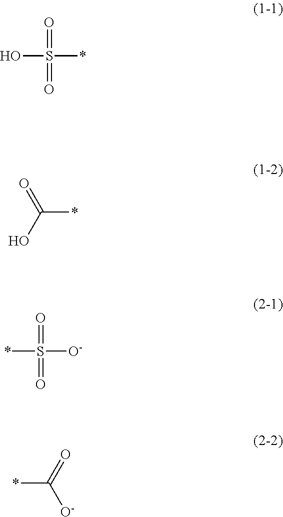
wherein * represents a bond to another group.

16. The compound according to claim 15, wherein the compound is represented by formula (1) or formula (2), 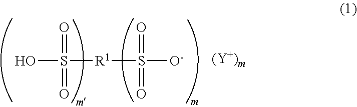
wherein R¹, Y⁺, m, and m′ are each as defined in the formula (I), 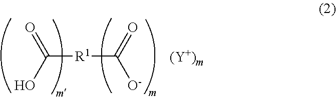
wherein R¹, Y⁺, m, and m′ are each as defined in the formula (I).

17. The compound according to claim 15, wherein the compound is represented by formula (3), 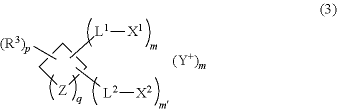
wherein
R³ is a monovalent organic group, a fluorine atom, or a hydroxy group,
L¹ and L² are each independently a single bond or a divalent organic group,
X¹, X², Y⁺, m, and m′ are each as defined in the formula (I),
Z is a divalent group represented by —C(R⁴)₂— or —CO—,
each R⁴ is independently at each occurrence a hydrogen atom, a monovalent organic group, a fluorine atom, or a hydroxy group,
q is an integer of 0 to 1, and
p is an integer of 0 to (6−m−m′).

18. The compound according to claim 17, wherein the compound is represented by formula (4-1), formula (4-2), or formula (4-3), 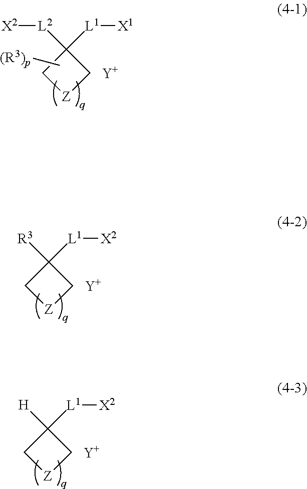
wherein
L¹, L², X¹, X², R³, Y⁺, Z, m, m′, p, and q are each as defined in the formula (3).
